(19) United States
(12) Patent Application Publication
(10) Pub. No.: US 2025/0090475 A1
(43) Pub. Date: Mar. 20, 2025

(54) DRUG-LOADED AMINO ACID BASED POLY (ESTERUREA)FILMS FOR CONTROLLED LOCAL RELEASE OF NON-OPIOID ANALGESIC COMPOUNDS

(71) Applicants: Matthew BECKER, ChapelHill, NC (US); Seth P. FORSTER, Fort Washington, PA (US); Natasha BRIGHAM, Durham, NC (US); Tiffany GUSTAFSON, Fort Worth, TX (US); Andre HERMANS, Metuchen, NJ (US); Rebecca NOFSINGER, Lansdale, PA (US); MERK SHARP & DOHME CORP., Rahway, NJ (US)

(72) Inventors: Matthew BECKER, Chapel Hill, NC (US); Seth P. FORSTER, Forth Washington, PA (US); Natasha BRIGHAM, Durham, NC (US); Tiffany GUSTAFSON, Fort Worth, TX (US); Andre HERMANS, Metuchen, NJ (US); Rebecca NOFSINGER, Lansdale, PA (US)

(73) Assignees: The University of Akron, Akron, OH (US); MERK SHARP & DOHME CORP., Rahway, NJ (US)

(21) Appl. No.: 17/797,567

(22) PCT Filed: Feb. 4, 2021
(86) PCT No.: PCT/US21/16542

§ 371 Date: Aug. 4, 2022

Related U.S. Application Data
(60) Provisional application No. 62/969,919, filed on Feb. 4, 2020.

(51) Int. Cl. A61K 9/70; A61K 47/34

(52) U.S. Cl. CPC A61K 9/7007; A61K 47/34

(57) ABSTRACT

In various embodiments, the present invention provides a drug-loaded amino acid-based poly(ester urea) film for controlled local release of non-opioid analgesic compounds and various methods for their making and use. In one or more embodiments, he present invention is directed to a drug-loaded amino acid-based poly(ester urea) film for controlled local release of non-opioid analgesic compounds comprising an amino acid-based poly(ester urea) polymer or copolymer and a therapeutically effective amount of a non-opioid analgesic compound. In various embodiments, these amino acid-based poly(ester urea) polymers or copolymers will comprise a plurality of diester monomer units connected by a carboxyl group to form a poly(ester urea) (PEU) polymer.

### CROSS REFERENCE TO RELATED APPLICATIONS

This application claims the benefit of International application serial number PCT/US2021/016542 entitled “Drug-Loaded Amino Acid Based Poly(Ester Urea) Films for Controlled Local Release of Non-Opioid Analgesic Compounds,” filed Feb. 4, 2021, which claims the benefit of U.S. provisional patent application Ser. No. 62/969,919 entitled “Drug-Loaded Amino Acid Based Poly(Ester Urea) Films for Controlled Local Release of Non-Opioid Analgesic Compounds,” filed Feb. 4, 2020, both of which are incorporated herein by reference in their entirety.

### NAMES OF THE PARTIES TO A JOINT RESEARCH AGREEMENT

The present application stems from work done pursuant to Joint Research Agreements between the University of Akron of Akron, Ohio, Merck Sharp & Dohme Corp. of Rahway, New Jersey and 21^{st} Century Medical Technologies.

### FIELD OF THE INVENTION

One or more embodiments of the present invention relates to polymer-based drug delivery. In certain embodiments, the present invention relates to drug-loaded amino acid-based poly(ester urea) films for controlled local release of drugs.

### BACKGROUND OF THE INVENTION

Approximately 234 million surgeries are performed around the world annually, of which postoperative pain is reported in at least 75% of patients following any major procedure. Although a seemingly obvious side effect of any operation, proper pain management following a medical procedure is crucial to enhancement of patient recovery thereafter. Moreover, inadequate treatment of pain postoperatively can lead to additional medical and psychological Outcomes including chronic pain that may decrease the patient's quality of life, as well as causing increased medical costs. Despite advancements in current technology, pain management remains a challenge because treatment methods are not a one size fits all solution. Even under identical surgeries, management strategies are highly variable with large deviations required from person to person. This has led to physicians taking an overestimated approach when prescribing pain management drugs where patients can take analgesics “as needed”. While in principle this approach is fine, when the quantity and strength of prescribed drug vastly exceeds the amount required to achieve analgesia, the potential for drug abuse becomes prominent.

Much of the current solutions to post-surgical pain management involve the systemic distribution of analgesic, such as oral (PO) or intravenous (IV) dosage. Opioids are often used due to their high potency that is needed for proper bioavailability to the site of injury when orally dosed. Although PO and IV dosing are simple, practical methods, delivering these potent drugs or any drug systemically heightens potential for adverse effects, such as drowsiness, decreased cardiovascular function, and other effects on the central nervous system that can lead to addiction. Moreover, most strategies in shifting away from opioid use have focused on long-term prescriptions rather than outpatient prescriptions. The number of opioids prescribed varies widely but they are almost always prescribed in excess. This disproportionally leaves first time opioid users at risk for drug abuse with about 1 in 16 opioid-naïve patients becoming long-time users after surgery. Considering the validated risks associated with the current practices, post-operative analgesia alternatives are constantly emerging in the literature, though none have made significant headway and are still focused on systemic delivery of drugs.

One such solution are devices that afford more control over patient analgesia with the addition of a biomaterial matrix. To date, a variety of biomaterials have surfaced that exhibit controlled delivery of drugs, such as metal-organic compounds, natural polymers, and synthetic polymers. The intent of these materials is to extend the effect of the loaded medication while concomitantly achieving local delivery. Though all have their individual benefits, synthetic polymers offer the most practicality and the highest degree of tunability. There is much interest in resorbable synthetic materials for drug delivery as they can be used to provide sustained local drug release. Additionally, degradable materials are considered a temporary device which helps to limit regulatory hurdles often observed with permanent devices or those that require timely removal.

Poly(lactic-co-glycolic acid) (PLGA) has been extensively used in the biomedical field dating back to its introduction as an FDA approved product. Since then, PLGA has been researched for its ability to deliver analgesic compounds in the form of microparticles, films, and hydrogels with notable success and FDA approval in medicinal or medical products. Moreover, its bioresorbable nature has gotten it much attention as a temporary biomaterial in pain relief. However, several factors have precluded PLGA from expanding its market share in the drug release field. Primarily, acidosis induced inflammation of local soft tissue in vivo upon the degradation of PLGA poses an increased risk to some of these devices. Induction of this acidic environment also catalyzes the degradation of the polymer, thus creating an inconsistent and unpredictable release of active pharmaceutical ingredient (API). Therefore, it has only been implemented in small-scale devices with shortened timeframes, limiting its versatility.

A novel class of polymers that are making significant progress in the biomedical field are poly(ester urea)s (PEUs). Improving on previously observed shortcomings of PLGA and other polyesters, PEUs have a self-buffering effect attributed to the urea moiety which does not preclude their use as large, long-term implantable devices. As such, PEUs have been shown in vivo to have a limited inflammatory response through several months. PEUs also afford a great deal of flexibility in their chemical structure and therefore their mechanical properties by altering the amino acid side group and the diol chain length. Their use as a biomaterial has already been implemented in applications ranging from soft tissue repair to critical size defect bone scaffolds. Building on these other applications, PEUs can be reasonably tuned in chemical structure to obtain different release profiles from the matrix. Additionally, different matrix factors can also be altered to potentially change the release profile, such as drug-loading and thickness. As such, these devices could afford analgesia to a patient for a given period and can be modified for specific periods and dosages of pain relief.

Non-opioid active pharmaceutical ingredients (API) are commonly used as model compounds due to their practicality as analgesic components. For example, etoricoxib (ARCOXIA™) is a type of NSAID that is commonly used to treat arthritis and other musculoskeletal pain. Etoricoxib is highly selective in the inhibition of the cyclooxygenase (COX-2) enzyme. COX-2 aids in the production of prostaglandin which can be found at the site of injury and is a contributor to the associated pain and inflammation. Therefore, etoricoxib eases pain by blocking production of prostaglandin and has shown promise in clinical trials However, when prescribed orally, adverse effects have been reported including cardiovascular and gastrointestinal complications. A dose-dependent increased risk of edema and hypertension was observed for 90 mg etoricoxib dosed patients in comparison to diclofenac at 150 mg, though a decrease in gastrointestinal events was noted. Although approved for use in the United Kingdom, this increase in cardiovascular complications was a significant factor in formulations not gaining FDA approval in the United States. Introduction of etoricoxib into a polymer matrix could limit these side effects by providing localized drug delivery, and therefore improve the clinical outcomes.

What is needed in the art is a resorbable drug-loaded amino acid-based poly(ester urea) film with a tailorable release profile for controlled local release of non-opioid analgesic compounds for providing sustained pain relief to the patient.

### SUMMARY OF THE INVENTION

In one or more embodiments, the present invention provides a drug-loaded amino acid-based poly(ester urea) film for controlled local release of non-opioid analgesic compounds and various methods for their making and use. These films provide the benefit of local drug delivery (i.e. applied directly at the operative site) and would not require subsequent removal from the body. With introduction of an analgesic component into a polymer matrix, the release of the drug is controlled, offering sustained pain relief to the patient and limiting the need for prescription-based pain medication. Furthermore, changing the polymer composition, drug load, or film thickness allows for a tailorable release profile and thus will be beneficial for various drug release applications. Well-established mouse models of diabetic neuropathic pain including in vivo implantation of the drug-loaded amino acid-based poly(ester urea) films of the present invention showed effective relief of pain for more than 4 days post-implantation and efficacious local drug delivery. Overall, implementation of local drug delivery systems such as this could reduce the need for opioid prescriptions associated with current pain management strategies.

In a first aspect, the present invention is directed to a drug-loaded amino acid-based poly(ester urea) film for controlled local release of non-opioid analgesic compounds comprising: an amino acid-based poly(ester urea) polymer or copolymer; and a therapeutically effective amount of a non-opioid analgesic compound. In some of these embodiments, the amino acid-based poly(ester urea) polymer or copolymer comprises a plurality of diester monomer units, the diester monomer units comprising the residues of two amino acids separated by from 6 to 12 carbon atoms. In some embodiments, the diester monomer units comprise the residues of two valine molecules separated by from 6 to 12 carbon atoms.

In one or more embodiments, the drug-loaded amino acid-based poly(ester urea) film of the present invention includes any one or more of the above referenced embodiments of the first aspect of the present invention wherein the amino acid-based poly(ester urea) polymer or copolymer is a polymer having the formula:

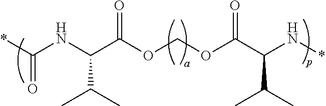

where a is an integer from 6 to 12, and p is an integer from 30 to 300.

In one or more embodiments, the drug-loaded amino acid-based poly(ester urea) film of the present invention includes any one or more of the above referenced embodiments of the first aspect of the present invention wherein the amino acid-based poly(ester urea) polymer or copolymer is a copolymer comprising: a plurality of first diester monomer units, the first diester monomer units comprising the residues of two amino acids separated by from 6 to 12 carbon atoms; and a plurality of second diester monomer units, the second diester monomer units comprising the residues of two amino acids separated by from 6 to 12 carbon atoms; wherein the amino acid residues in the first diester monomer units are different from the amino acid residues in the second diester monomer units.

In some of these embodiments, the plurality of first diester monomer units comprise the residues of two valine molecules separated by from 6 to 12 carbon atoms. In one or more embodiments, the drug-loaded amino acid-based poly(ester urea) film of the present invention includes any one or more of the above referenced embodiments of the first aspect of the present invention wherein the plurality of first diester monomer units comprise the reaction product of two amino acids and a C₆-C₁₂ linear diol. In one or more embodiments, the drug-loaded amino acid-based poly(ester urea) film of the present invention includes any one or more of the above referenced embodiments of the first aspect of the present invention wherein the plurality of first diester monomer units comprise the reaction product of two valine molecules and a C₆-C₁₂ linear diol. In one or more embodiments, the drug-loaded amino acid-based poly(ester urea) film of the present invention includes any one or more of the above referenced embodiments of the first aspect of the present invention wherein the plurality of second diester monomer units comprise the residues of two phenylalanine molecules separated by from 6 to 12 carbon atoms. In one or more embodiments, the drug-loaded amino acid-based poly(ester urea) film of the present invention includes any one or more of the above referenced embodiments of the first aspect of the present invention wherein the plurality of first diester monomer units comprise the reaction product of two amino acids and a C₆-C₁₂ linear diol. In one or more embodiments, the drug-loaded amino acid-based poly(ester urea) film of the present invention includes any one or more of the above referenced embodiments of the first aspect of the present invention wherein the plurality of second diester monomer units comprise the reaction product of two phenylalanine molecules and a C₆-C₁₂ linear diol.

In one or more embodiments, the drug-loaded amino acid-based poly(ester urea) film of the present invention includes any one or more of the above referenced embodiments of the first aspect of the present invention wherein the second diester monomer units comprise from about 10 mol % to about 50 mol %, preferably from about 20 mol % to about 40 mol %, and more preferably from about 20 mol % to about 30 mol % of the amino acid-based poly(ester urea) copolymer.

In one or more embodiments, the drug-loaded amino acid-based poly(ester urea) film of the present invention includes any one or more of the above referenced embodiments of the first aspect of the present invention wherein the amino acid-based poly(ester urea) copolymer is the reaction product of a first ion-protected diester monomer, a second ion-protected diester monomer, and a urea forming compound. In one or more embodiments, the drug-loaded amino acid-based poly(ester urea) film of the present invention includes any one or more of the above referenced embodiments of the first aspect of the present invention wherein the urea forming compound is selected from the group consisting of phosgene, diphosgene, and triphosgene.

In one or more embodiments, the drug-loaded amino acid-based poly(ester urea) film of the present invention includes any one or more of the above referenced embodiments of the first aspect of the present invention wherein the amino acid-based poly(ester urea) copolymer has a formula selected from:

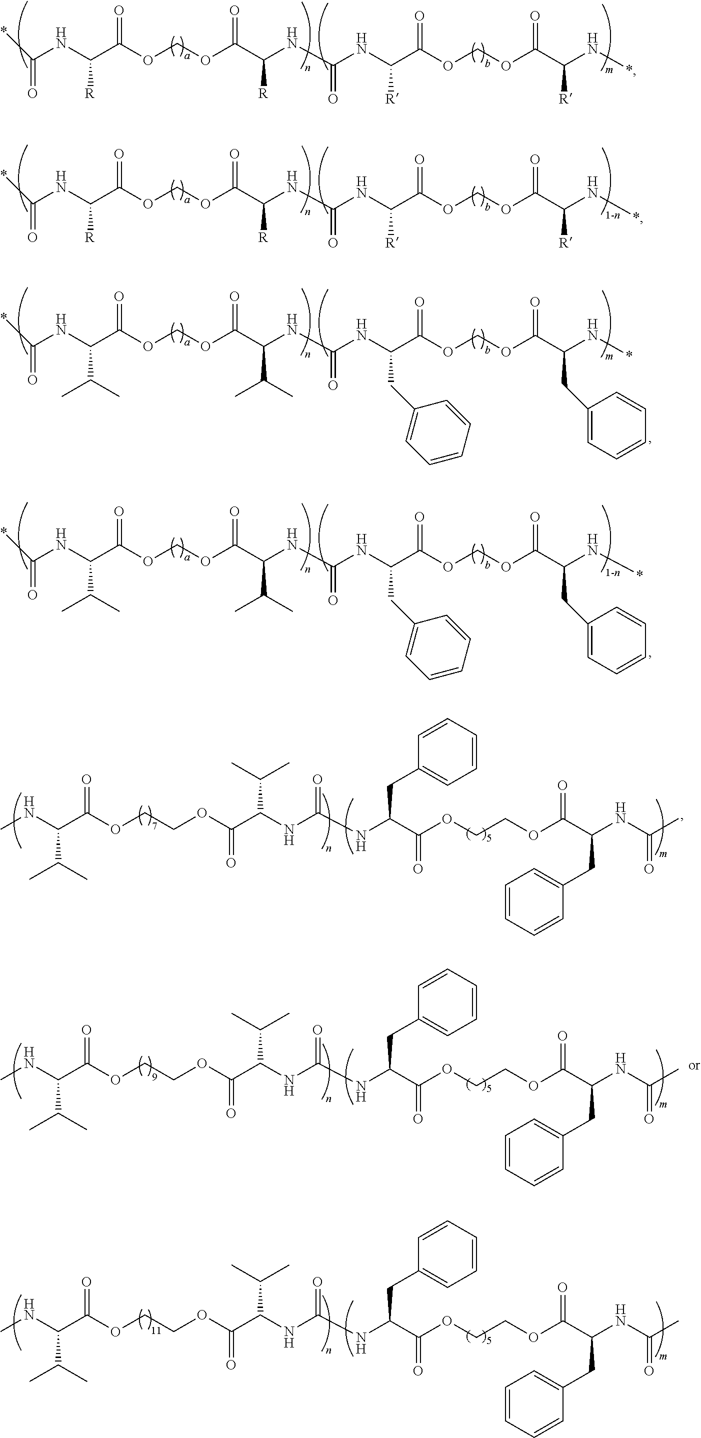

where a is an integer from 5 to 11; b is an integer from 5 to 11; R is —CH(CH₃)₂, —CH₃, —(CH₂)₃NHC(NH₂)C═NH, —CH₂CONH₂, —CH₂COOH, —CH₂SH, —(CH₂)₂COOH, —(CH₂)₂CONH₂, —NH₂, —CH₂C═CH—N═CH—NH, —CH(CH₃)CH₂CH₃, —CH₂CH(CH₃)₂, —(CH₂)₄NH₂, —(CH₂)₂SCH₃, —CH₂Ph, —CH₂OH, —CH(OH)CH₃, —CH₂—C═CH—NH-Ph, —CH₂-Ph-OH, or —CH₂C₆H₄OCH₂C₆H₅; R′ is CH₂Ph; n is a mole fraction from 0.9 to 0.6, and m is a mole fraction from 0.1 to 0.4.

In one or more embodiments, the drug-loaded amino acid-based poly(ester urea) film of the present invention includes any one or more of the above referenced embodiments of the first aspect of the present invention wherein the amino acid-based poly(ester urea) polymer or copolymer has a number average molecular weight of from about 10,000 Da to about 80,000 Da, preferably from about 10,000 Da to about 60,000 Da, and more preferably from about 20,000 Da to about 40,000 Da.

In one or more embodiments, the drug-loaded amino acid-based poly(ester urea) film of the present invention includes any one or more of the above referenced embodiments of the first aspect of the present invention wherein the non-opioid analgesic compound is selected from the group consisting of etoricoxib, bupivicaine, ropivacaine, lidocaine and pharmaceutically acceptable salts thereof. In one or more embodiments, the drug-loaded amino acid-based poly(ester urea) film of the present invention includes any one or more of the above referenced embodiments of the first aspect of the present invention wherein the non-opioid analgesic compound is etoricoxib or a pharmaceutically acceptable salt thereof. In one or more embodiments, the drug-loaded amino acid-based poly(ester urea) film of the present invention includes any one or more of the above referenced embodiments of the first aspect of the present invention wherein the non-opioid analgesic compound is substantially uniformly distributed throughout the amino acid-based poly(ester urea) polymer or copolymer. In one or more embodiments, the drug-loaded amino acid-based poly(ester urea) film of the present invention includes any one or more of the above referenced embodiments of the first aspect of the present invention comprising from about 1% to about 50%, preferably from about 10% to about 40%, and more preferably from about 20% to about 30% of the non-opioid analgesic compound by weight.

In one or more embodiments, the drug-loaded amino acid-based poly(ester urea) film of the present invention includes any one or more of the above referenced embodiments of the first aspect of the present invention having a thickness of from about 50 μm to about 500 μm In one or more embodiments, the drug-loaded amino acid-based poly(ester urea) film of the present invention includes any one or more of the above referenced embodiments of the first aspect of the present invention having a thickness of from about 10 μm to about 1000 μm.

In one or more embodiments, the drug-loaded amino acid-based poly(ester urea) film of the present invention includes any one or more of the above referenced embodiments of the first aspect of the present invention wherein the film continuously releases the non-opioid analgesic compound for from about 1 to about 187 days. In one or more embodiments, the drug-loaded amino acid-based poly(ester urea) film of the present invention includes any one or more of the above referenced embodiments of the first aspect of the present invention the film continuously releases the non-opioid analgesic compound for from about 3 to about 10 days. In one or more embodiments, the drug-loaded amino acid-based poly(ester urea) film of the present invention includes any one or more of the above referenced embodiments of the first aspect of the present invention wherein the non-opioid analgesic compound is released by diffusion. In one or more embodiments, the drug-loaded amino acid-based poly(ester urea) film of the present invention includes any one or more of the above referenced embodiments of the first aspect of the present invention having a Diffusivity Constant of from about 8.0×10⁻⁷ cm²/s to about 2.0×10⁻⁵ cm²/s.

In a second aspect, the present invention is directed to an amino acid-based poly(ester urea) film for controlled local release of etoricoxib comprising from about 50 to about 99 weight percent of an amino acid-based poly(ester urea) polymer or copolymer and from about 1 to about 50 weight percent etoricoxib or a pharmaceutically acceptable salt thereof. In some of these embodiments, the amino acid-based poly(ester urea) polymer or copolymer has the formula:

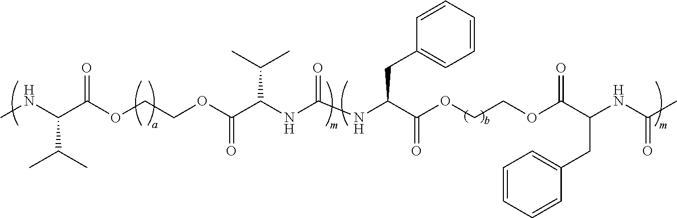

where a is an integer from 5 to 11; b is an integer from 5 to 11; n is a mole fraction from 90 to 60; and m is a mole fraction from 10 to 40.

In one or more embodiments, an amino acid-based poly(ester urea) film for controlled local release of etoricoxib of the present invention includes any one or more of the above referenced embodiments of the second aspect of the present invention wherein the amino acid-based poly(ester urea) polymer or copolymer has the formula:

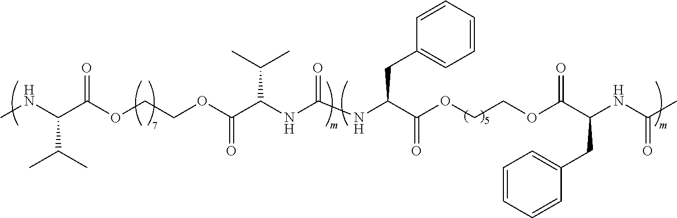

where n is a mole fraction from 90 to 70; and m is a mole fraction from 10 to 30.

In one or more embodiments, an amino acid-based poly(ester urea) film for controlled local release of etoricoxib of the present invention includes any one or more of the above referenced embodiments of the second aspect of the present invention wherein the amino acid-based poly(ester urea) polymer or copolymer has the formula:

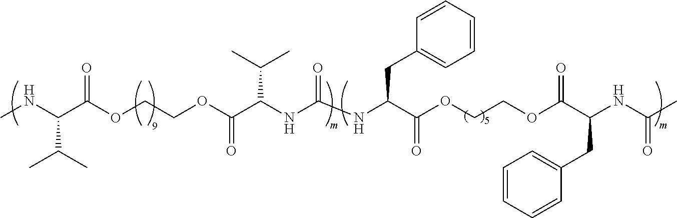

where n is a mole fraction from 90 to 70; and m is a mole fraction from 10 to 30.

In one or more embodiments, an amino acid-based poly(ester urea) film for controlled local release of etoricoxib of the present invention includes any one or more of the above referenced embodiments of the second aspect of the present invention wherein the amino acid-based poly(ester urea) polymer or copolymer has the formula:

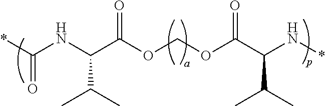

where a is an integer from 6 to 12 and p is an integer from about 30 to about 300.

In a third aspect, the present invention is directed to an amino acid-based poly(ester urea) film for controlled local release of etoricoxib comprising from about 50 to about 99 weight percent of an amino acid-based poly(ester urea) polymer or copolymer and from about 1 to about 50 weight percent bupivacaine or a pharmaceutically acceptable salt thereof. In one or more embodiments, an amino acid-based poly(ester urea) film for controlled local release of etoricoxib of the present invention includes any one or more of the above referenced embodiments of the third aspect of the present invention wherein the amino acid-based poly(ester urea) polymer or copolymer has the formula:

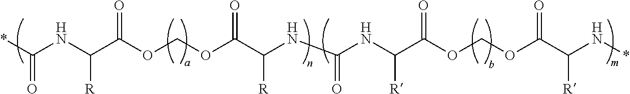

where a is an integer from 6 to 12; b is an integer from 6 to 12; n is a mole fraction from 90 to 60; and m is a mole fraction from 10 to 40. In one or more embodiments, an amino acid-based poly(ester urea) film for controlled local release of etoricoxib of the present invention includes any one or more of the above referenced embodiments of the third aspect of the present invention wherein the amino acid-based poly(ester urea) polymer or copolymer has the formula:

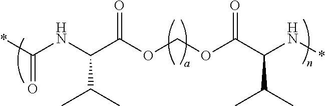

where a is an integer from 6 to 12 and n is an integer from about 30 to about 300.

In a fourth aspect, the present invention is directed to a drug-loaded amino acid-based poly(ester urea) film for controlled local release of non-opioid analgesic compounds comprising: an amino acid-based poly(ester urea) polymer or copolymer having the formula:

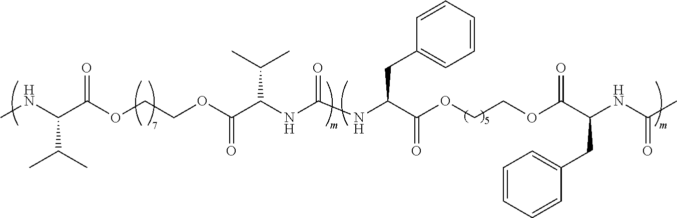

where n is a mole fraction from 0.9 to about 0.7 and m is a mole fraction from about 0.1 to about 0.3; and from about 20 wt % to about 40 wt % of etoricoxib or pharmaceutically acceptable salts thereof.

In a fifth aspect, the present invention is directed to a method for making the drug-loaded amino acid-based poly(ester urea) film described above comprising: dispersing or dissolving the amino acid-based poly(ester urea) polymer or copolymer and a non-opioid analgesic compound in a solvent for at least one of the amino acid-based poly(ester urea) polymer or copolymer and the non-opioid analgesic compound; mixing the amino acid-based poly(ester urea) polymer or copolymer/non-opioid analgesic compound dispersion or solution until the non-opioid analgesic compound is substantially homogeneously distributed throughout the amino acid-based poly(ester urea) polymer or copolymer; solvent casting the substantially homogeneous amino acid-based poly(ester urea) polymer or copolymer and non-opioid analgesic compound composition onto a substrate to form a non-opioid analgesic compound loaded amino acid-based poly(ester urea) polymer or copolymer film; allowing the non-opioid analgesic compound loaded amino acid-based poly(ester urea) polymer or copolymer film to dry under ambient conditions; and removing any remaining solvent by lyophilization or vacuum drying. In one or more embodiments, the method of the present invention includes any one or more of the above referenced embodiments of the fifth aspect of the present invention further comprising preparing the amino acid-based poly(ester urea) polymer or copolymer.

In one or more embodiments, the method of the present invention includes any one or more of the above referenced embodiments of the fifth aspect of the present invention wherein the amino acid-based poly(ester urea) polymer or copolymer has the formula:

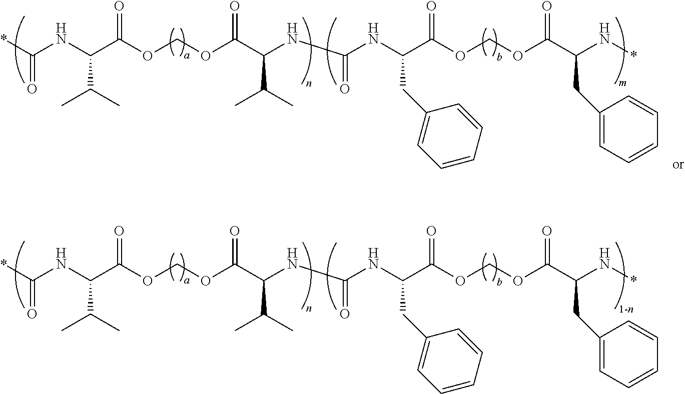

where a is an integer from 6 to 12; b is an integer from 6 to 12; n is a mole fraction from 90 to 60; and m is a mole fraction from 10 to 40. In one or more embodiments, the method of the present invention includes any one or more of the above referenced embodiments of the fifth aspect of the present invention In one or more embodiments, the method of the present invention includes any one or more of the above referenced embodiments of the fifth aspect of the present invention wherein the amino acid-based poly(ester urea) polymer or copolymer has the formula:

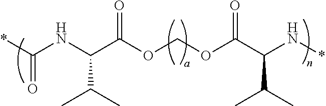

where a is an integer from 6 to 12, and n is an integer from 30 to 300.

In one or more embodiments, the method of the present invention includes any one or more of the above referenced embodiments of the fifth aspect of the present invention wherein the non-opioid analgesic compound is selected from the group consisting of etoricoxib, bupivacaine, ropivacaine, lidocaine and pharmaceutically acceptable salts thereof. In one or more embodiments, the method of the present invention includes any one or more of the above referenced embodiments of the fifth aspect of the present invention wherein the non-opioid analgesic compound is etoricoxib or a pharmaceutically acceptable salt thereof. In one or more embodiments, the method of the present invention includes any one or more of the above referenced embodiments of the fifth aspect of the present invention wherein both the amino acid-based poly(ester urea) polymer or copolymer and the non-opioid analgesic compound are soluble in the solvent B. In one or more embodiments, the method of the present invention includes any one or more of the above referenced embodiments of the fifth aspect of the present invention wherein the amino acid-based poly(ester urea) polymer or copolymer/non-opioid analgesic compound composition comprises from about 1% to about 50%, preferably from about 10% to about 40%, and more preferably from about 20% to about 30% of the non-opioid analgesic compound by weight.

In one or more embodiments, the method of the present invention includes any one or more of the above referenced embodiments of the fifth aspect of the present invention wherein the solvent is selected from acetone, 1,4-dioxane, methylene chloride, ethyl acetate, dimethyl sulfoxide, chloroform, methyl tetrahydrofuran, tetrahydrofuran and combinations thereof. In one or more embodiments, the method of the present invention includes any one or more of the above referenced embodiments of the fifth aspect of the present invention wherein the step of mixing comprises mixing the composition in an incubator at a temperature of from about 25° C. to about 45° C. for at least 24 hours.

In one or more embodiments, the method of the present invention includes any one or more of the above referenced embodiments of the fifth aspect of the present invention wherein the step of solvent casting is accomplished using a suitable Doctor blade apparatus. In some of these embodiments, the substrate is removable. In various embodiments, the substrate comprises polyethylene terephthalate (PET), polyethylene, 1,1,2,2-poly(tetrafluoroethylene), 1,1, poly(vinylidene difluoride) and combinations thereof. In one or more embodiments, the method of the present invention includes any one or more of the above referenced embodiments of the fifth aspect of the present invention wherein the step of allowing comprises allowing the non-opioid analgesic compound loaded amino acid-based poly(ester urea) polymer or copolymer film to dry under ambient conditions for at least 24 hours.

In a sixth aspect, the present invention is directed to a method for making the drug-loaded amino acid-based poly(ester urea) film described above comprising: dispersing or dissolving the amino acid-based poly(ester urea) polymer or copolymer having a formula selected from:

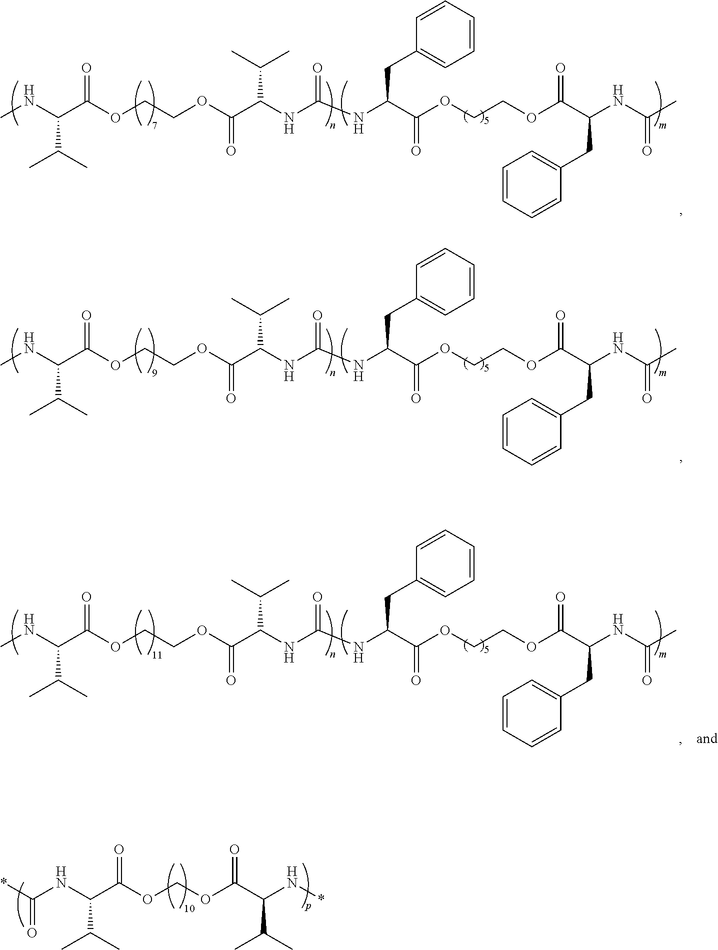

where n is a mole fraction from 0.9 to about 0.7 and m is a mole fraction from about 0.1 to about 0.3, and p is an integer from 30 to 300 and a non-opioid analgesic compound selected from the group consisting of etoricoxib, bupivacaine, ropivacaine, lidocaine and pharmaceutically acceptable salts thereof in a solvent selected from the group consisting of acetone, 1,4-dioxane, methylene chloride, ethyl acetate, dimethyl sulfoxide, chloroform, methyl tetrahydrofuran, tetrahydrofuran and combinations thereof, where the non-opioid analgesic compound is form 20% to 40% of the total weight of the amino acid-based poly(ester urea) polymer or copolymer and the non-opioid analgesic compound; mixing the amino acid-based poly(ester urea) polymer or copolymer/non-opioid analgesic compound dispersion or solution in an incubator at a temperature of from about 25° C. to about 45° C. for at least 24 hours to afford a substantially homogenous mixture of the non-opioid analgesic compound and the amino acid-based poly(ester urea) polymer or copolymer; solvent casting the substantially homogeneous amino acid-based poly(ester urea) polymer or copolymer and non-opioid analgesic compound composition onto a removable PET substrate using a Doctor blade apparatus; allowing the non-opioid analgesic compound loaded amino acid-based poly(ester urea) polymer or copolymer film to dry under ambient conditions for at least 24 hours to form a non-opioid analgesic compound loaded amino acid-based poly(ester urea) polymer or copolymer film; and removing any remaining solvent in the non-opioid analgesic compound loaded amino acid-based poly(ester urea) polymer or copolymer film by lyophilization or vacuum drying.

In a seventh aspect, the present invention is directed to a method for the localized treatment of a painful condition in a human or other mammal using the drug-loaded amino acid-based poly(ester urea) film described above comprising: identifying an area of the body of the human or other mammal where the painful condition exists; and inserting the drug-loaded amino acid-based poly(ester urea) film into the body of the human or other mammal at or in close proximity to the identified area, wherein the drug-loaded amino acid-based poly(ester urea) film is sized to deliver a therapeutically effective amount of a non-opioid analgesic compound for a predetermined time period. In one or more of these embodiments, the method further comprising preparing the drug-loaded amino acid-based poly(ester urea) film prior to the step of inserting.

In an eighth aspect, the present invention is directed to a method for the postoperative pain management in a human or other mammal after a surgical operation using the drug-loaded amino acid-based poly(ester urea) film described above comprising inserting the drug-loaded amino acid-based poly(ester urea) film into the body of the human or other mammal during the surgical operation, wherein the drug-loaded amino acid-based poly(ester urea) film is sized to deliver a therapeutically effective amount of a non-opioid analgesic compound for a predetermined time period. In some of these embodiments, the method further comprising the step of preparing the drug-loaded amino acid-based poly(ester urea) film prior to the step of inserting.

### BRIEF DESCRIPTION OF THE DRAWINGS

For a more complete understanding of the features and advantages of the present invention, reference is now made to the detailed description of the invention along with the accompanying figures in which:

FIG. 1 is a comparison of ¹HNMR spectra for: (A) V10, (B) P6₃₀V8₇₀, (C) 20% 1-PHE-6 P(1-VAL-8) P6₂₀V8₈₀, and (D) 1P6₁₀V8₉₀. Peaks are labeled with letters corresponding to the proton on the molecule to the left. Residual solvent peaks are identified. Integration was used to accurately determine the percent of phenylalanine (PHE) in the copolymer. The value is determined by calculating the percentage of the PHE (CH₂) peak (2.8 ppm) to the methyl peaks on valine (0.8 ppm) and is noted for each copolymer above the 2.8 ppm peak. Spectra was obtained from a 300 MHz Varian NMR spectrometer, and the DMSO solvent peak at 2.50 ppm was used as the reference.

FIGS. 2A-B are graphs showing size exclusion chromatography traces for P6₁₀V8₉₀, P6₂₀V8₈₀, P6₃₀V8₇₀, and V10 showing their molecular mass and molecular mass distribution (FIG. 2A) and thermogravimetric analysis (TGA) results for P6₁₀V8₉₀, P6₂₀V8_{b0}, P6₃₀V8₇₀, and V10 polymers showing their glass transition temperature (T_{g}) and demonstrating that the films are stable at physiological temperatures (FIG. 2B).

FIGS. 3A-I are a series of graphs showing cumulative etoricoxib release from PEU films in PBS solution in terms of label claim (%) (FIGS. 3A-C) and mass of API (mg) (FIGS. 3D-F). The films were also examined for elution differences via drug-load (FIGS. 3A, 3D, 3G), polymer composition (FIGS. 3B, E, H) and film thickness (FIGS. 3C, 3F, 3I). Release data was fit to a Higuchi model to quantify the rate at which etoricoxib is diffusing out of the film (FIGS. 3G-I).). Films with various thickness (FIGS. 3C, 3F, 3I) were tested for drug elution using a vial-shake method (100 rpm @37° C.), a less vigorous agitation rate than the rest of the groups tested.

FIGS. 4A-B are graphs showing release curves of etoricoxib-loaded PEU films using P6₃₀V8₇₀ (FIG. 4A) and V10 (FIG. 4B) at 20% (open shapes) and 40% (filled shapes) drug-loading. All films were tested in an Agilent 400-DS Dissolution Apparatus 7 at 37° C. with 40 DPM. Cumulative release (%) was calculated according to the remaining amount of etoricoxib in the film after it was retired from the study. Values for total release on day 7 are shown in the table below the graphs.

FIGS. 5A-C are graphs showing Higuchi model fitting for all tested films. The variables analyzed for diffusivity were drug-load (FIG. 5A), polymer composition (FIG. 5B), and film thickness (FIG. 5C). Diffusivity constants were calculated using the linear fit equation and used to estimate the time for 100% of the drug to be released. All linear fits had a Pearson square correlation coefficient of 0.97 or above. Statistical significance was determined by a one-way ANOVA with Tukey post hoc analysis of the diffusivity constants of each film. A value of p<0.05 was considered significant.

FIGS. 6A-C are graphs showing in vivo pharmacokinetic data for etoricoxib loaded PEU films according to one or more embodiment of the present invention. FIG. 6A plasma concentration of etoricoxib for the PEU films at time intervals over a period of 14 days. This data was deconvoluted to obtain cumulative milligrams released per timepoint (FIG. 6B). PEU films exhibited local drug delivery when compared to the plasma concentration at the conclusion of the study (FIG. 6C). The table (FIG. 6D) shows mean pharmacokinetic parameters (CV %) after administration of etoricoxib to Wister Han Rats; Comparison of etoricoxib exposure in plasma and tissue between oral (PO) and subcutaneous (SQ) delivery.

FIGS. 7A-C are individual rat plasma profiles for etoricoxib following SQ dosing of etoricoxib formulations with P6₁₀V8₉₀ (FIG. 7A), P6₃₀V8₇₀ (FIG. 7B), and V10 (FIG. 7C) polymeric films.

FIGS. 8A-C are schematic diagrams showing a perspective view (FIG. 8A), cross sectional view (FIG. 8B) and a front view (FIG. 8C) of a film fabrication using blade-coating where polymer solutions are poured into the well of a doctor blade that is then pulled by a drop down lever, leaving behind a film of the polymer solution on top of a PET substrate (FIG. 8A). The height of the doctor blade can be changed to give different film thickness by adjusting the screws on the top (FIGS. 8A-C).

FIG. 8D is an image showing one of the square sections of the dried film (4 cm²) that were cut-out to be tested for release.

FIGS. 9A-C are graphs showing one-way ANOVA with Tukey post hoc statistical analysis of the cumulative release of each film at day 7 of various PEU compositions (FIG. 9A), etoricoxib loads (FIG. 9B), and film thicknesses (FIG. 9C). A value of p<0.05 was considered significant.

FIGS. 10A-C are XRCT images of pre- and post-release films of V10 with 40% etoricoxib before the study (FIG. 10A), after the study (FIG. 10B), and without any etoricoxib for comparison (FIG. 10C).

FIGS. 11A-C are graphs showing the average amount of etoricoxib released per day from PEU films. Tested parameters were the effects of drug-load (FIG. 11A), polymer composition (FIG. 11B), and film thickness on total release of etoricoxib (FIG. 11C). Patterned bars indicate 20% etoricoxib loading and filled bars represent 40% etoricoxib loading.

FIG. 12 is a sample etoricoxib elution curve obtained using high-pressure liquid chromatography (HPLC) and standard operating conditions for content detection.

FIGS. 13A-D are DSC curves of physically mixed (FIGS. 13A and 13C) and blank controls (FIGS. 13B and 13D) etoricoxib-loaded PEU films at 20 (dotted line), 40 (dashed line), and 60 (solid line) weight percent drug loading. The uneven baseline around 100-120° C. is internal noise in the machine, as checked by running a blank pan. The first heating cycle of etoricoxib reveals a strong melting peak around 138° C. (FIG. 13A), which is then not apparent in the second heating cycle (FIG. 13B). Likewise, the physically mixed samples of PEU and etoricoxib reveal an initial melting peak for the drug, then none thereafter. This may suggest a change in etoricoxib phase once heated past its melting temperature. In drug-loaded films, crystalline peaks occur in the first heating cycle for 60% etoricoxib films (FIG. 13B, solid lines). In the second heating, all samples exhibit a glass transition, that increases with etoricoxib load for the copolymers, but decreases with etoricoxib for V10. In 60% films, it is thought that the observed second glass transition (arrow) is from a phase change from etoricoxib and in ones that do not show a second transition, that it is overlapped with the polymer transition.

FIGS. 14A-B are etoricoxib dissolution profiles from free drug and PEU films in mg (FIG. 14A) and percent (FIG. 14B). Film release was taken out to 7 days while free drug release was only taken out to three days as it reached a plateau around day 1. The dose of free drug into the USP 7 was about 6 mg (n=3), which is representative of the dose in these polymer films. Given the rapid dissolution of free etoricoxib in PBS, it is suggested that solubility was not a conflict in drug release from PEU films.

FIGS. 15A-C relate to the effects of etoricoxib-loaded film implantation on STZ-induced neuropathic pain. FIG. 15A is a schematic diagram showing implantation of etoricoxib-loaded PEU films according to one or more embodiment of the present invention around the sciatic nerve of mouse subjects; FIG. 15B is a graph showing paw withdrawal thresholds at intervals after STZ injection that illustrate that I.P. STZ (75 mg/kg) produced potent and persistent mechanical allodynia as indicated by decreased paw withdrawal threshold in comparison with the baseline (BL); and FIG. 15C is a graph showing paw withdrawal thresholds at intervals after implantation etoricoxib-loaded PEU film that indicates that a 40% (w/w) etoricoxib-loaded PEU film according to the present invention increased paw withdraw threshold in STZ-treated mice from day 1 to day 4 after the peri-sciatic implantation as compared to STZ BL. ###P<0.001 versus baseline (BL); ***p<0.001 and **p<0.01 versus STZ BL of each group. One-way ANOVA followed with Bonferroni's multiple comparisons test. n=5 mice in each group.

FIGS. 16A-D are histology images from P6₃₀V8₇₀ blank (FIG. 16A), P6₃₀V8₇₀ 40% etoricoxib-loaded (FIG. 16B), V10 blank (FIG. 16C), and V10 40% (FIG. 16D) etoricoxib-loaded subcutaneous hind thigh implants. Images are taken of the cross sectional areas of the tissue surrounding the implant and stained with H&E. * indicates where the PEU film is within the tissue sample.

FIG. 17 is a comparison of ¹HNMR spectra for monomer salts used for interfacial polymerization of poly(ester urea)s. Peaks are labelled accordingly. One spectra is labelled for M(1-VAL-X) as the chemical shift remains the same for the protons present when the diol length changes. Residual solvents peaks are noted and labelled. Chemical shifts are referenced to the DMSO solvent peak set to 2.50 ppm.

### DETAILED DESCRIPTION OF THE ILLUSTRATIVE EMBODIMENTS

The following is a detailed description of the disclosure provided to aid those skilled in the art in practicing the present disclosure. Those of ordinary skill in the art may make modifications and variations in the embodiments described herein without departing from the spirit or scope of the present disclosure. Unless otherwise defined, all technical and scientific terms used herein have the same meaning as commonly understood by one of ordinary skill in the art to which this disclosure belongs. The terminology used in the description of the disclosure herein is for describing particular embodiments only and is not intended to be limiting of the disclosure.

As set forth above, the present invention provides a drug-loaded amino acid-based poly(ester urea) film for controlled local release of non-opioid analgesic compounds and various methods for their making and use. These films provide the benefit of local drug delivery (i.e. applied directly at the operative site) and do not require subsequent removal from the body. With introduction of a non-opioid analgesic component into a polymer matrix, the release of the drug is controlled, offering sustained pain relief to the patient and limiting the need for prescription-based pain medication. Furthermore, changing the polymer composition, drug load, or film thickness allows for a tailorable release profile and it is believed that the drug-loaded amino acid-based poly(ester urea) films of the present invention will be beneficial for various drug release applications.

The following terms may have meanings ascribed to them below, unless specified otherwise. As used herein, the terms “comprising” “to comprise” and the like do not exclude the presence of further elements or steps in addition to those listed in a claim. Similarly, the terms “a,” “an” or “the” before an element or feature does not exclude the presence of a plurality of these elements or features, unless the context clearly dictates otherwise. Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein in the specification and the claim can be modified by the term “about.”

It should be also understood that the ranges provided herein are a shorthand for all of the values within the range and, further, that the individual range values presented herein can be combined to form additional non-disclosed ranges. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

All publications, patent applications, patents, and other references mentioned herein are expressly incorporated by reference in their entirety, which means that they should be read and considered by the reader as part of this text. That the document, reference, patent application, or patent cited in this text is not repeated in this text is merely for reasons of conciseness. In the case of conflict, the present disclosure, including definitions, will control. All technical and scientific terms used herein have the same meaning. Further, any compositions or methods provided herein can be combined with one or more of any of the other compositions and methods provided herein. The fact that given features, elements or components are cited in different dependent claims does not exclude that at least some of these features, elements or components maybe used in combination together.

In a first aspect, the present invention is directed to a drug-loaded amino acid-based poly(ester urea) film for controlled local release of non-opioid analgesic compounds comprising an amino acid-based poly(ester urea) polymer or copolymer and a therapeutically effective amount of a non-opioid analgesic compound. In various embodiments, these amino acid-based poly(ester urea) polymers or copolymers will comprise a plurality of diester monomer units connected by a carboxyl group to form a poly(ester urea) (PEU) polymer. The terms “diester monomer residue,” “amino acid-based diester monomer residue,” “residue of the [a] diester monomer” and “residue of the [an]amino acid-based diester monomer” are used interchangeably to refer to the part of a counter-ion protected amino acid-based diester monomer used to form the amino acid-based PEU polymer of copolymer of embodiments of the present invention that has been incorporated into the structure of the PEU.

In one or more embodiments, each of these diester monomer units will comprise the residues of two amino acids separated by from 6 to 12 carbon atoms. The amino acids forming the diester monomer units are not particularly limited, provided that the residual functional groups do not impeded the polymerization. The amino acids forming the diester monomer units may be either proteinogenic or non-proteinogenic. Proteinogenic amino acids include those amino acids that are incorporated into proteins during translation. Specific examples of proteinogenic amino acids include L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, and L-valine, L-selenocysteine, and L-pyrrolysine. Non-proteinogenic amino acids include those amino acids no coded for genetically. They may be prepared, for example, by post-translational modification. Also included in non-proteinogenic amino acids are those amino acids prepared synthetically by modifying or functionalizing an amino acid. Specific examples of functionalized amino acids may be found in PCT/US14/58264, which is incorporated herein by reference.

In some embodiments, the amino acids residues forming the diester monomer units of the amino acid-based poly(ester urea) polymer or copolymer of the drug-loaded amino acid-based poly(ester urea) films of the present invention may be residue of alanine (ala—A); arginine (arg—R); asparagine (asn—N); aspartic acid (asp—D); cysteine (cys—C); glutamine (gln—Q); glutamic acid (glu—E); glycine (gly—G); histidine (his—H); isoleucine (ile—I); leucine (leu—L); lysine (lys—K); methionine (met—M); phenylalanine (phe—F); serine (ser—S); threonine (thr—T); tryptophan (trp—W); tyrosine (tyr—Y); valine (val—V) or a combination thereof. In some embodiments, one or more of the diester monomer units forming the amino acid-based poly(ester urea) polymer or copolymer will comprise two valine residues. In some embodiments, one or more of the diester monomer units forming the amino acid-based poly(ester urea) polymer or copolymer will comprise two phenylalanine residues.

As set forth above, the two amino acids residues forming the diester monomer units of the amino acid-based poly(ester urea) polymer or copolymer will be separated from each other by from about 6 to about 12 carbon atoms, generally in the form of an alkylene group (i.e., CₙH₂ₙ) having from about 6 to about 12 CH₂ units. In some embodiments, the diester monomer units of the amino acid-based poly(ester urea) polymer or copolymer will be separated from each other by from about 6 to about 10 carbon atoms, in other embodiments, from about 6 to about 8 carbon atoms, in other embodiments, from about 8 to about 12 carbon atoms, and in other embodiments, from about 10 to about 12 carbon atoms. In some of these embodiments, the diester monomer units will comprise the residues of two valine molecules separated by from 6 to 12 carbon atoms. In some embodiments, one or more of the diester monomer units will comprise the residues of two phenylalanine molecules separated by from 6 to 12 carbon atoms.

In one or more embodiments, each of the diester monomer units will be the residue of an amino acid-based diester monomer used to form the amino acid-based poly(ester urea) polymer or copolymer. In one or more embodiments, each amino acid-based diester monomer will be the reaction product two amino acids and a C₆ to C₁₂ diol and will comprise two amino acid residues, as discussed above, along with the residue of the C₆ to C₁₂ diol used to form the amino acid-based diester monomer. In various embodiments, suitable C₆ to C₁₂ diol residues may include, without limitation, the residue of 1,6-hexanediol, 1,8-octanediol, 1,9-nonanediol, 1,10-decanediol, 1,11-undecanediol, or 1,12-dodecanediol.

The reaction of the polyol with the amino acid to create these amino acid-based polyester monomers can be achieved in any number of ways generally known to those of skill in the art. Generally, a condensation reaction at temperatures exceeding the boiling point of water involving a slight molar excess (˜2.1 eq.) of the acid relative to the hydroxyl groups is sufficient to enable the reaction to proceed.

In some embodiments, the amino acid-based diester monomers may be synthesized as shown in Scheme 1, below.

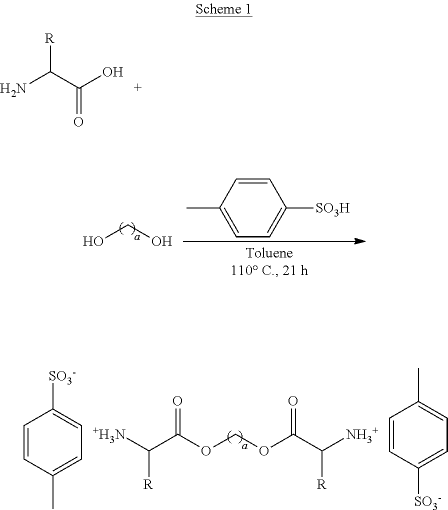

wherein R is an amino acid side chain and a is an integer from about 5 to about 12. In some embodiments, R may be any one or more of —CH₃, —(CH₂)₃NHC(NH₂)C═NH, —CH₂CONH₂, —CH₂COOH, —CH₂SH, —(CH₂)₂COOH, —(CH₂)₂CONH₂, —NH₂, —CH₂C═CH—N═CH—NH, —CH(CH₃)CH₂CH₃, —CH₂CH(CH₃)₂, —(CH₂)₄NH₂, —(CH₂)₂SCH₃, —CH₂Ph, —CH₂OH, —CH(OH)CH₃, —CH₂—C═CH—NH-Ph, —CH₂-Ph-OH, —CH(CH₃)₂. and —CH₂C₆H₄OCH₂C₆H₅. In some of these embodiments, a may be an integer from about 6 to about 12. In some of these embodiments, a may be an integer from about 8 to about 12. In some of these embodiments, a may be an integer from about 6 to about 10. In some of these embodiments, a may be an integer from about 6 to about 8. In some of these embodiments, a may be 5, 7, 9 or 11. In some other embodiments, a may be an integer from 1 to 20.

In the embodiments shown in Scheme 1 above, one or more amino acids, a linear diol having from 2 to 20 carbon atoms, and p-toluene sulfonic acid monohydrate are dissolved in toluene, heated to a temperature of about 110° C. and refluxed for about 21 hours to produce the di-p-toluene sulfonic acid salt of an amino acid-based diester monomer having two or more amino acid residues separated by from about 6 to about 12 carbon atoms, depending upon the polyol used. (See also, Examples 1-5). In some embodiments, the amino acid, polyol and acid may be dissolved in a suitable solvent such as toluene, DMF, and 1,4-paradioxane. One of ordinary skill in the art will be able to select a suitable solvent without undue experimentation.

In some other embodiments, a counter-ion protected amino acid-based polyester monomer according to one or more embodiments of the present invention may be synthesized as shown in Scheme 2, below.

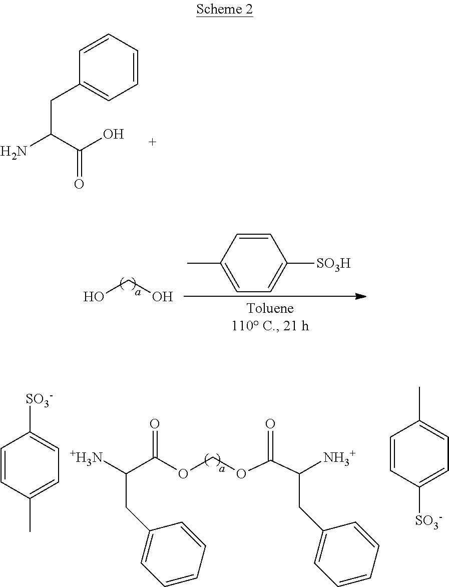

wherein a is an integer from about 1 to about 20. In some of these embodiments, a may be an integer from about 1 to about 15. In some of these embodiments, a may be an integer from about 1 to about 12. In some of these embodiments, a may be an integer from about 10 to about 20. In some of these embodiments, a may be an integer from about 4 to about 12. In some of these embodiments, a may be an integer from about 6 to about 12. In some of these embodiments, a may be 8.

In the embodiments shown in Scheme 2 above, L-phenylalanine, a linear diol having from 2 to 20 carbon atoms (a=2-20), and p-toluene sulfonic acid monohydrate are dissolved in toluene, heated to a temperature of from about 110° C. and refluxed for about 21 hours produce the di-p-toluene sulfonic acid salt of a L-phenylalanine based diester monomer having two phenylalanine residues separated by from about 2 to about 20 carbon atoms, depending upon the diol used. (See Scheme 2, above). In some of these embodiments, the solution may be heated to a temperature of from about 110° C. to about 112° C. In some of these embodiments, the solution may be refluxed for from about 20 hours to about 48 hours. In some of these embodiments, the solution may be refluxed for from about 20 hours to about 40 hours. In some of these embodiments, the solution may be refluxed for from about 20 hours to about 30 hours. In some embodiments, the di-p-toluene sulfonic acid salt of an L-phenylalanine based diester monomer, having two L-phenylalanine residues separated by from about 6 to about 12 carbon atoms, may be synthesized as set forth in Examples 3-5.

In various embodiments, the amino acid-based poly(ester urea) polymer or copolymer of the drug-loaded amino acid-based poly(ester urea) films may comprise a single type or more than one (generally two) type of diester monomer units. As will be apparent, in embodiments comprising a single type of diester monomer units, the amino acid-based poly(ester urea) polymer will be formed as a homopolymer of a single type of amino acid-based diester monomer with a urea forming material. Similarly, in embodiments comprising two or more types of type of diester monomer units, the amino acid-based poly(ester urea) polymer will be formed as a copolymer of two or more different types of amino acid-based diester monomers with a urea forming material. As used herein, the terms “urea forming material,” “PEU forming compound” and “PEU forming material” are used interchangeably to refer to a material capable of placing a carboxyl group between two amine groups, thereby forming a urea bond and may include, without limitation, triphosgene, diphosgene, or phosgene.

In some embodiments, the amino acid-based poly(ester urea) polymer or copolymer of the drug-loaded amino acid-based poly(ester urea) films will comprise the residue of first type of diester monomer units and a second type of monomer units. In one or more embodiments, the first type of diester monomer unit will have the formula:

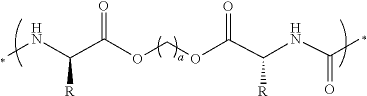

where a is an integer from 6 to 12 and R is —CH(CH₃)₂, —CH₃, —(CH₂)₃NHC(NH₂)C═NH, —CH₂CONH₂, —CH₂COOH, —CH₂SH, —(CH₂)₂COOH, —(CH₂)₂CONH₂, —NH₂, —CH₂C═CH—N═CH—NH, —CH(CH₃)CH₂CH₃, —CH₂CH(CH₃)₂, —(CH₂)₄NH₂, —(CH₂)₂SCH₃, —CH₂Ph, —CH₂OH, —CH(OH)CH₃, —CH₂—C═CH—NH-Ph, —CH₂-Ph-OH, or —CH₂C₆H₄OCH₂C₆H₅. In one or more embodiments, the first type of diester monomer unit will comprise the residues of two valine molecules separated by from 6 to 12 carbon atoms, as set forth above. In some embodiments, the first type of diester monomer unit will have the formula:

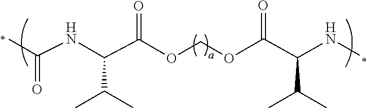

where a is an integer from 6 to 12.

Similarly, in one or more embodiments, the second type of diester monomer unit will have the formula:

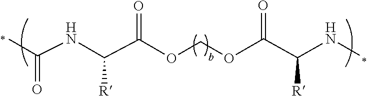

where b is an integer from 6 to 12 and R′ is —CH₂Ph. In some embodiments, the second type of diester monomer unit will have the formula:

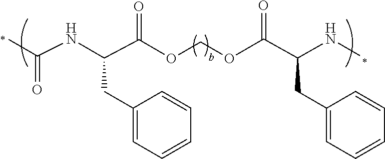

where b is an integer from 6 to 12.

In one or more embodiments, the second type of diester monomer unit will comprise the residues of two phenylalanine molecules separated by from 6 to 12 carbon atoms, as set forth above. In some of these embodiments, the second diester monomer units will comprise the reaction product of two phenylalanine molecules and a C₆-C₁₂ linear diol and will also include a carboxyl group provided by the PEU forming compound. In one or more of these embodiments, second diester monomer units will comprise from about 10 mol % to about 50 mol %, preferably from about 20 mol % to about 40 mol %, and more preferably from about 20 mol % to about 30 mol % of said amino acid-based poly(ester urea) copolymer.

In one or more embodiments, the amino acid-based poly(ester urea) polymer or copolymer will be formed by the interfacial homopolymerization of a counterion-protected diester monomer using a PEU forming compound, such as triphosgene. As will be apparent to those of skill in the art, if left unprotected, the ester bonds on the amino acid-based diester monomers will undergo transamidation, which can be prevented by protecting the amine groups on the amino acid-based diester monomers with one or more counter-ions. Accordingly, a suitable acid or other source of counter-ions may be added to the solution prior to or during formation of the diester monomer. One of ordinary skill in the art will be able to select a suitable counter-ion without undue experimentation. Materials capable of producing suitable protecting counter-ions may include, without limitation, p-toluene sulfonic acid monohydrate, chlorides, bromides, acetates. trifluoroacetate, or combinations thereof. In some embodiments, the acid used may be p-toluene sulfonic acid monohydrate. In some embodiments, the counterion-protected diester monomer will be the p-toluene sulfonic acid salt of the amino acid-based diester monomer.

In one or more embodiments, amino acid-based poly(ester urea) polymer or copolymer is a polymer having the formula:

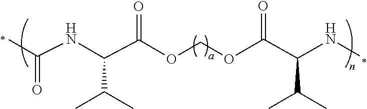

where a is an integer from 6 to 12, and n is an integer from 30 to 300. In some of these embodiments, a is an integer from 6 to 10, in other embodiments, from 6 to 8, in other embodiments, from 8 to 12, and in other embodiments, from 10 to 12. In some of these embodiments, n is an integer from 50 to 300, in other embodiments, from 100 to 300, in other embodiments, from 150 to 300, in other embodiments, from 200 to 300, in other embodiments, from 250 to 300, in other embodiments, from 30 to 250, in other embodiments, from 30 to 200, in other embodiments, from 30 to 150, in other embodiments, from 30 to 100, and in other embodiments, from 30 to 50.

In one or more embodiments, the amino acid-based poly(ester urea) polymer or copolymer of the drug-loaded amino acid-based poly(ester urea) films will be formed by the interfacial copolymerization of two or more different types of counterion-protected diester monomers using a PEU forming compound, such as triphosgene. As used herein, the term “interfacial polymerization” refers to polymerization that takes place at or near the interfacial boundary of two immiscible fluids. In some of these embodiments, the amino acid-based poly(ester urea) polymer or copolymer formed by the interfacial copolymerization of a first type of counterion-protected diester monomer and a second type of counterion-protected diester monomer, using a PEU forming compound, such as triphosgene. In some of these embodiments, the first type of diester monomer will comprise the reaction product of two amino acids and a C₆-C₁₂ linear diol and the second type of diester monomer will comprise the reaction product of two different amino acids and a C₆-C₁₂ linear diol.

In some of these embodiments, for example, the first type of counterion-protected diester monomer will comprise two valine molecules separated by from about 6 to about 12 carbon atoms and the second type of counterion-protected diester monomer will comprise two phenylalanine molecules separated by from about 6 to about 12 carbon atoms. In one or more of these embodiments, the first type of counterion-protected diester monomer will comprise the reaction product of two valine molecules and a C₆-C₁₂ linear diol and the second type of counterion-protected diester monomer will comprise the reaction product of two phenylalanine molecules and a C₆-C₁₂ linear diol. In some of these embodiments, the second diester monomer units comprise from about 10 mol % to about 50 mol %, preferably from about 20 mol % to about 40 mol %, and more preferably from about 20 mol % to about 30 mol % of said amino acid-based poly(ester urea) copolymer.

In various embodiments, the amino acid-based poly(ester urea) copolymer will have the formula:

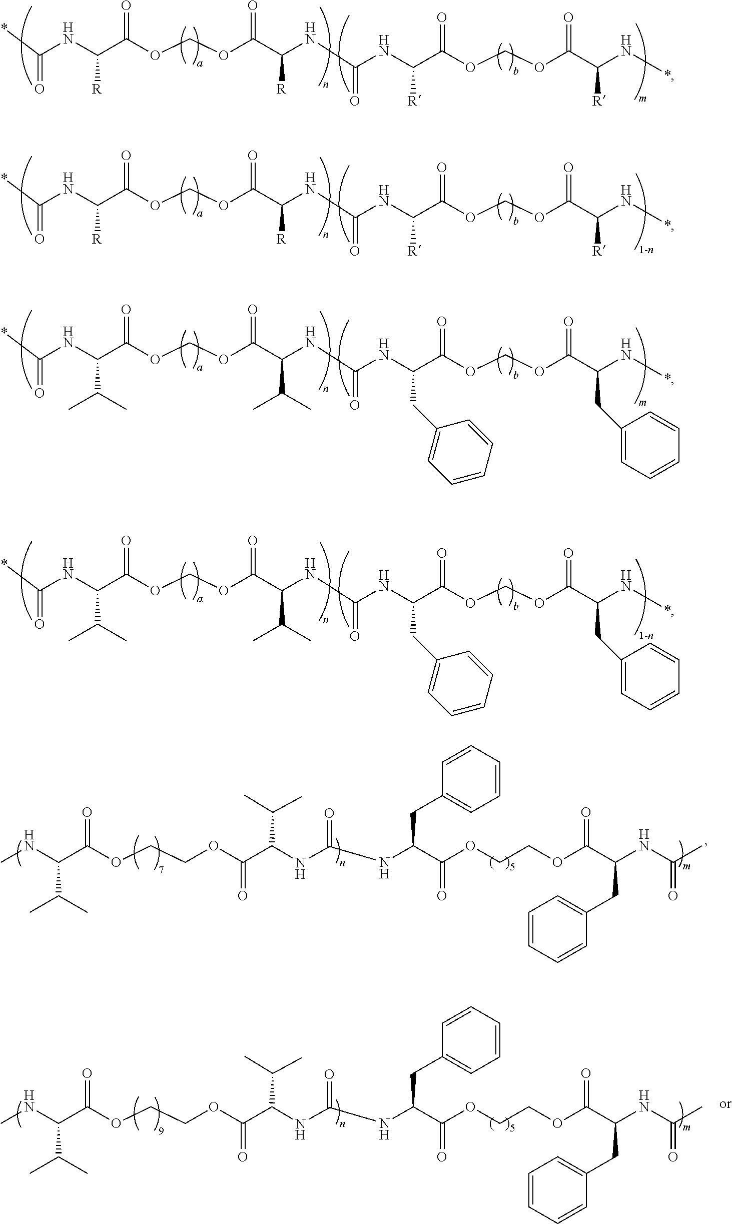

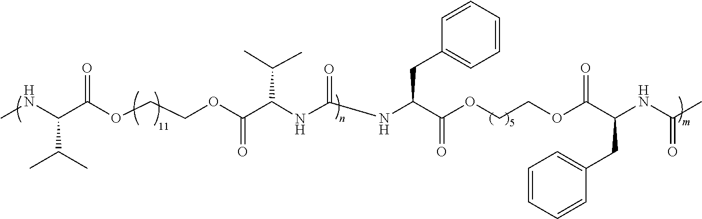

where a is an integer from 5 to 11; b is an integer from 5 to 11; R is —CH(CH₃)₂, —CH₃, —(CH₂)₃NHC(NH₂)C═NH, —CH₂CONH₂, —CH₂COOH, —CH₂SH, —(CH₂)₂COOH, —(CH₂)₂CONH₂, —NH₂, —CH₂C═CH—N═CH—NH, —CH(CH₃)CH₂CH₃, —CH₂CH(CH₃)₂, —(CH₂)₄NH₂, —(CH₂)₂SCH₃, —CH₂Ph, —CH₂OH, —CH(OH)CH₃, —CH₂—C═CH—NH-Ph, —CH₂-Ph-OH, or —CH₂C₆H₄OCH₂C₆H₅; R′ is CH₂Ph; n is a mole fraction from 0.9 to 0.6, and m is a mole fraction from 0.1 to 0.4.

In some of these embodiments, a may be an integer from about 6 to about 12. In some embodiments, a is an integer from 6 to 10, in other embodiments, from 6 to 8, in other embodiments, from 8 to 12, and in other embodiments, from 10 to 12. In some embodiments, b is an integer from 6 to 10, in other embodiments, from 6 to 8, in other embodiments, from 8 to 12, and in other embodiments, from 10 to 12. In some of these embodiments, a and b may each be 5, 7, 9 or 11. In some other embodiments, a and b may each be an integer from 1 to 20.

In some embodiments, the amino acid-based poly(ester urea) polymer or copolymer has the formula:

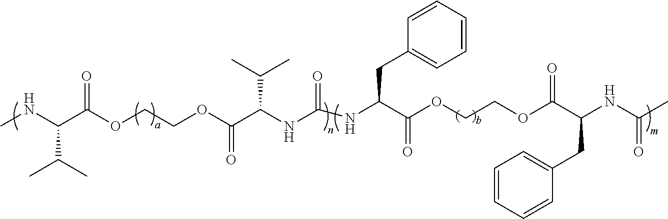

where a is an integer from 5 to 11; b is an integer from 5 to 11; n is a mole fraction from 90 to 60; and m is a mole fraction from 10 to 40.

In one or more embodiments, the amino acid-based poly(ester urea) polymer or copolymer has the formula:

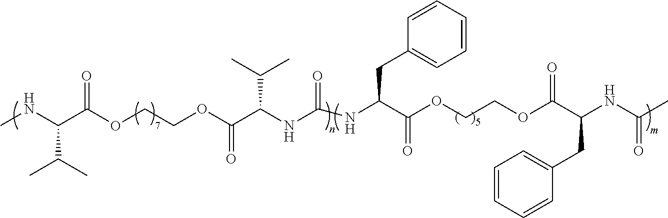

where n is a mole fraction from 90 to 70; and m is a mole fraction from 10 to 30.

In some embodiments, the amino acid-based poly(ester urea) polymer or copolymer has the formula:

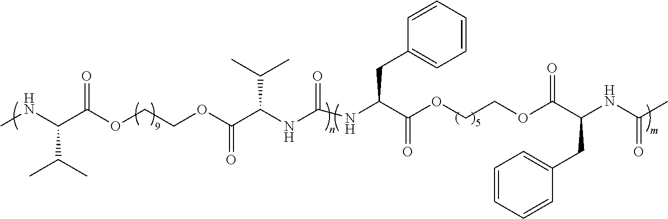

where n is a mole fraction from 90 to 70; and m is a mole fraction from 10 to 30.

In various embodiments, the amino acid-based poly(ester urea) polymer or copolymer will have a number average molecular weight of from about 10,000 Da to about 80,000 Da, preferably from about 10,000 Da to about 60,000 Da, and more preferably from about 20,000 Da to about 40,000 Da. In some embodiments, the amino acid-based poly(ester urea) polymer or copolymer will have a number average molecular weight of from about 10,000 Da to about 70,000 Da, in other embodiments, from about 10,000 Da to about 60,000 Da, in other embodiments, from about 10,000 Da to about 50,000 Da, in other embodiments, from about 10,000 Da to about 40,000 Da, in other embodiments, from about 20,000 Da to about 80,000 Da, in other embodiments, from about 30,000 Da to about 80,000 Da, in other embodiments, from about 40,000 Da to about 80,000 Da, in other embodiments, from about 50,000 Da to about 80,000 Da, and in other embodiments, from about 60,000 Da to about 80,000 Da. Here, as well as elsewhere in the specification and claims, individual range values can be combined to form additional non-disclosed ranges.

The amino acid-based poly(ester urea)s may be referred to herein by their amino acid residue, diol residue length, and monomer composition, if applicable. A monomer segment having two valine residues separated by the residue of a C10 diol may be referred to herein as “1-Val-10” or just “V10.” Similarly, a monomer segment having two valine residues separated by the residue of a C8 diol may be referred to herein as “1-VAL-8” or just “V8” and monomer segment having two phenylalanine residues separated by the residue of a C6 diol may be referred to herein as “1-PHE-16” or just “P6.” The homopolymer of one of these segments is simply referred to by the segment, so a homopolymer of V10 may be referred to herein as “P(1-VAL-10)” or “V10.” The nomenclature system used herein for co polymers is similar except all types of diester monomer segments are listed and their mole percent in the PEU placed in subscript following the diol residue chain length or, occasionally, before the first segment. So, a PEU co polymer having 10% P6 segments and 90% V8 segments would be referred to herein as “P6₁₀V8₉₀” or sometimes “10P6V8,” a PEU co polymer having 20% P6 segments and 80% V8 segments would be referred to herein as “P6₂₀V8₈₀” or sometimes “20P6V8,” and a PEU co polymer having 30% P6 segments and 70% V8 segments would be referred to herein as “P6₃₀V8₇₀” or sometimes “30P6V8.”

As set forth above, the drug-loaded amino acid-based poly(ester urea) films of the present invention will also comprise a non-opioid analgesic compound. As used herein, the term “non-opioid analgesic compound” is used to refer a compound that reduces the sensation of pain when administered to a human or other animal, but is not derived from the opium plant or synthesized to mimic the action of compounds derived from the poppy plant (e.g., morphine, heroine, codeine, oxycodone, hydrocodone, dihydromorphine, pethidine). In various embodiments, non-opioid analgesic compounds may include, without limitation, paracetamol (acetaminophen); nonsteroidal anti-inflammatory drugs (NSAIDs) (such as aspirin, ibuprofen, or naproxen); COX2 inhibitors (such as etoricoxib, rofecoxib, or celecoxib); psychotropic analgesic agents (such as ketamine, clonidine, or mexiletine); amide local anesthetics (such as, lidocaine, bupivacaine or ropivacaine), and combinations thereof. In one or more embodiments, the non-opioid analgesic compounds may include, without limitation, etoricoxib, bupivacaine, ropivacaine, lidocaine and pharmaceutically acceptable salts thereof. In some of these embodiments, the non-opioid analgesic compound is etoricoxib or a pharmaceutically acceptable salt thereof.

In various embodiments, the drug-loaded amino acid-based poly(ester urea) film of the present invention will comprise from about 1% to about 50%, preferably from about 10% to about 40%, and more preferably from about 20% to about 30% of said non-opioid analgesic compound by weight. In some embodiments, the drug-loaded amino acid-based poly(ester urea) film of the present invention will comprise from about 1% to about 50%, other embodiments, from about 10% to about 50%, in other embodiments, from about 20% to about 50%, in other embodiments, from about 30% to about 50%, in other embodiments, from about 40% to about 50%, in other embodiments, from about 1% to about 40%, in other embodiments, from about 1% to about 30%, in other embodiments, from about 1% to about 20%, and in other embodiments, from about 1% to about 10% non-opioid analgesic compound by weight. Here, as well as elsewhere in the specification and claims, individual range values can be combined to form additional non-disclosed ranges.

In these embodiments, the non-opioid analgesic compound will be substantially homogeneously distributed throughout the amino acid-based poly(ester urea) polymer or copolymer.

In some embodiments, the drug loaded amino acid-based poly(ester urea) films of the present invention comprise from about 50 to about 99 weight percent of an amino acid-based poly(ester urea) polymer or copolymer as described above and from about 1 to about 50 weight percent etoricoxib or a pharmaceutically acceptable salt thereof. In some other embodiments, the drug loaded amino acid-based poly(ester urea) film of the present invention will comprise from about 50 to about 99 weight percent of an amino acid-based poly(ester urea) polymer or copolymer as described above and from about 1 to about 50 weight percent bupivacaine or a pharmaceutically acceptable salt thereof.

In one or more embodiments, the drug-loaded amino acid-based poly(ester urea) film will comprise: an amino acid-based poly(ester urea) polymer or copolymer having the formula:

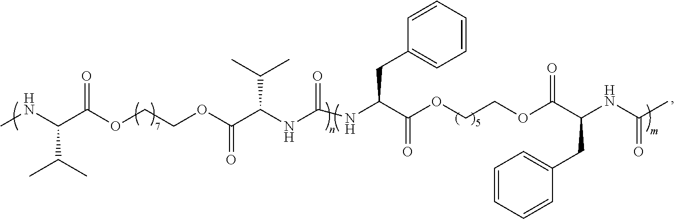

where n is a mole fraction from 0.9 to about 0.7 and m is a mole fraction from about 0.1 to about 0.3; and from about 20 wt % to about 40 wt % of etoricoxib or pharmaceutically acceptable salts thereof.

The size, shape, and thickness of the drug-loaded amino acid-based poly(ester urea) film of the present invention is not particularly limited. As will be apparent, however, the size, shape, and thickness of the film will affect the release rate and release profile of the film. In one or more embodiments, the drug-loaded amino acid-based poly(ester urea) films will have a thickness of from about 50 μm to about 500 μm. In some embodiments, the drug-loaded amino acid-based poly(ester urea) films will have a thickness of from about 100 μm to about 500 μm, in other embodiments, from about 150 μm to about 500 μm, in other embodiments, from about 200 μm to about 500 μm, in other embodiments, from about 300 μm to about 500 μm, in other embodiments, from about 400 μm to about 500 μm, in other embodiments, from about 50 μm to about 400 μm, in other embodiments, from about 50 μm to about 300 μm, in other embodiments, from about 50 μm to about 200 μm, and in other embodiments, from about 50 μm to about 100 μm.

As will be apparent to those of skill in the art, the release rate for the drug-loaded amino acid-based poly(ester urea) films of the present invention will depend upon a variety of factors, including the thickness of the film, the particular polymer or copolymer used, the drug loading, and the non-opioid analgesic compound being delivered. While the amino acid-based poly(ester urea) polymer or copolymers used to form these films are degradable and will break down into non-toxic byproducts, it is believed that drug release in these films occurs almost exclusively through diffusion of the non-opioid analgesic compound out of the amino acid-based poly(ester urea) polymer or copolymer prior to substantial degradation of the polymer. As will be apparent, the rate of drug release will depend upon the drug used, the drug loading, the polymer composition, and the film thickness. In some embodiments, etoricoxib-loaded amino acid-based poly(ester urea) films of the present invention may have a Diffusivity Constant from about 8.0×10⁻⁷ cm²/s to about 2.0×10⁻⁵ cm²/s.

In various embodiments, the drug-loaded amino acid-based poly(ester urea) films of the present invention will continuously release the non-opioid analgesic compound for from about 1 to about 190 days. In some embodiments, the drug-loaded amino acid-based poly(ester urea) films of the present invention will continuously release the non-opioid analgesic compound for from about 1 to about 150 days, in other embodiments, from about 1 days to about 120 days, in other embodiments, from about 1 days to about 90 days, in other embodiments, from about 1 days to about 60 days, in other embodiments, from about 1 days to about 30 days, in other embodiments, from about 1 days to about 14 days, in other embodiments, from about 4 days to about 190 days, in other embodiments, from about 10 days to about 190 days, in other embodiments, from about 21 days to about 190 days, in other embodiments, from about 30 days to about 190 days, in other embodiments, from about 60 days to about 190 days, and in other embodiments, from about 120 days to about 190 days. In some embodiments, the drug-loaded amino acid-based poly(ester urea) films of the present invention will continuously releases the non-opioid analgesic compound for from about 3 to about 10 days.

In another aspect, the present invention is directed to a method for making the drug-loaded amino acid-based poly(ester urea) film described above. An amino acid-based poly(ester urea) polymer or copolymer described above is selected for use in the film and synthesized or otherwise obtained. In various embodiments, the amino acid-based poly(ester urea) polymer or copolymer may be prepared as described above or using any suitable method known in the art. In some embodiments, the amino acid-based poly(ester urea) polymer or copolymer may be prepared as set forth in U.S. Pat. Nos. 9,745,414, 9,988,492, 10,280,261, 10,414,864, 10,537,660, US Published Application No. 2019/0167838 and 2020/368164, International Patent Publication Nos. WO 2020/226622 and WO 2020/226646, the disclosures of which are incorporated herein by reference in their entireties. Generally, the PEU homopolymers are synthesized with a single composition of monomer salt (1 eq.) is dissolved in water and triphosgene (0.4 eq) in chloroform is added. In some embodiments, excess sodium carbonate (2.3 eq.) or a similar compound is added to neutralize the HCl evolved from the triphosgene decomposition. The PEU copolymers are synthesized in a similar fashion, but with the addition of two compositions of monomer salts. Residual salt impurities are removed by reprecipitation of the polymer from acetone into hot water.

The non-opioid analgesic compound may be any of those described above and must likewise be selected and obtained. In some embodiments, the non-opioid analgesic compound may be etoricoxib, bupivacaine, ropivacaine, lidocaine or a pharmaceutically acceptable salt thereof. In some other embodiments, the non-opioid analgesic compound is etoricoxib or a pharmaceutically acceptable salt thereof.

Next, the amino acid-based poly(ester urea) polymer or copolymer and the non-opioid analgesic compound to be delivered are combined in a suitable mixing vessel with a solvent for at least one of the two ingredients. In some embodiments, both the amino acid-based poly(ester urea) polymer or copolymer and the non-opioid analgesic compound will be soluble in the solvent selected at the operating temperatures and pressures. It is strongly preferred, but not required, that both the amino acid-based poly(ester urea) polymer or copolymer and the non-opioid analgesic compound be soluble in the solvent selected as this provides for better and more uniform distribution of the non-opioid analgesic compound in the polymer or copolymer. In some other embodiments, however, only one component will be soluble in the solvent and the other dispersed throughout that solution.

The solvent used is not particularly limited and any suitable solvent may be used provided it does not react with, or otherwise degrade or denature, the amino acid-based poly(ester urea) polymer or copolymer or the non-opioid analgesic compound. Suitable solvents may include, without limitation, acetone, 1,4-dioxane, methylene chloride, ethyl acetate, dimethyl sulfoxide, chloroform, methyl tetrahydrofuran, tetrahydrofuran or combinations thereof. One of ordinary skill in the art will be able select a suitable solvent without undue experimentation.

The drug loading (defined here as the weight % of non-opioid analgesic compound relative to the total weight amino acid-based poly(ester urea) polymer or copolymer and the non-opioid analgesic compound) is not particularly limited, provided that the dispersion is uniform (homogeneous) and the drug remains amorphous and does not phase separate from the polymer. In some embodiments, the non-opioid analgesic compound will comprise from about 1% to about 50%, preferably from about 10% to about 40%, and more preferably from about 20% to about 30% of the total weight amino acid-based poly(ester urea) polymer or copolymer and a non-opioid analgesic compound. In some embodiments, the non-opioid analgesic compound will comprise from about 20% to about 40% of the total weight amino acid-based poly(ester urea) polymer or copolymer and a non-opioid analgesic compound.

The drug/polymer solution/dispersion is then mixed to ensure that the non-opioid analgesic compound is substantially homogeneously distributed throughout the amino acid-based poly(ester urea) polymer or copolymer. The drug/polymer solution/dispersion may be mixed by any conventional means including but not limited to magnetic stirring bars, impellers, and overhead mixers. In some embodiments, drug/polymer solution/dispersion may be mixed at a rate of from about 10 rpm to about 1000 rpm until non-opioid analgesic compound is substantially homogeneously distributed throughout the amino acid-based poly(ester urea) polymer or copolymer. The drug/polymer solution/dispersion may be mixed at a slightly elevated temperature to facilitate the substantially homogeneously distribution of the non-opioid analgesic compound throughout the amino acid-based poly(ester urea) polymer or copolymer. As will be obvious, this temperature should not be so high as to damage either the polymer of the non-opioid analgesic compound being used. In one or more embodiments, the drug/polymer solution/dispersion may be mixed at a temperature of from about 25° C. to about 45° C. As will also be apparent, the drug/polymer solution/dispersion will be mixed for a period of time sufficient to ensure that non-opioid analgesic compound is substantially homogeneously distributed throughout the amino acid-based poly(ester urea) polymer or copolymer. As used herein, the drug (API) is substantially homogeneously distributed in the throughout the amino acid-based poly(ester urea) polymer or copolymer when the concentration of the drug (API) is essentially the same (within about 10%) at any point in the polymer. In some embodiments, the drug/polymer solution/dispersion is mixed in an incubator at a temperature of from about 25° C. to about 45° C. for at least 24 hours to ensure that non-opioid analgesic compound is substantially homogeneously distributed throughout the amino acid-based poly(ester urea) polymer or copolymer.

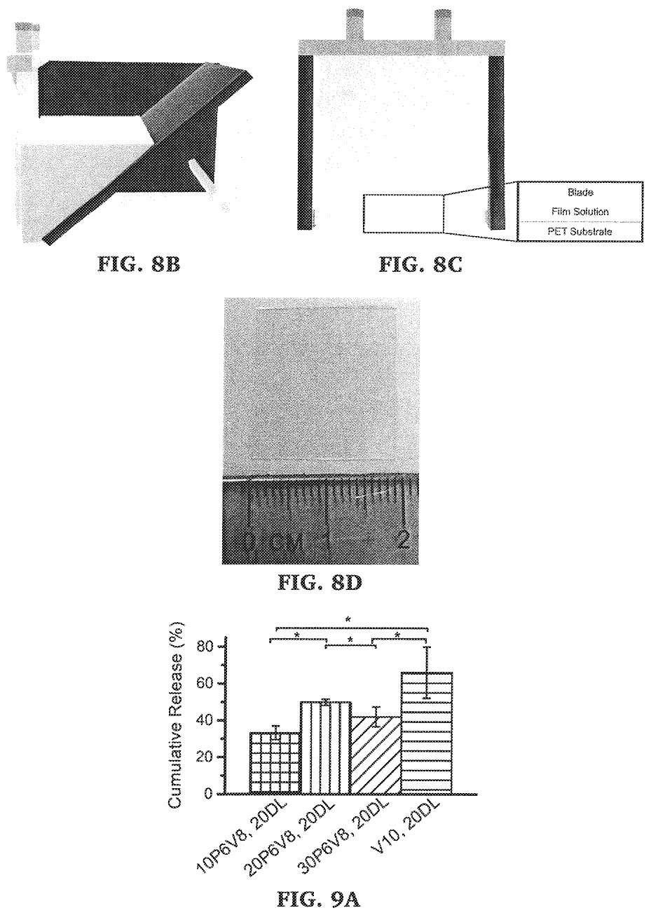

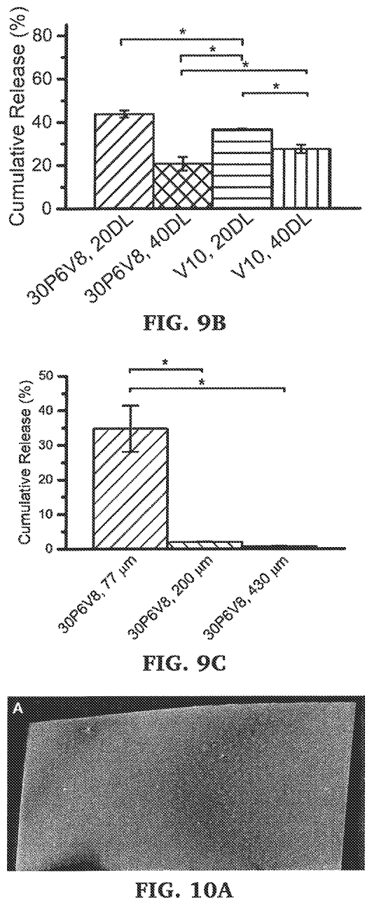

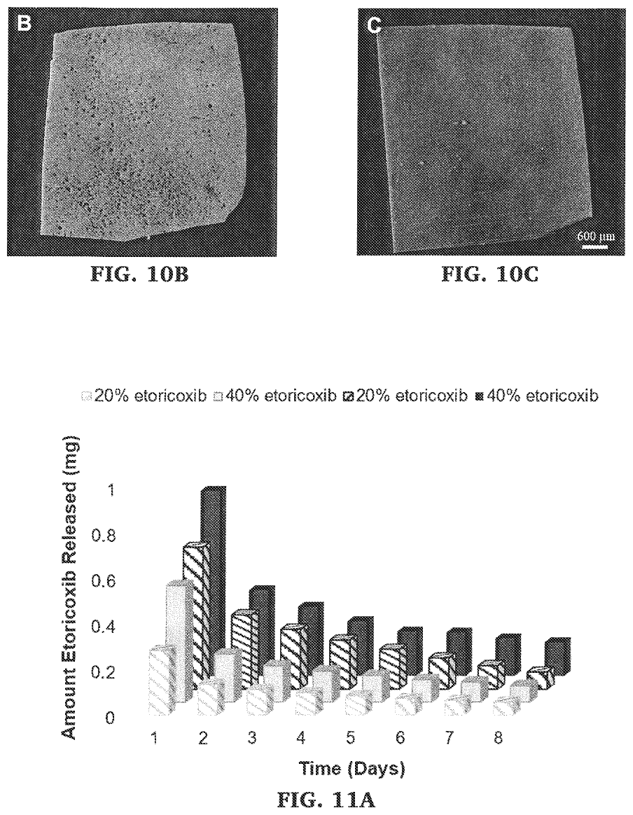

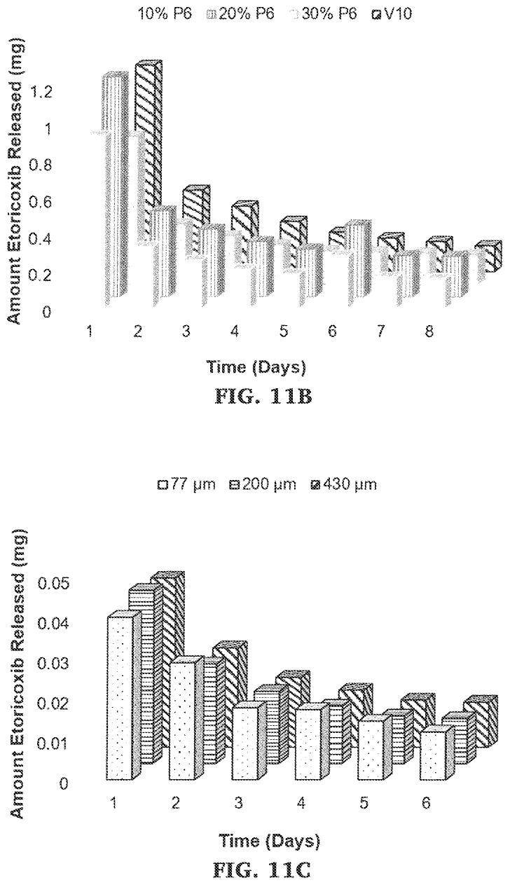

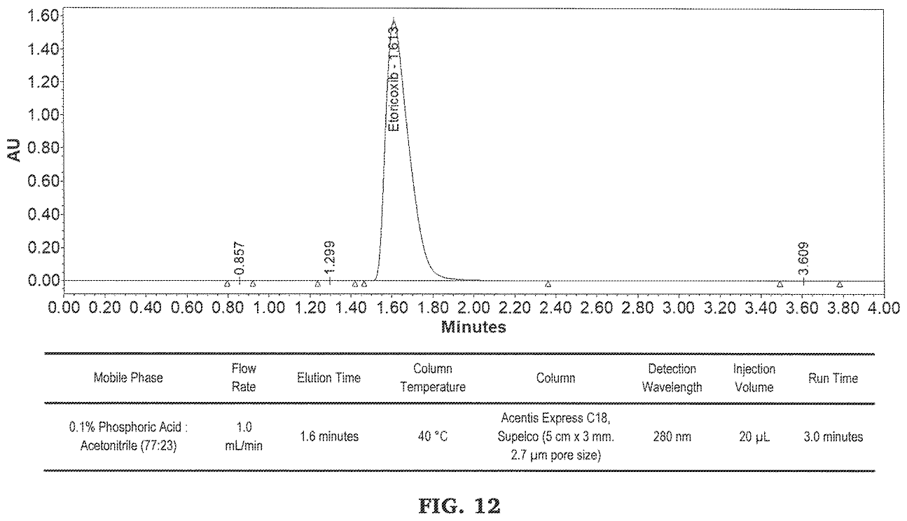

Finally. the substantially homogeneous amino acid-based poly(ester urea) polymer or copolymer and non-opioid analgesic compound composition is formed into a film. The substantially homogeneous amino acid-based poly(ester urea) polymer or copolymer and non-opioid analgesic compound composition may be formed into a film using any suitable method, but is preferably formed by solvent casting onto a suitable substrate using a Doctor blade as shown in FIGS. 8A-D. In some other embodiments, the film may be formed using conventional solvent casting, extrusion and/or injection molding processes. The substrate is not particularly limited and may include, without limitation, polyethylene terephthalate (PET), polyethylene, 1,1,2,2-poly(tetrafluoroethylene), 1,1, poly(vinylidene difluoride) and combinations thereof. In various embodiments, the substrate is removable to produce a free-standing film.

In one or more of these embodiments, a Doctor blade apparatus like the one shown in FIGS. 8A-C is placed on the substrate and the blade gap set at the desired thickness of the film, allowing for some shrinkage during the drying process. As shown in FIGS. 8A-C, the substantially homogeneous amino acid-based poly(ester urea) polymer or copolymer and non-opioid analgesic compound composition is loaded into the Doctor blade apparatus which is the moved along the substrate to form the film.

The film is then allowed to slowly dry under ambient conditions, preferably for at least 24 hours. While it is possible to dry the film more quickly, it has been found that doing so before the film has fully set can cause defects in the film. Once the film has fully set, any remaining water and solvent can be removed by lyophilization or vacuum drying.

In one or more of these embodiments, the present invention is directed to a method for making the drug-loaded amino acid-based poly(ester urea) film described above comprising the steps of: dispersing or dissolving said amino acid-based poly(ester urea) polymer or copolymer having a formula selected from:

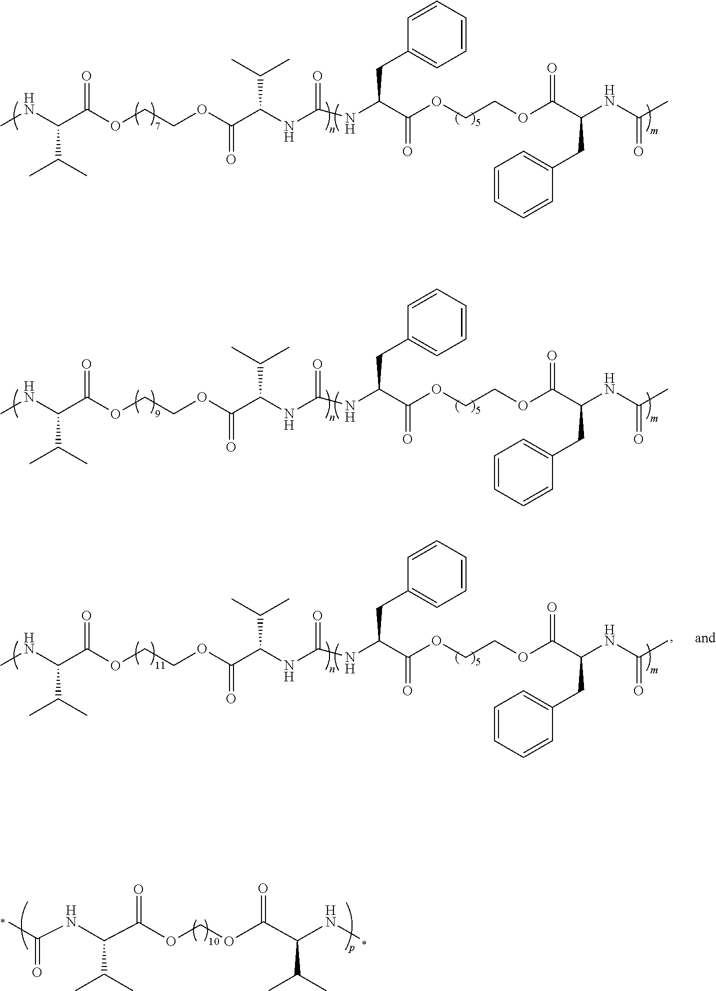

where n is a mole fraction from 0.9 to about 0.7 and m is a mole fraction from about 0.1 to about 0.3, and p is an integer from 30 to 300 and a non-opioid analgesic compound selected from the group consisting of etoricoxib, bupivacaine, ropivacaine, lidocaine and pharmaceutically acceptable salts thereof in a solvent selected from the group consisting of acetone, 1,4-dioxane, methylene chloride, ethyl acetate, dimethyl sulfoxide, chloroform, methyl tetrahydrofuran, tetrahydrofuran and combinations thereof, where said non-opioid analgesic compound is from 20% to 40% of the total weight of said amino acid-based poly(ester urea) polymer or copolymer and said non-opioid analgesic compound; mixing the amino acid-based poly(ester urea) polymer or copolymer/non-opioid analgesic compound dispersion or solution in an incubator at a temperature of from about 25° C. to about 45° C. and a speed of 80 rpm for at least 24 hours to afford a substantially homogenous mixture of said non-opioid analgesic compound and said amino acid-based poly(ester urea) polymer or copolymer; solvent casting the substantially homogeneous amino acid-based poly(ester urea) polymer or copolymer and non-opioid analgesic compound composition onto a removable PET substrate using a Doctor blade apparatus; allowing the non-opioid analgesic compound loaded amino acid-based poly(ester urea) polymer or copolymer film to dry under ambient conditions for at least 24 hours to form a non-opioid analgesic compound loaded amino acid-based poly(ester urea) polymer or copolymer film; and removing any remaining water or solvent in the non-opioid analgesic compound loaded amino acid-based poly(ester urea) polymer or copolymer film by lyophilization or vacuum drying.

In another aspect, the present invention is directed to a method for the localized treatment of a painful condition in a human or other mammal using the drug-loaded amino acid-based poly(ester urea) film described above comprising: identifying an area of the body of said human or other mammal where said painful condition exists; and inserting said drug-loaded amino acid-based poly(ester urea) film into the body of said human or other mammal at or in close proximity to said identified area, wherein said drug-loaded amino acid-based poly(ester urea) film is sized to deliver a therapeutically effective amount of a non-opioid analgesic compound for a predetermined time period. In various embodiments, drug-loaded amino acid-based poly(ester urea) film is sized to deliver a therapeutically effective amount of a non-opioid analgesic compound for from about 1 to about 190 days In some embodiments, drug-loaded amino acid-based poly(ester urea) film is sized to deliver an amount of the a non-opioid analgesic compound sufficient to ameliorate the painful condition for at least 3 days, in other embodiments, for at least 7 days, in other embodiments, for at least 10 days, in other embodiments, for at least 14 days, in other embodiments, for at least 21 days, in other embodiments, for at least 28 days, in other embodiments, for at least 60 days, and in other embodiments, for at least 90 days.

In still another aspect, the present invention is directed to a method for the postoperative pain management in a human or other mammal after a surgical operation using the drug-loaded amino acid-based poly(ester urea) film described herein comprising inserting the drug-loaded amino acid-based poly(ester urea) film into the body of said human or other mammal during the surgical operation, wherein the drug-loaded amino acid-based poly(ester urea) film is sized to deliver a therapeutically effective amount of a non-opioid analgesic compound for a predetermined time period. In some embodiments, drug-loaded amino acid-based poly(ester urea) film is sized to deliver an amount of the a non-opioid analgesic compound sufficient to ameliorate the painful condition for at least 3 days, in other embodiments, for at least 7 days, in other embodiments, for at least 10 days, in other embodiments, for at least 14 days, in other embodiments, for at least 21 days, in other embodiments, for at least 28 days, in other embodiments, for at least 60 days, and in other embodiments, for at least 90 days.

### Experimental

To evaluate and further reduce the drug-loaded amino acid-based poly(ester urea) films of the present invention to practice, various etoricoxib loaded amino acid-based poly(ester urea) films were prepared and tested. In these experiments, an economical and efficient blade-coating technique was utilized that affords control over the film dimensions (e.g. thickness, shape, and size) to attain drug-loaded poly(ester urea) (PEU) matrices with various drug-loads and polymer compositions. Considering all these variables, it is believed that patient or property specific films are possible. It is believed that the amino acid-based poly(ester urea) films of the present invention will be advantageous as post-operative implants for direct analgesia at the surgical site to limit the need for oral prescriptions. The PEU polymers used were selected for their previous indications as a material that did not produce notable inflammation in vivo as well as for their mechanical and physical properties. In these experiments, little to no inflammation of the implantation site was noted after histological analysis. Furthermore, the release of etoricoxib followed a continuous curve, indicating that no acidic environment was created in the soft tissue, therefore not producing erratic release of drug.

The amino acid-based poly(ester urea)s were synthesized using an interfacial polymerization of the respective monomer salts and triphosgene to produce homopolymer and copolymers as outlined in Scheme 3, below.

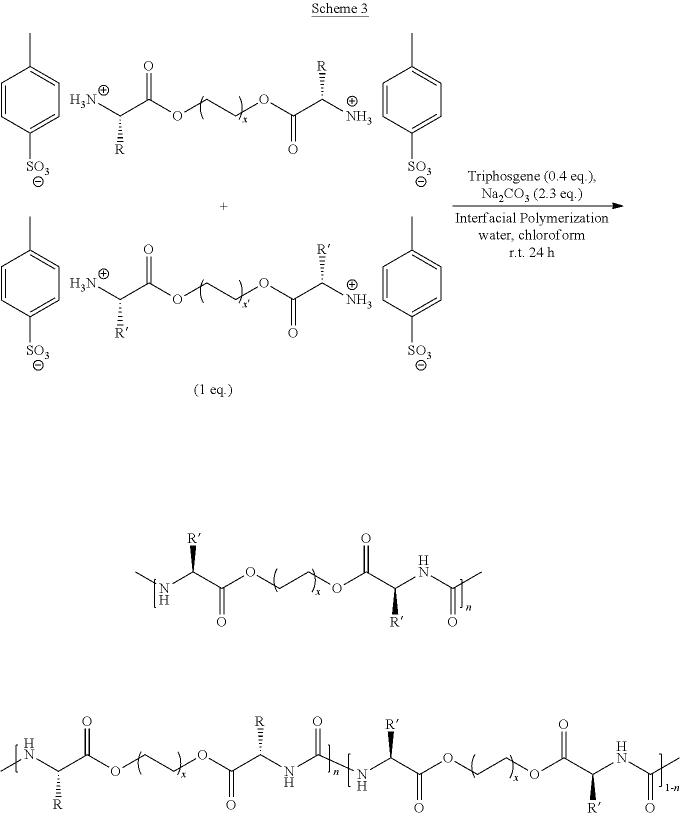

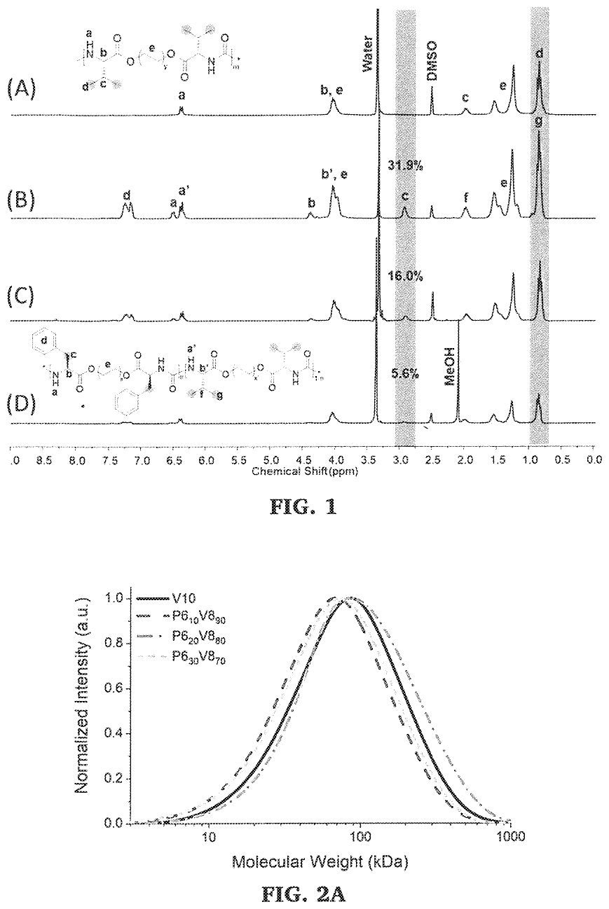

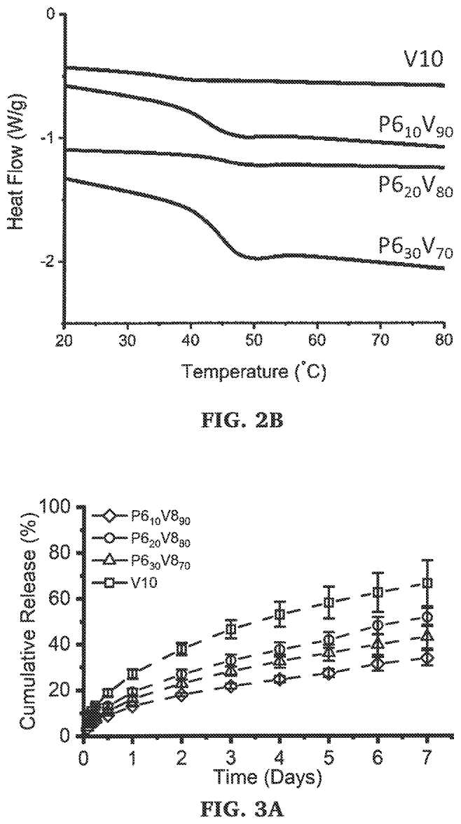

P(1-VAL-10) and random copolymers of 1-PHE-6 and 1-VAL-8 (10, 20 and 30 weight percent) structures were confirmed and checked for purity using ¹H NMR spectroscopy (FIG. 1). The stoichiometry of 1-PHE-6 in the backbone was determined by integration and division of the methylene and the methyl resonances and are highlighted on the NMR spectra shown In FIG. 1. The molecular masses varied slightly, and were considered, if necessary, to understand the drug release (FIG. 2A). Glass transition temperatures (T_{g}) were invariant with stoichiometry and amino acid composition in the backbone (FIG. 2B). Glass transition temperature (Table 1) and degradation temperatures demonstrated that the films are stable at physiological temperatures.

The general properties of the amino acid-based PEU polymers used in these experiments are set forth in Table 1, below.

TABLE 1
Polymer characterization data

| | M_{w} | Mₙ | | T_{g} | T_{d} |
|---|---|---|---|---|---|
| Polymer | (kDa) | (kDa) | Ðₘ | (° C.) | (° C.) |
| 10% 1-PHE-6 P(1-VAL-8) | 107 | 68 | 1.6 | 44 | 245 |
| 20% 1-PHE-6 P(1-VAL-8) | 88 | 56 | 1.6 | 57 | 278 |
| 30% 1-PHE-6 P(1-VAL-8) | 104 | 52 | 2.0 | 45 | 263 |
| P(1-VAL-10) | 216 | 143 | 1.5 | 42 | 275 |

Molecular mass distributions were obtained using SEC, glass transitions were estimated using DSC and degradation temperatures were obtained using TGA.

For film fabrication, an efficient blade-coating technique was utilized that enabled control over the film dimensions (e.g. thickness, shape, and size) to attain drug-loaded poly(ester urea) (PEU) matrices with various drug-loads and polymer compositions. Polymer and drug were dissolved in acetone to produce a substantially homogeneous transparent solution, which as then used to fabricate a solid film upon solvent evaporation. Considering the precise control of film variables, it is believed that patient specific drug formulations are possible. Various embodiments of the present invention could be advantageous for use as post-operative implants for direct analgesia at the surgical site to limit the need for oral prescriptions. As set forth above, the polymers used were selected as they did not induce notable inflammation in vivo as well as for their specific mechanical and physical properties. Furthermore, it was found that the release of etoricoxib from these etoricoxib loaded PEU films followed a continuous profile, indicating that matrix swelling and hydrolytic degradation of the PEU did not contribute significantly to the release. It is believed that this mechanism enhances the predictability of etoricoxib release from these films.

### Concentration Determination of Etoricoxib Films.

In this set of experiments, etoricoxib loaded PEU films were made by solvent casting with a Doctor blade as described above (see also, Example 8, below), using P(1-VAL-10), 10% 1-PHE-6 P(1-VAL-8), 20% 1-PHE-6 P(1-VAL-8) and 30% 1-PHE-6 P(1-VAL-8) polymers at 20 wt % and 40 wt % loadings of etoricoxib. Punches from each blade-coated film were measured and dissolved as described in Example 9 below to test for etoricoxib content via high-pressure liquid chromatograph (HPLC). The amount obtained from this procedure was assumed to be the actual etoricoxib amount. Theoretical values for etoricoxib content were calculated according to the mass of the film and the intended drug-load. It should be noted that this calculation assumes a homogeneous distribution of drug in the film. The predicted value was then compared to the actual etoricoxib amount calculated to quantify the accuracy of the theoretical method. See, Tables 2 and 3, below.

An analysis of the average drug content per surface area in film punches of P(1-VAL-10) (V10) and 30% 1-PHE-6 P(1-VAL-8) (P6₃₀V8₇₀) at drug loadings of 20 and 40 weight percent with a theoretical drug loading of 20 and 40 weight percent was conducted to determine the actual Etoricoxib content. The results are reported on Table 2. Content determination of etoricoxib in films tested for release. Following the release period, films were dissolved in a mixture of THF/Ethanol and analyzed for their remaining etoricoxib content (HPLC). Theoretical (predicted based on the weight of the film and the drug loading) values were compared to the actual amount of etoricoxib calculated throughout the duration of the study and with the result from the dissolved film. The accuracy of the theoretical predicted value was determined by obtaining a percentage of the theoretical drug load versus the actual drug load. It is believed that the overestimations can be attributed to aggregation of drug in the specific area that was cut out. Two films from each set were used in this measurement and the other was used for post-release micrograph surface morphology analysis.

TABLE 2

| | Drug Load | Weight of Film | Theoretical Drug | Actual Drug | Theoretical |
|---|---|---|---|---|---|
| Polymer | (wt. %) | (mg) | Weight (mg) | Weight (mg) | Accuracy (%) |
| P(1-VAL-10)* | 20 | 25.3 ± 7.5 | 5.1 ± 1.5 | 4.7 ± 1.8 | 109.9 ± 9.3 |
| P(1-VAL-10)* | 40 | 28.3 ± 6.8 | 11.3 ± 2.7 | 10.6 ± 2.2 | 106.8 ± 3.1 |
| 30% 1-PHE-6 P(1-VAL-8)* | 20 | 16.6 ± 2.2 | 4.5 ± 0.4 | 3.9 ± 0.1 | 110.7 ± 19.6 |
| 30% 1-PHE-6 P(1-VAL-8)* | 40 | 19.0 ± 1.8 | 7.6 ± 0.7 | 5.8 ± 0.6 | 131.5 ± 1.9 |

*indicates samples sets only containing n = 2, all other sets were n = 3.

An analysis of the average drug content per surface area in film punches of 10% 1-PHE-6 P(1-VAL-8) (P6₁₀V8₉₀), 20% 1-PHE-6 P(1-VAL-8) (P6₂₀V8₈₀), 30% 1-PHE-6 P(1-VAL-8) (P6₃₀V8₇₀) and (P(1-VAL-10) (V10) with a theoretical drug loading of 20 weight percent was also conducted to determine the actual content of Etoricoxib. The results are reported on Table 3. Theoretical (predicted based on the weight of the film and the drug loading) values were compared to the actual amount of etoricoxib calculated according to HPLC analysis. The accuracy of the theoretical predicted value was determined by obtaining a percentage of the theoretical drug load versus the actual drug load.

TABLE 3

| | Drug Load | Weight of | Theoretical Drug | Actual Drug | Theoretical |
|---|---|---|---|---|---|
| Polymer | (wt. %) | Film (mg) | Weight (mg) | Weight (mg) | Accuracy (%) |
| 10% 1-PHE-6 P(1-VAL-8) | 20 | 38.0 ± 7.6 | 7.6 ± 1.5 | 7.6 ± 1.9 | 100.5 ± 9.5 |
| 20% 1-PHE-6 P(1-VAL-8) | 20 | 27.0 ± 1.4 | 5.4 ± 0.3 | 7.5 ± 2.1* | 77.0 ± 20.2* |
| 30% 1-PHE-6 P(1-VAL-8) | 20 | 23.8 ± 5.3 | 4.8 ± 1.1 | 4.0 ± 1.0 | 120.8 ± 10.1 |
| P(1-VAL-10) | 20 | 18.9 ± 6.8 | 3.8 ± 1.4 | 3.0 ± 0.6 | 122.0 ± 19.0 |

*indicates samples sets only containing n = 2, all other sets were n = 3.

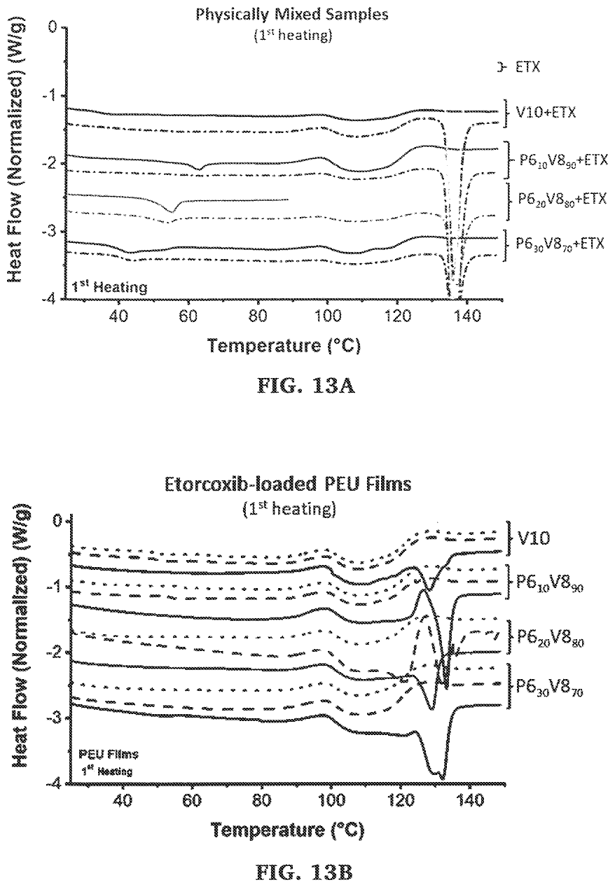

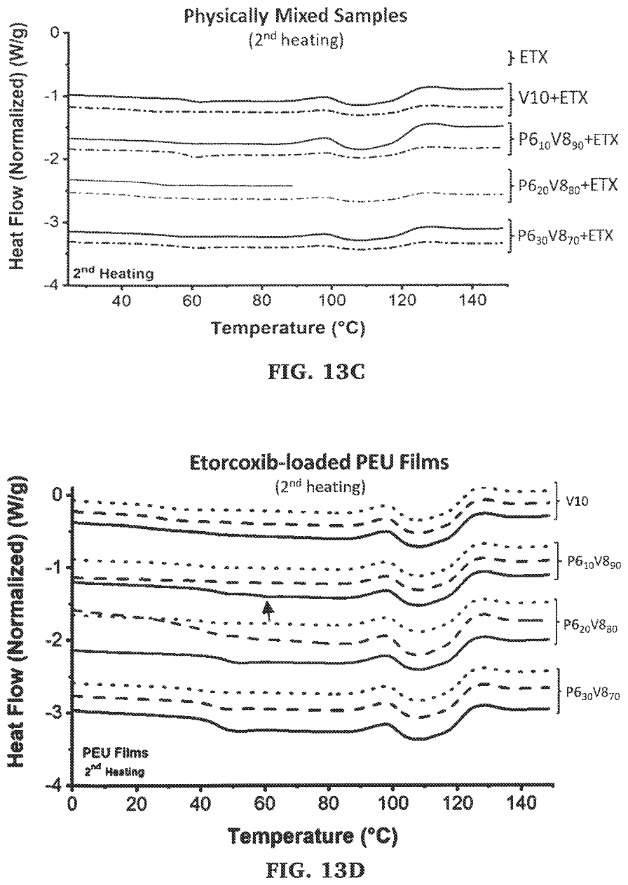

As can be seen, the estimation of etoricoxib content in the film deviates slightly from the actual value. This was attributed to slight drug inhomogeneity in certain areas of the dried film, which were evident visually and by thermo characterization (See FIGS. 13A-D) and to incomplete drug elution with the extraction method. Considering this hurdle, all films that were evaluated for drug release were dissolved following the dissolution to determine the amount of etoricoxib remaining in the film. This value was then adjusted to obtain an accurate cumulative label claim. (See, Tables 2-3, above and Tables 4-6, below).

TABLE 4
Remaining etoricoxib content left in films tested
for polymer composition effect on elution.

| | Amount Left in Film | Amount Left in Film |
|---|---|---|
| Film ID | (mg) | (%) |
| P6₁₀V8₉₀ A | 6.08 | 68.21 |
| P6₁₀V8₉₀ B | 3.88 | 62.47 |
| P6₁₀V8₉₀ c | 3.85 | 59.60 |
| P6₂₀V8₈₀ A | 2.94 | 46.28 |
| P6₂₀V8₈₀ B | 3.03 | 45.10 |
| P6₂₀V8₈₀ C | 2.40 | 43.20 |
| P6₃₀V8₇₀ A | 3.43 | 57.25 |
| P6₃₀V8₇₀ B | 2.52 | 56.10 |
| P6₃₀V8₇₀ C | 2.02 | 46.30 |
| V10 A | 2.13 | 40.22 |
| V10 B | 1.31 | 29.42 |
| V10 C | 0.31 | 11.72 |

TABLE 5
Remaining etoricoxib content left in films
tested for drug-load effect on elution.

| | Amount Left in Film | Amount Left in Film |
|---|---|---|
| Film ID | (mg) | (%) |
| P6₃₀V8₇₀ 20% A | 0.73 | 37.64 |
| P6₃₀V8₇₀ 20% B | 0.77 | 39.78 |
| P6₃₀V8₇₀ 20% C | — | — |
| P6₃₀V8₇₀ 40% A | 4.63 | 72.69 |
| P6₃₀V8₇₀ 40% B | 4.58 | 70.14 |
| P6₃₀V8₇₀ 40% C | — | — |
| V10 20% A | 2.79 | 55.80 |
| V10 20% B | 2.82 | 56.53 |
| V10 20% C | — | — |
| V10 40% A | 6.17 | 67.26 |
| V10 40% B | 4.19 | 57.13 |
| V10 40% C | — | — |

TABLE 6
Remaining etoricoxib content left in films
tested for thickness effect on elution.

| | Amount Left in Film | Amount Left in Film |
|---|---|---|
| Film ID | (mg) | (%) |
| P6₃₀V8₇₀ | 0.39 | 51.31 |
| 78 μm | 0.34 | 47.12 |
| | 0.29 | 46.06 |
| P6₃₀V8₇₀ | 1.74 | 79.76 |
| 200 μm | 1.88 | 89.13 |
| | 1.61 | 76.38 |
| P6₃₀V8₇₀ | 3.89 | 88.96 |
| 430 μm | 4.08 | 90.33 |
| | 4.09 | 92.36 |

In Vitro Etoricoxib Release from Poly(Ester Urea) Films.

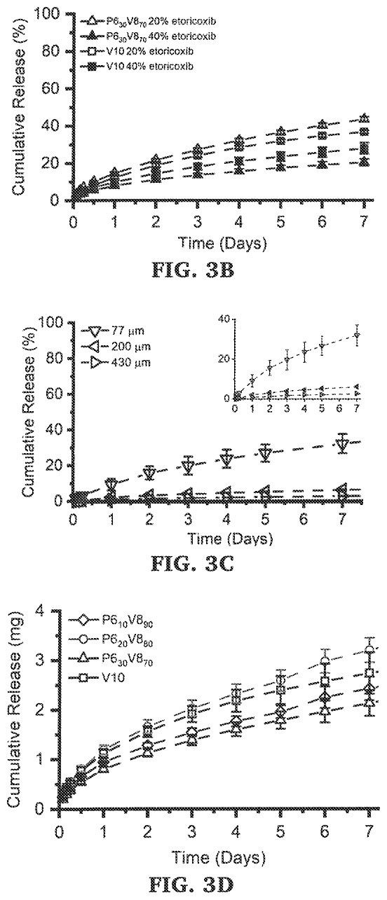

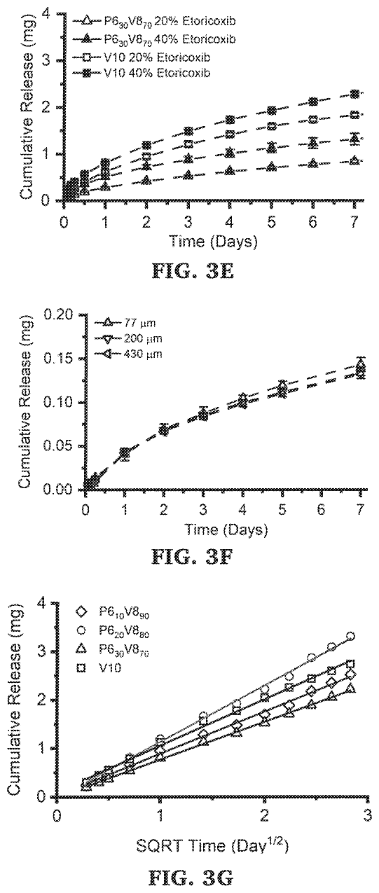

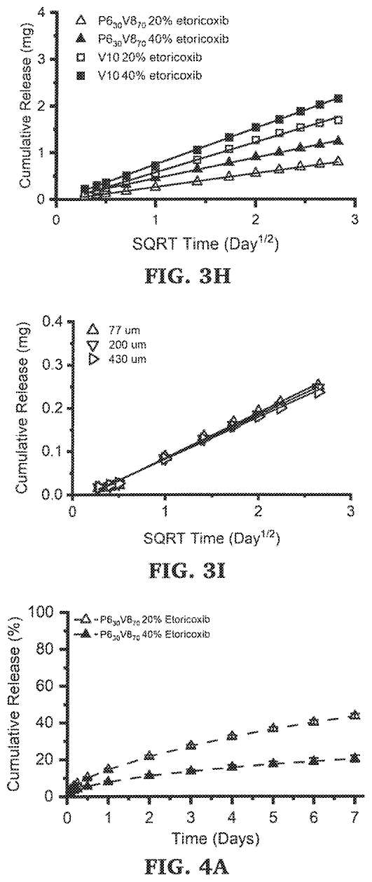

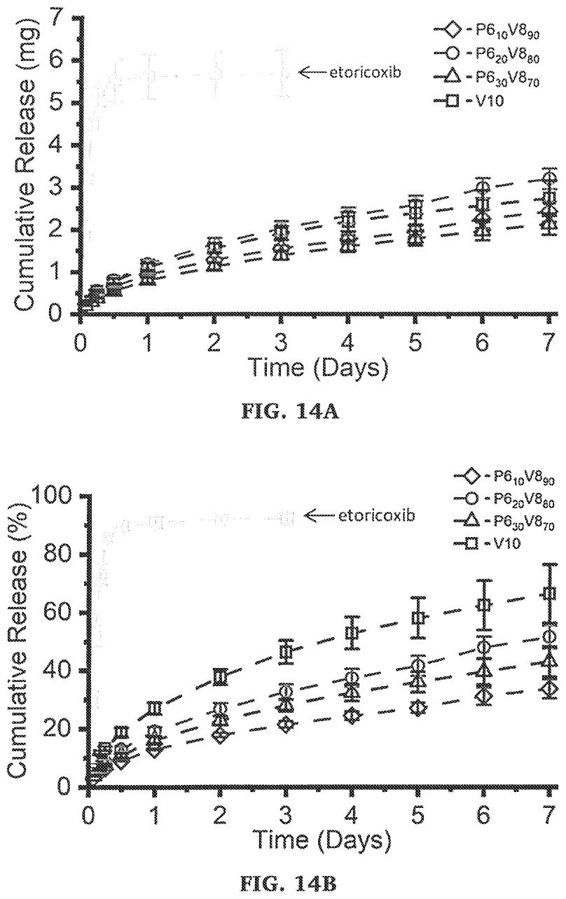

To gain insight on the release behavior of etoricoxib from PEU films, diffusion tests of PEU films according to embodiments of the present invention were was carried out in phosphate-buffered saline (1×PBS) on films prepared using P(1-VAL-10) (V10), 10% 1-PHE-6 P(1-VAL-8) (P6₁₀V8₉₀), 20% 1-PHE-6 P(1-VAL-8) (P6₂₀V8₈₀) and 30% 1-PHE-6 P(1-VAL-8) (P6₃₀V8₇₀) polymers with 20 wt % and 40 wt % loadings of etoricoxib. Overall, the release curves of each polymer and drug loading (FIGS. 3A-I and 4A-B) reveal a sustained release of etoricoxib in comparison to free drug at the same dose. (See FIGS. 14A-B) Although none of the films reach full drug release over the period analyzed, they would be expected to go to completion if left for a longer study. Furthermore, the release profile is nearly constant over time, suggesting it is dictated by one, primary event (e.g. diffusion) and that the polymer degradation plays a limited, if any, role. In contrast, previous literature of poly(lactide-co-glycolide) (PLGA) films has reported curves with multimodal release likely attributed to the swelling and bulk degradation of PLGA and therefore, a nonlinear increase in drug release from the polymer matrix. These results indicate that PEUs could offer a more predictable and controlled release of drug in vitro. Moreover, full release of drug from each film was estimated by fitting the release data to the Higuchi model and extrapolating the linear curve to the respective dose. The primary method of drug diffusion was also estimated using this model. Overall, differences in etoricoxib release varied with the drug loading, the polymer composition, and thickness of the films. These differences will be discussed in turn below.

Overall, the elution profiles (see, FIG. 3A-I) reveal a sustained release of etoricoxib. Although none of the films reach full drug release over the period analyzed, they would be expected to go to completion in a longer study. Furthermore, the release profile maintains continuity over time, suggesting it is dictated by one, primary mechanism (e.g. diffusion). In contrast, previous reports of poly(lactide-co-glycolide) (PLGA) films in the literature have reported elution profiles with multimodal release likely attributed to the bulk degradation of PLGA and therefore, a nonlinear increase in drug release from the polymer matrix. These results indicate that PEUs could offer a more predictable and controllable release of drug in vitro. Full (˜100%) release of drug from each film was estimated by fitting the release data to the Higuchi model the discussion of which will follow. Overall, differences in etoricoxib release varied with drug loading, film polymer composition, and thickness.

### Effect of Drug-Load on Drug Release.

In these experiments, two different drug-loads (20% and 40% (w/w) etoricoxib) were used with 30% 1-PHE-6 P(1-VAL-8) (30 P6V8) and P(1-VAL-10) (V10) films to evaluate differences in diffusion of drug from the polymer matrix. Films were coated at the same speed and gap height in the doctor blade to maintain a consistent thickness (see, Table 7) and film sections were cut into 2 cm squares. Drug release profiles indicate that there are differences when there is a variation of drug-load. Specifically, when weight percent label claim was considered, higher drug-loaded films (40% etoricoxib) were released slower through day 7 in comparison to the 20% drug-load (Table 5). This result correlates well as changes in distribution of drug throughout the film as the amount of drug is increased could be expected. In the higher drug loaded films, etoricoxib is found more within the matrix than the lower drug load, which has etoricoxib primarily on the surface. This difference in distribution proves fruitful for pain management applications as higher homogeneous drug loads will produce a prolonged anesthetic effect by releasing drug for a longer time (i.e. drug in the bulk of the film will take longer to diffuse out). Slower release models could be of great use for pain that is more acute and persistent.

TABLE 7

| | Drug-load | Thickness | Weight of film | Etoricoxib content |
|---|---|---|---|---|
| Polymer | (%) | (μm) | (mg) | (mg) |
| 30% 1-PHE-6 P(1-VAL-8) | 20 | 84 | 21.1 ± 0.6 | 3.5 ± 0.1 |
| 30% 1-PHE-6 P(1-VAL-8) | 40 | 84 | 21.7 ± 2.7 | 6.4 ± 0.2 |
| P(1-VAL-10) | 20 | 85 | 18.2 ± 1.0 | 5.0 ± 0.0 |
| P(1-VAL-10) | 40 | 100 | 25.0 ± 2.9 | 8.3 ± 0.9 |

Characterization of blade-coated 30% 1-PHE-6 P(1-VAL-8) (30 P6V8) and P(1-VAL-10) (V10) blade-coated films for release studies.
Both polymers were prepared with different drug loadings (20% and 40% (w/w) etoricoxib) and etoricoxib content was calculated based on post-dissolution analysis of the film using total content analysis methods and thicknesses were averaged using calipers.
All films were coated at the same gap height on the doctor blade.
Measurements are based on n = 3 films.

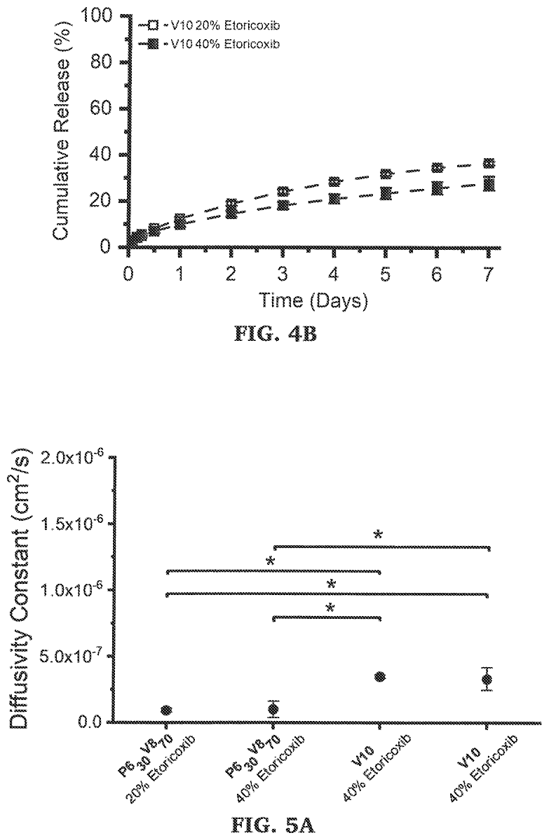

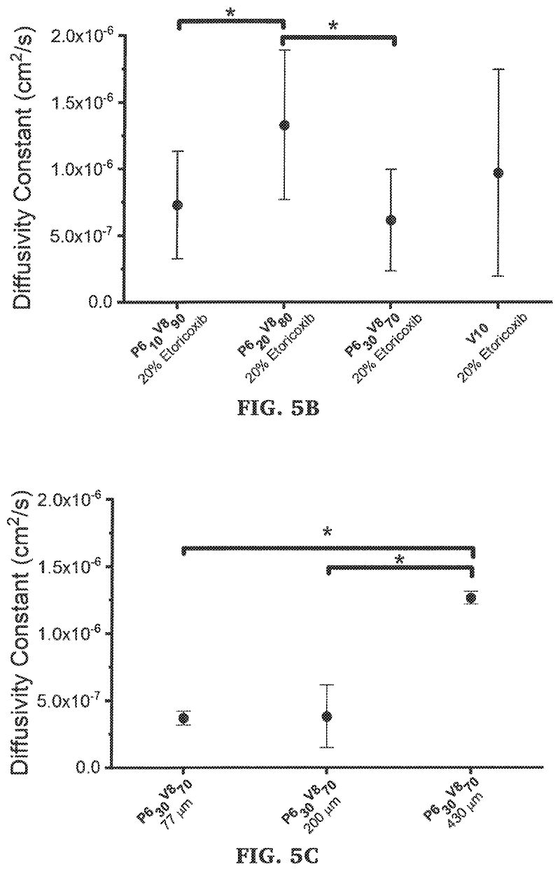

Release curves of etoricoxib-loaded PEU films are shown in FIGS. 4A-B. P6₃₀V8₇₀ (FIG. 4A) and V10 (FIG. 4B) were tested at 20% (open shapes) and 40% (filled shapes) drug-loading. All films were tested in an Agilent 400-DS Dissolution Apparatus 7 at 37° C. with 40 DPM. The cumulative release (%) was calculated according to the remaining amount of etoricoxib in the film after it was retired from the study. Values for total release on day 7 are shown in Table 8, below and in FIGS. 4A-B.

TABLE 8

| | Drug-load | Release at day 7 | Release at day 7 |
|---|---|---|---|
| Polymer | (wt. %) | (mg) | (%) |
| P6₃₀V8₇₀ | 20 | 0.84 ± 0.03 | 43.5 ± 1.5 |
| P6₃₀V8₇₀ | 40 | 1.32 ± 0.12 | 20.4 ± 1.9 |
| V10 | 20 | 1.83 ± 0.04 | 36.6 ± 0.76 |
| V10 | 40 | 2.27 ± 0.04 | 27.6 ± 0.53 |

Total release data of the P(1-VAL-10) (V10) and 30% 1-PHE-6 P(1-VAL-8) (30P6V8) films at day 7 done in triplicate for each data point.
Two drug loads were used to determine the effect on release kinetics.
Values measured are based on n = 3 films.

Distribution of etoricoxib throughout the films can be used to explain the differences in release at day 7. In the 40% films, etoricoxib may be found in a higher concentration in the bulk due to hydrophobic interactions and drug packing, whereas the 20% films have a higher concentration at the surface in comparison, thus releasing more.

### Effect of Polymer Composition on Drug Release.

In these experiments, 10% 1-PHE-6 P(1-VAL-8) (P6₁₀V8₉₀), 20% 1-PHE-6 P(1-VAL-8) (P6₂₀V8₈₀), 30% 1-PHE-6 P(1-VAL-8) (P6₃₀V8₇₀), and P(1-VAL-10) (V10) films were prepared at 20% etoricoxib (w/w) loadings and analyzed for the effect of polymer composition on drug release. The films were coated at the same speed and gap height in the doctor blade (see, Table 3) and sections were cut into 4 cm² squares. Slight variation was shown between films and average thickness. To account for this, the effect of thickness on drug release was also studied. (See Table 9, below). Overall, release of etoricoxib through 7 days was shown to vary with polymer composition as shown by the comparison of the different phenylalanine-valine copolymers (10P6V8, 20P6V8, and 30P6V8) and V10. All films maintained a continuous release profile (see, FIG. 3A). At day 7 of release, the label claim release ranged from 33% to 66% depending on the PEU composition and release rates vary accordingly.

It is believed that the differences observed with polymer composition could be attributed to drug interactions with the polymer chain. Specifically, depending on the two types of amino acids groups analyzed, different intermolecular forces were assumed to be impacting the diffusion of etoricoxib from the polymer matrix. With the phenylalanine-based copolymers, it is believed that the presence of an aromatic group could allow for pi-interactions to occur between the etoricoxib and polymer. These interactions are likely why slower release profiles are observed for the phenylalanine analogues when compared to the V10 homopolymer which lacks the aromatic side chain. Additionally, PEUs have been shown previously to display an elaborate hydrogen bonding network through the urea and carbonyl moieties in the backbone. Although etoricoxib does not have bountiful hydrogen bond donor groups (i.e. an alcohol or amine) compared to other anesthetics, such as lidocaine and bupivacaine, this interaction could help indicate the sustained release profiles for all PEU materials. It is believed that having multiple ways to tune drug release based on chemical composition opens the door for other drug release applications.

TABLE 9

| | Drug-load | Thickness | Weight of film | Etoricoxib content |
|---|---|---|---|---|
| Polymer | (%) | (μm) | (mg) | (mg) |
| 10% 1-PHE-6 P(1-VAL-8) | 20 | 87 | 40.3 ± 8.7 | 7.2 ± 1.5 |
| 20% 1-PHE-6 P(1-VAL-8) | 20 | 51 | 26.6 ± 3.8 | 6.2 ± 0.6 |
| 30% 1-PHE-6 P(1-VAL-8) | 20 | 44 | 27.8 ± 2.5 | 5.0 ± 0.9 |
| P(1-VAL-10) | 20 | 38 | 21.2 ± 5.9 | 4.1 ± 1.4 |

Characterization of blade-coated 10% 1-PHE-6 P(1-VAL-8) (10P6V8), 20% 1-PHE-6 P(1-VAL-8) (20P6V8), 30% 1-PHE-6 P(1-VAL-8) (30P6V8), and P(1-VAL-10) (V10) films for release studies with 20% (w/w) etoricoxib.
Etoricoxib content was calculated based on post-dissolution analysis of the film using total content analysis methods and thicknesses were averaged using calipers.
All films were coated at the same gap height on the doctor blade and measures are based on n = 3 films.

Release curves of etoricoxib-loaded PEU blade coated films are shown in FIG. 3A. 10% 1-PHE-6 P(1-VAL-8) (P6₁₀V8₉₀), 20% 1-PHE-6 P(1-VAL-8) (P6₂₀V8₈₀), 30% 1-PHE-6 P(1-VAL-8) (P6₃V8₇₀), and P(1-VAL-10) (V10) were all tested at 20% drug-load. (See, Table 10). All films were tested in an Agilent 400-DS Dissolution Apparatus 7 at 37° C. with 40 DPM. Cumulative release (%) was calculated according to the remaining amount of etoricoxib in the film after it was retired from the study.

TABLE 10

| | Drug-load | Release at day 7 | Release at day 7 |
|---|---|---|---|
| Polymer | (wt. %) | (mg) | (%) |
| P6₁₀V8₉₀ | 20 | 2.42 ± 0.24 | 33.7 ± 3.3 |
| P6₂₀V8₈₀ | 20 | 3.20 ± 0.25 | 51.5 ± 4.0 |
| P6₃₀V8₇₀ | 20 | 2.13 ± 0.26 | 43.0 ± 5.2 |
| V10 | 20 | 2.74 ± 0.42 | 66.3 ± 10.1 |

Total release data of 20% etoricoxib (w/w) films at day 8 done in triplicate for each data point.
Values measured are based on n = 3 films.

### Effect of Film Thickness on Drug Release.

By varying the concentration of the drug-loaded solution and the gap height of the doctor blade, different film thicknesses were controlled. Altering the thickness of the film changes the distributing of drug within the matrix and, therefore, the ability of the drug to diffuse out of the film. The polymer used for this study was the 30P6V8 copolymer and the etoricoxib load was kept constant at 20%. Thicknesses obtained were 77, 200, and 430 μm. 8 mm circular punches were cut out of the large strip and films were placed in phosphate buffer (37° C., agitating at 80 rpm) solution to test for release. The full dimensions of these films can be found in the SI. The release curves indicate that the thicker the film, the longer the duration of anesthesia, or the longer the time for drug release. Explicitly, these results show that thicker films are able to pack more drug in the shape of the film homogeneously, therefore giving rise to a film that will release etoricoxib at the same rate, but a longer duration. Moreover, as all of these films release the same amount of drug per time point (Table 11), it is fair to say that these films are releasing drug at the same rate (etoricoxib is diffusing out of the film at the same rate) and that etoricoxib is homogeneously distributed.

TABLE 11

| | | | | Etoricoxib |
|---|---|---|---|---|
| | Drug-load | Thickness | Weight of film | Content |
| Polymer | (%) | (μm) | (mg) | (mg) |
| P6₃₀V8₇₀ | 20 | 77 ± 8.7 | 2.1 ± 0.5 | 0.5 ± 0.1 |
| P6₃₀V8₇₀ | 20 | 200 ± 8.0 | 11.4 ± 0.3 | 2.2 ± 0.1 |
| P6₃₀V8₇₀ | 20 | 430 ± 17.3 | 25.7 ± 1.4 | 5.0 ± 0.1 |

Characterization of blade-coated 30% 1-PHE-6 P(1-VAL-8) (P6₃₀V8₇₀) films with various thickness with 20% (w/w) etoricoxib.
Etoricoxib content was calculated based on post-dissolution analysis of the film using total content analysis methods and thicknesses were averaged using calipers (n = 3).
Films were coated on different heights on the blade coater to obtain different thicknesses.
Each measurement is based on n = 3.

Cumulative release curves of P6₃₀V8₇₀ films with 20% etoricoxib loading at various thicknesses (77, 200, and 430 μm) are shown in FIG. 3C. The films were placed in phosphate buffer solution and kept at 37° C. agitating at 80 rpm. (See, Table 12) Cumulative release (%) was calculated according to the remaining amount of etoricoxib in the film after it was retired from the study.

TABLE 12

| Thickness | Drug-load | Release at day 7 | Release at day 7 |
|---|---|---|---|
| (μm) | (wt. %) | (mg) | (%) |
| 77 | 20 | 0.14 ± 0.008 | 32.0 ± 5.3 |
| 200 | 20 | 0.13 ± 0.003 | 6.1 ± 0.3 |
| 430 | 20 | 0.13 ± 0.004 | 2.6 ± 0.1 |

Total release data of 30% 1-PHE-6 P(1-VAL-8) (P6₃₀V8₇₀) 20% etoricoxib (w/w) films with different thicknesses.
Values measured are based on n = 3 films.

As can be seen in Table 13, below, the etoricoxib amount in film punches taken for analysis of average drug content. The theoretical (predicted based on the weight of the film and the drug loading) values were compared to the actual amount of etoricoxib calculated according to HPLC analysis. (See, Table 13) The accuracy of the theoretical predicted value was determined by obtaining a percentage of the theoretical drug load versus the actual drug load. Overestimations were attributed to aggregation of drug in the specific area that was cut out. Samples (n=3) were tested following dissolution and the cumulative etoricoxib amount was summed to the amount extracted from the film upon completion of the study. (Table 13).

TABLE 13

| | Drug Load | Weight of Film | Theoretical Drug | Actual Drug | Theoretical |
|---|---|---|---|---|---|
| Thickness | (wt. %) | (mg) | Weight (mg) | Weight (mg) | Accuracy (%) |
| 77 μm | 20 | 2.1 ± 0.5 | 0.4 ± 0.1 | 0.5 ± 0.1 | 90.9 ± 16.1 |
| 200 μm | 20 | 11.4 ± 0.3 | 2.3 ± 0.1 | 2.1 ± 0.1 | 103.9 ± 4.5 |
| 430 μm | 20 | 25.7 ± 1.4 | 5.1 ± 0.3 | 5.0 ± 0.1 | 103.2 ± 4.4 |

Higuchi Model Fitting for Etoricoxib Release from PEU Films.

Release results were fit to a kinetic model to quantitatively understand the mechanism behind which etoricoxib is releasing from the films and to predict how long it would take for full release of drug. The Higuchi model was determined to best fit the results based on the criteria that the model accounts for. The model assumes (i) total sink condition at all times, (ii) release from only one-dimension, (iii) initial drug concentration in the film is much higher than the drug solubility, (iv) drug particles are much smaller than the film thickness, (v) swelling and dissolution of the film are negligible, and (vi) the diffusivity of the drug is constant. See, e.g., T. Higuchi, Mechanism of sustained-action medication. Theoretical analysis of rate of release of solid drugs dispersed in solid matrices, Journal of Pharmaceutical Sciences, 52 (1963) 1145-1149, the disclosure of which is incorporated herein by reference in its entirety. As the release matrices in this case were thin films, these assumptions were considered to be maintained, and therefore the model was deemed acceptable to use. To fit the data, the cumulative release (mg) was plotted against the square root of time, as described (FIG. 3C). All plots remained linear for the entire time frame of data collection (R²=0.99). From these plots, the diffusivity constant was determined using the following equation:

D=(slope/A)²/2CCₛ
Where the slope is the amount of drug released at time t divided by the square root of time, A is the area of the film, C is the initial drug concentration (or the drug-load in the polymer), C_{S} is the drug solubility in the media (0.5 mg/mL) and D is the diffusivity constant. This constant not only indicates the mobility of the drug from the film into the media, but also can be used to calculate for the time of total release using the maximum amount of drug within the film (Table 11).

Higuchi model fitting for all tested films is shown in FIGS. 3G-I. (See also, Table 11, below.) The respective polymers are labeled with different colors and the 20% drug-loaded films are labelled with empty shapes while the 40% films are filled shapes. Variables analyzed for diffusivity were drug-load (FIG. 3H), polymer composition (FIG. 3G), and thickness (FIG. 3I). Diffusivity constants were calculated using the linear fit equation and used to estimate the time for 100% of the drug to be released. All linear fits had a Pearson square value of 0.97 or above.

TABLE 14
Higuchi model data of all factors analyzed.

| | | Drug-load | Thickness | Diffusivity Constant | t_{100%} |
|---|---|---|---|---|---|
| Group | Polymer | (%) | (μm) | (cm²/s) | (days) |
| A | P6₃₀V8₇₀ | 20 | 84 | 9.35E−08 ± 5.49E−09 | 37 ± 2 |
| | P6₃₀V8₇₀ | 40 | 83.5 | 9.93E−08 ± 2.46E−08 | 191 ± 46 |
| | V10 | 20 | 85 | 3.49E−07 ± 5.58E−09 | 63 ± 1 |
| | V10 | 40 | 100 | 3.30E−07 ± 3.46E−08 | 93 ± 26 |
| B | P6₁₀V8₉₀ | 20 | 87 | 7.29E−07 ± 1.62E−07 | 64 ± 15 |
| | P6₂₀V8₈₀ | 20 | 51 | 1.33E−06 ± 2.26E−07 | 27 ± 2 |
| | P6₃₀V8₇₀ | 20 | 44 | 6.14E−07 ± 1.53E−07 | 37 ± 9 |
| | V10 | 20 | 38 | 9.69E−07 ± 3.12E−07 | 15 ± 6 |
| C | P6₃₀V8₇₀ | 20 | 78 | 3.69E−07 ± 2.17E−08 | 53 ± 22 |
| | P6₃₀V8₇₀ | 20 | 200 | 3.81E−07 ± 9.43E−08 | 1610 ± 61 |
| | P6₃₀V8₇₀ | 20 | 430 | 1.27E−06 ± 1.93E−08 | 8641 ± 1750 |

Variables analyzed for diffusivity were (A) drug-load (see, FIG. 3G), (B) polymer composition (see, FIG. 3G), and film thickness (see FIG. 3I).
Diffusivity constants were calculated using the linear fit equation and used to estimate the time for 100% of the drug to be released.
All linear fits had a Pearson square correlation coefficient of 0.97 or above.
Statistical significance was determined by a one-way ANOVA with Tukey post hoc analysis of the diffusivity constants of each film.
A value of p < 0.05 was considered significant.
Mass data is reported as means ± standard deviation.

Differences in total release time can be noted for polymer composition, drug-load, and thickness with the most etoricoxib releasing from 20P6V8 and V10 at 20% drug-load, relative to all time points measured. This is more accurately suggested by the diffusivity constants, which can be compared relatively. The constants vary between all tested factors, suggesting that they impact how the etoricoxib is distributed in the film and therefore how it releases from the films. Total amount released, diffusivity constant, and the predicted time for total release all follow the same trend between all films tested; any of the three can be accurately used to compare differences in release between samples. These results quantitatively correlate with the release results discussed above. Specifically, the higher drug loaded and thicker films were predicted to take longer to release 100% of the etoricoxib from the film, administering longer analgesia to the patient. Additionally, the diffusivity constants for the various polymer compositions describe the movement of etoricoxib out of the film in a similar manner that was described above. The phenylalanine copolymer diffusivity constants suggest a delayed release of etoricoxib that can be explained by the effects of intermolecular forces (e.g. pi-pi interactions). Moving forward, the Higuchi model can be used to accurately tune the matrix in a way to obtain a desired release profile and end point for dosage-specific applications.

### In Vivo Release Results.

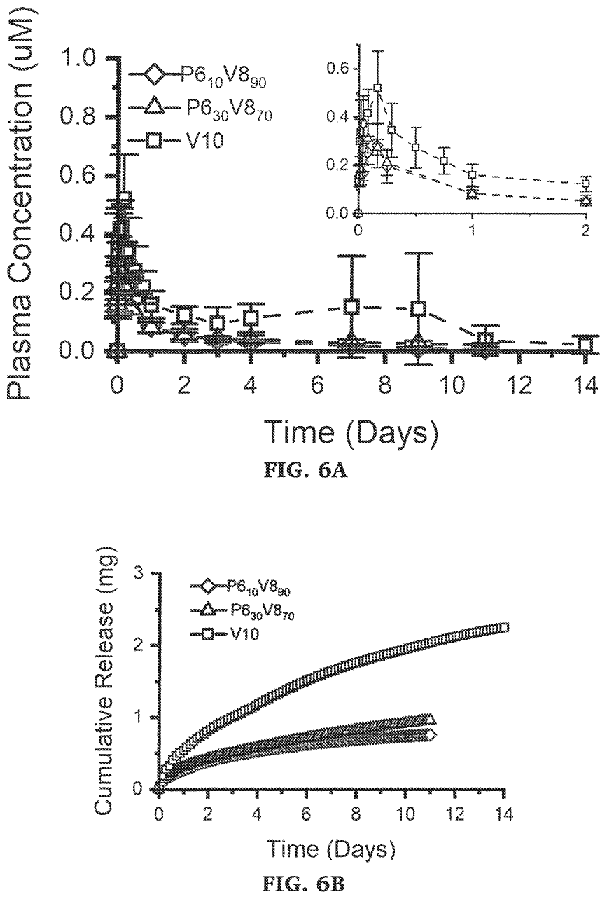

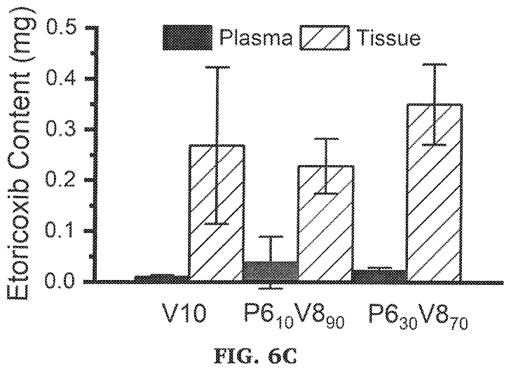

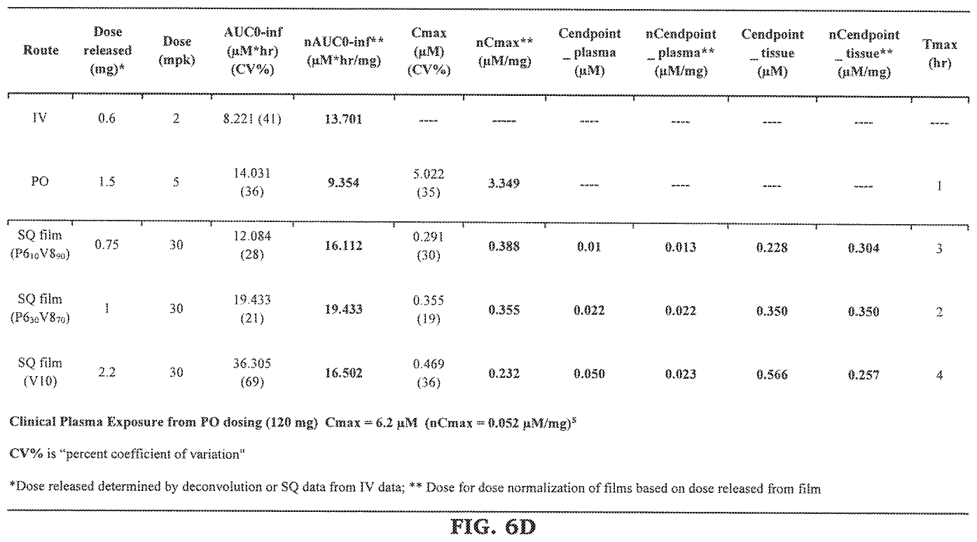

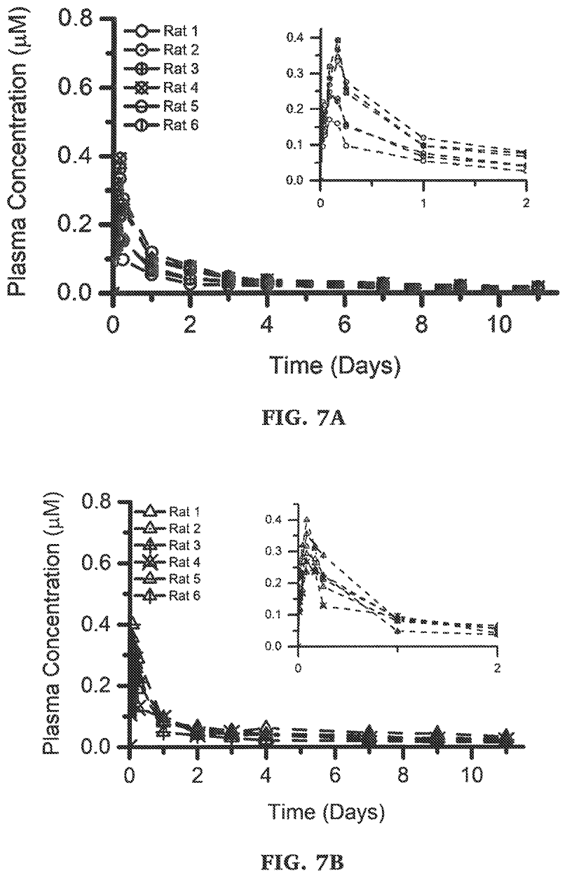

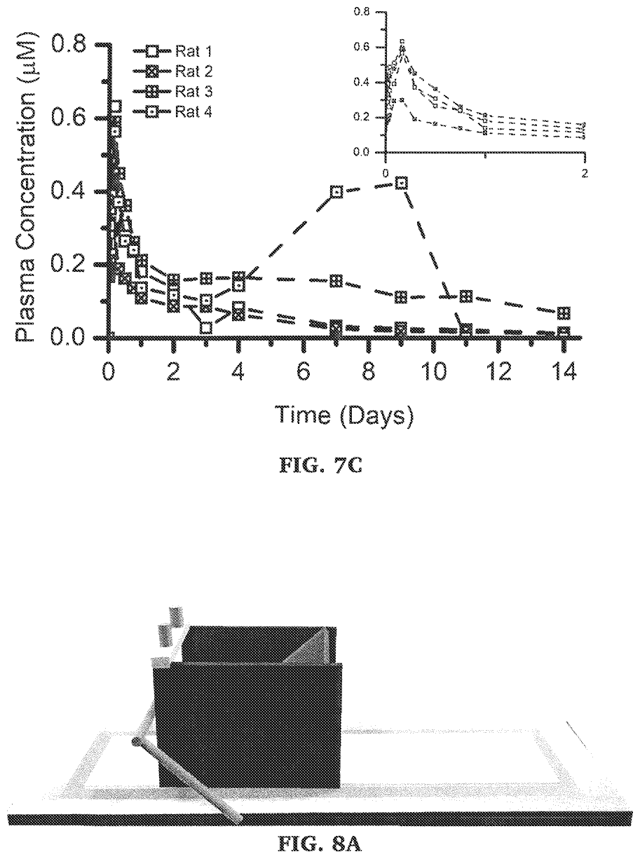

Comparing the overall data from the copolymer and V10 films, the V10 etoricoxib films show faster release of etoricoxib via both in vitro release data (FIG. 3A) and in vivo plasma exposure (FIGS. 6A-D). The etoricoxib films show an initial “burst” of etoricoxib exposure followed by a rapid decline in exposure out to 48 hr, when close to steady state is reached. Variability between animals is not significant with CV % of 20-30% for both AUC and Cₘₐₓ values for most etoricoxib films (Figure FIGS. 7A-C). The outlier being the V10 etoricoxib films which had 1 animal that deviated from steady state showing a spike in concentration starting at 168 h and continuing until 216 h. This animal had higher exposure then the other 2 animals, causing the higher CV % (69%) in AUC calculations for the V10 films. However, given the limited number of subjects it is hard to tease out the real significance of these values.

The comparison between subcutaneous (SQ) and oral (PO) administration for etoricoxib appears to show systemic dose normalized etoricoxib exposure that is higher in rats with delivery from implanted films over PO dosing (FIGS. 6A-D). However, this current comparison does not account for potential differences in individual animal exposure and the limited number of subjects. Importantly, etoricoxib plasma dose normalized Cₘₐₓ concentration is ˜10 folder lower with delivery from implant compared to PO administration. At the study endpoints, etoricoxib concentration from SQ implanted films is 7-23 fold higher in local tissue then plasma in rats. The lower Cₘₐₓ and higher local tissue exposure could be beneficial with respect to lower dose needed and hence lower systemic adverse events. For comparison, the dose normalized amount of etoricoxib in local rat tissue is 3.5-12 fold higher than dose normalized Cₘₐₓ from plasma in a clinical study with PO dosed etoricoxib.

Deconvolution of in vivo rat data showed the mean cumulative input into the system which generally estimates release of drug from the films. As expected, the etoricoxb films show differences in mean cumulative input with the V10>P6₂₀V8₈₀/P6₃₀V8₇₀ (FIGS. 6A-D). This suggests faster release from V10 films which is in line with in vitro data, though the in vivo data exhibits slower etoricoxib elution in comparison to the PEU films ran in vitro, which is likely due to the increased film thickness of implanted films. Calculations based on weight difference of the films pre-implant and following the 11-day study showed that a large majority of etoricoxib remained in the excised film after study completion. Unfortunately, due to issues with drying the excised film a quantitative assessment of the etoricoxib remaining in the films was not applicable, but the deconvolution data yields a relative measure of how much is left in the film at the study conclusion. More extensive analysis of the excised tissue was undertaken and showed an average of only ˜0.02% of the expected etoricoxib dose being recovered in the removed tissue. Overall, the data suggests the in vivo release of etoricoxib from these films will occur beyond the duration tested.

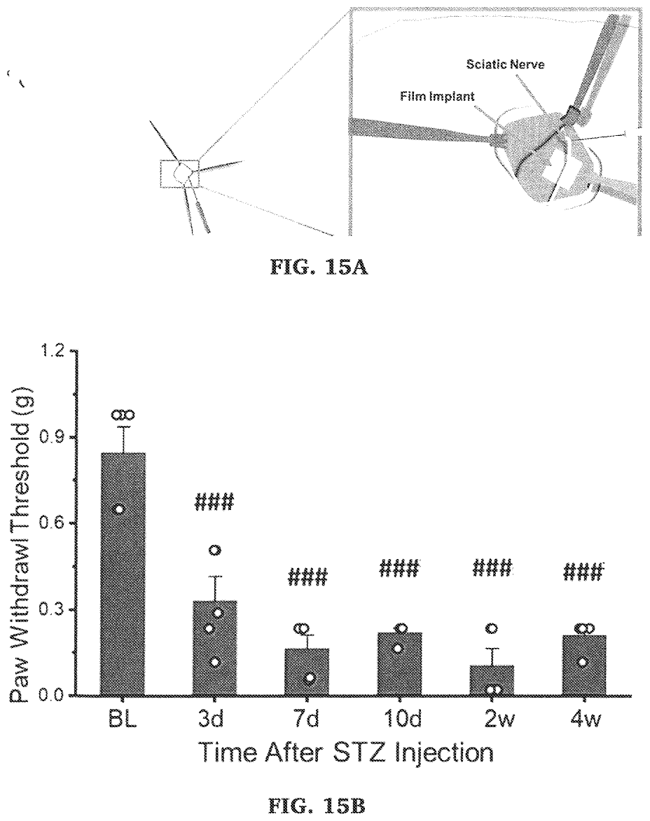

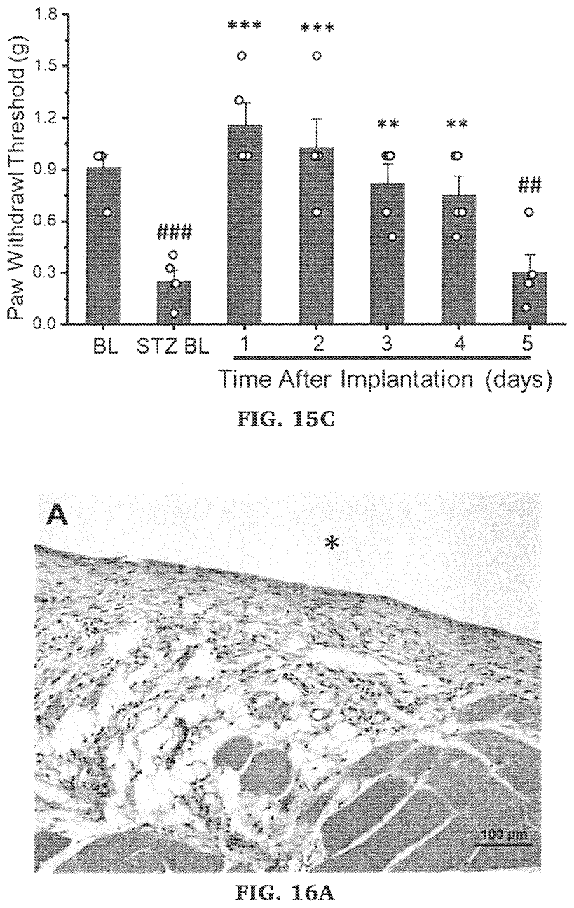

Film Efficacy in pain inhibition. To demonstrate efficacy of pain relief for drug loaded PEU films we wrapped the sciatic nerve with films loaded with etoricoxib into mice with neuropathic pain in a well-defined diabetic neuropathy model. (See FIG. 15A) Neuropathic pain was induced by intraperitoneal injection of streptozotocin (STZ) (75 mg/kg). Baseline paw withdraw threshold was measured with Von Frey filaments before and after induction of neuropathic pain with STZ injection. PEU films containing etoricoxib were placed around the ipsilateral sciatic nerve and paw withdrawal threshold was measured daily for 5 days following PEU insertion (FIG. 15B-C). At baseline (BL) FIG. 15B-C show the onset of mechanical allodynia as a marked decrease in paw withdraw threshold for control STZ treated mice without local analgesic (STZ-BL) compared to before treatment (BL). Etoricoxib loaded PEU film (40% w/w, ˜0.6 mg API, 40 m thick) showed significant reduction of mechanical allodynia as indicated by improvements in paw withdrawal threshold beyond pre-treatment baseline for the first 2 days, with a continued reduction of allodynia through day 4 (P<0.001). This data also implies sustained pain relief of the locally implanted film via peri-sciatic nerve block, as the pain signal in a hind paw is conducted to the brain through the sciatic nerve. Therefore, local release of analgesic is sufficient to relieve pain that may develop at a further distance but originates at the surgical site. Considering analgesia is crucial for the first 3-5 days following a procedure, these results demonstrate the ability of PEU films of the present invention to act as an effective solution for local delivery of analgesic post-operatively. While the anti-inflammatory ability of these films was not directly monitored, it is expected that these films could also combat inflammation caused locally to the site of surgery or be used for arthritic pain considering the inherent nature of etoricoxib as a selective COX-2 inhibitor.

Histology and Inflammatory Response. Subcutaneous thigh implants were performed to assess the inflammatory response induced by etoricoxib-loaded PEU films. (See, FIG. 15A) No evidence of analgesia via a Von Frey measurements was noted with subcutaneous implantation in the hind thigh. (See, FIGS. 15B-C). This was attributed to the localized delivery of drug and inability of the film to provide nerve block systemically at a distance from the nerve. Little to no evidence of inflammation or non-healing wounds in the muscle was noted at day 14 in serial cuts of the tissue samples.

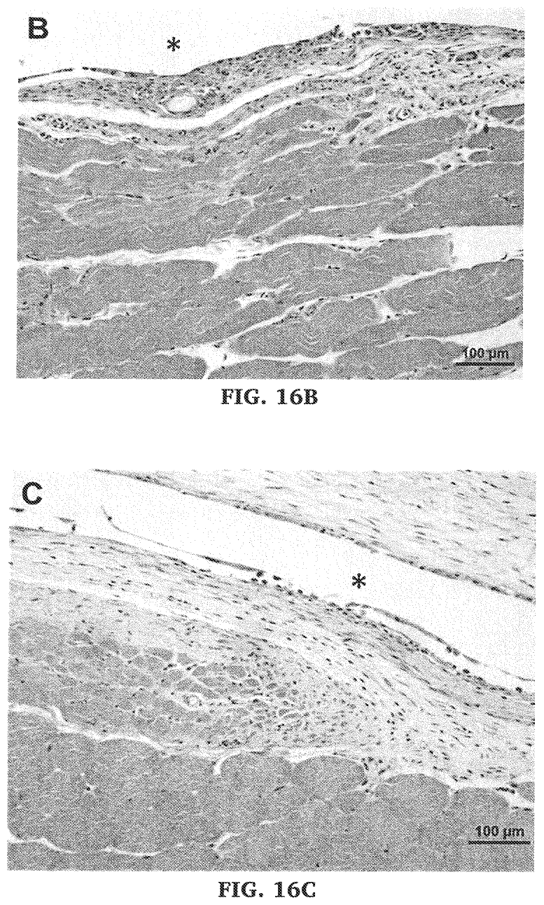

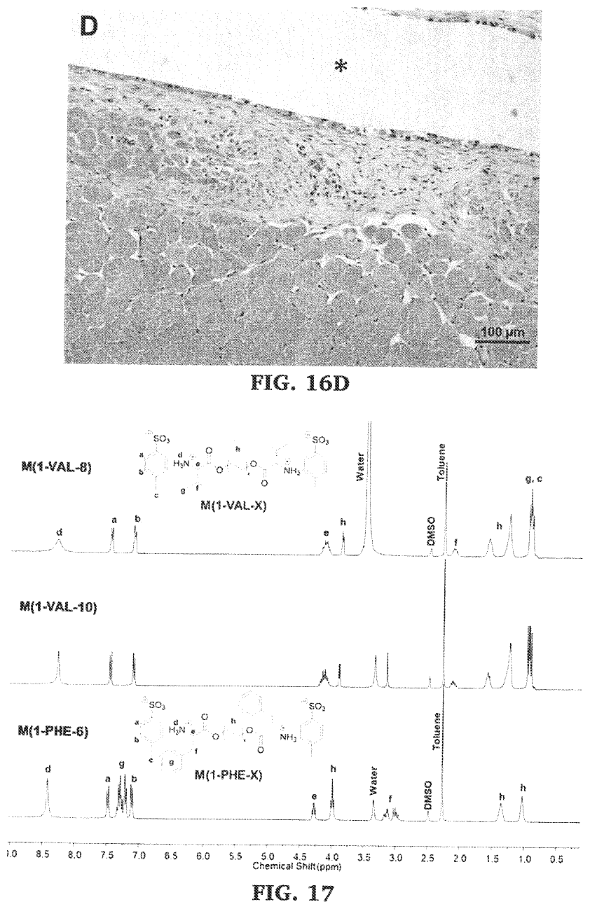

No significant histopathological differences were noted in the tissue reactions to blank or ETX-loaded PEU films (FIGS. 16A-D). The subcutaneous tissue pocket surrounding the implanted films was characterized by a thin circumscribing capsule of macrophages, fibroblasts and lymphocytes with occasional polymorphonuclear leukocytes (neutrophils) and rare foreign-body giant cells. There was no evidence that ETX-loaded films reduced the quantity of inflammatory cells, neovascularization or fibrosis. The experimental design and time course did not allow an assessment of the effect of ETX on wound healing, but there was no indication of differences in the cellular composition of the healing process or reaction to the overlying PEU films at the 14-day time point.

Postoperative pain management remains a challenge following major surgery. With the over prescription of opioid pain management drugs, alternative strategies to manage pain must be employed. Herein, a series of poly(ester urea) homopolymers and copolymers were synthesized, mixed with etoricoxib, and fabricated in to local drug delivery films. The recommended dose of etoricoxib is anywhere from 30-120 mg daily, depending on the patient and the severity of pain. By changing various factors of fabrication for PEU films according to the present invention, the tunable, controlled release of a model analgesic compound has been achieved. On the basis of film dimensions (area and thickness), drug load, and/or polymer composition, the diffusion of drug out of the matrix can be selectively controlled to achieve the appropriate amount of analgesia needed. This data is used in tandem with Higuchi modeling to identify a film specific to the intended dose to release drug over a specific period of time. Using the Higuchi model, total release of etoricoxib from the films was estimated at 21-187 days, depending on the polymer's chemical composition and/or drug-load. In a rat model, the films produced an apparent numbing sensation, suggesting effective and efficacious delivery of the analgesic compound, proving the success of these systems as a local delivery device. Further local application of etoricoxib-loaded film effectively relieved neuropathic pain for >4 days in a mouse model while producing limited inflammatory response in local tissue.

### EXAMPLES

The following examples are offered to more fully illustrate the invention, but are not to be construed as limiting the scope thereof. Further, while some of examples may include conclusions about the way the invention may function, the inventor do not intend to be bound by those conclusions, but put them forth only as possible explanations. Moreover, unless noted by use of past tense, presentation of an example does not imply that an experiment or procedure was, or was not, conducted, or that results were, or were not actually obtained. Efforts have been made to ensure accuracy with respect to numbers used (e.g., amounts, temperature), but some experimental errors and deviations may be present. Unless indicated otherwise, parts are parts by weight, molecular weight is weight average molecular weight, temperature is in degrees Centigrade, and pressure is at or near atmospheric.

### Materials

1,10-decanediol, 1,8-octanediol, 1,6-hexanediol, sodium carbonate, p-toluenesulfonic acid monohydrate, and triphosgene were purchased from Sigma Aldrich (Milwaukee, WI). Toluene, chloroform, acetone, and N,N-dimethylformamide were purchased from Fischer Scientific (Pittsburgh, PA). L-valine and L-phenylalanine were purchased from Acros (Pittsburgh, PA). HEPES buffer and collagenase, type II were purchased from MP Biomedicals, LLC (Solon, OH). Etoricoxib was provided by Merck & Co. Inc. (Rahway, NJ). All solvents were reagent grade and all chemicals were used without further purification unless otherwise stated.

### Characterization.

Unless indicated otherwise, the following methods and equipment were used in the analysis set forth below. Proton (¹H) nuclear magnetic resonance (NMR) spectra were obtained using a 300 MHz Varian NMR spectrometer. Chemical shifts are reported in ppm (δ) and referenced to residual solvent resonances (¹H NMR DMSO-d₆ 2.50 ppm). Multiplicities were explained using the following abbreviations: s=singlet, d=doublet, t=triplet, br=broad singlet, and m=multiplet. Size exclusion chromatography (SEC) was performed using an EcoSEC HLC-8320GPC (Tosoh Bioscience, LLC) equipped with a TSKgel GMHHR-M 7.8 mm I.D.×30 cm mixed bed column and a refractive index (RI) detector.

The number average molecular mass (Mₙ), weight average molecular mass (M_{w}), and molecular mass distribution (Ð_{M}) for each sample was calculated against a calibration curve of poly(styrene) standards (PStQuick MP-M standards, Tosoh Bioscience LLC) with DMF as the eluent (1.0 mL/min at 50° C.) with respect to Examples 1-8 and THF as the eluent (0.35 mL/min at 40) with respect to Examples 9-10. Differential scanning calorimetry (DSC) was performed using a TA Q200 with heating and cooling cycles (20° C./min) and temperature sweeps from 0 to 150° C. for Examples 1-8 and using a TA Discovery DSC 250 with heating and cooling cycles (10° C./min) with temperature sweeps from 0 to 100° C. for Examples 9-10. The glass transition temperature (T_{g}) was determined from the midpoint of the second heating cycle curve. Thermogravimetric analysis (TGA) was performed using a TA Q500 with heating ramps of 20° C./min in the temperature range from 0 to 500° C./min for Examples 1-8 and using a TA Discovery TGA 550 with heating ramps of 10° C./min in the temperature range from 0 to 500° C. for Examples 9-10. The degradation temperature (T_{d}) was determined from 10% mass loss. Eluted etoricoxib solutions were run in an Agilent 1290 Infinity high-pressure liquid chromatograph System (HPLC) equipped with an Ascentis® Express C18 column (5×0.3 cm, 2.7 m) and a 0.1% phosphoric acid:acetonitrile (77:23) mobile phase. The etoricoxib concentration was determined by reference to external standards using Empower Software (Waters).

### Example 1

Synthesis of Di-p-toluenesulfonic Acid Monomer Salts

Synthesis of di-p-toluenesulfonic acid monomer salts were carried out according to methods described previously. See, e.g., J. Yu, F. Lin, P. Lin, Y. Gao, M. L. Becker, Phenylalanine-Based Poly(ester urea): Synthesis, Characterization, and in vitro Degradation, Macromolecules, 47 (2014) 121-129, the disclosure of which is incorporated herein by reference in its entirety. Briefly, in a 2 L round bottom flask, diol (1 eq.), L-amino acid (2.25 eq.), p-toluenesulfonic acid monohydrate (2.32 eq.), and toluene (1000 mL) were added and equipped with a stir bar. A Dean-Stark trap attached with a condenser was fastened to the round bottom flask and the reaction was heated to 110° C. and refluxed for 24 h. The reaction was cooled to room temperature, and the resulting white precipitate was isolated by vacuum filtration in a Buchner funnel. The product was dissolved in boiling water (2 L), hot vacuum filtered, and cooled to room temperature. This process was repeated three times to ensure product purity (FIG. 17).

### Example 2

Synthesis of Di-p-toluenesulfonic Acid Salts of Bis(L-valine)-Octane 1,8-Diester Monomer (1-VAL-8).

Synthesis of di-p-toluenesulfonic acid salts of bis(L-valine)-octane 1,8-diester (1-VAL-8) (V8) was carried out according to previously published procedures. See, e.g., J. Yu, F. Lin, P. Lin, Y. Gao, M. L. Becker, Phenylalanine-Based Poly(ester urea): Synthesis, Characterization, and in vitro Degradation, Macromolecules, 47 (2014) 121-129, the disclosure of which is incorporated herein by reference in its entirety. Briefly, in a 2 L round bottom flask, 1,8-octanediol (40 g, 0.28 mol, 1 eq.), L-valine (74 g, 0.63 mol, 2.25 eq.), p-toluenesulfonic acid monohydrate (126.00 g, 0.65 mol, 2.32 eq.), and toluene (1000 mL) were added and equipped with a stir bar. A Dean-Stark trap attached with a condenser was fastened to the round bottom flask and the reaction was heated to 110° C. and refluxed for 24 h. The reaction was cooled to room temperature, and the resulting white precipitate was isolated by vacuum filtration in a Buchner funnel. The product was dissolved in boiling water (2 L), hot vacuum filtered, and cooled to room temperature. This process was repeated three times to ensure product purity. ¹H NMR (300 MHz, 303 K, DMSO-d₆): δ=0.91-0.97 (m, 12H), 1.28 (s, 8H), 1.59 (m, 4H), 2.10-2.21 (m, 2H), 2.27 (s, 6H), 2.50 (m, DMSO), 3.83-3.84 (d, ³J_{H-H}=4.4 Hz, 2H), 4.05-4.15 (m, 4H), 7.07-7.09 (d, ³J_{H-H}=7.9 Hz, 4H, aromatic H), 7.43-7.46 (d, ³J_{H-H}=9.9 Hz, 4H, aromatic H), 8.27 (br, 6H) ppm.

### Example 3

Synthesis of Di-p-toluenesulfonic Acid Salts of Bis(L-Valine)-Decane 1,10-Diester Monomer. (1-VAL-10).

Synthesis of the di-p-toluene sulfonic acid of bis(L-valine)-decane 1,10-diester (1-VAL-10) (V10) was carried out using the method described above, but using 1,10-decanediol. Briefly, 1,10-decanediol (40.00 g, 0.23 mol, 1 eq.), L-valine (62.09.83 g, 0.53 mol, 2.25 eq.), p-toluenesulfonic acid monohydrate (104.81 g, 0.55 mol, 2.32 eq.), and toluene (1000 mL) were added and equipped with a stir bar. ¹H NMR (300 MHz, 303 K, DMSO-d₆): δ=0.93-0.99 (m, 12H), 1.26 (s, 8H), 1.58-1.62 (m, 4H), 2.09-2.17 (m, 2H), 2.29 (s, 6H), 2.50 (m, DMSO), 3.17-3.36 (s, H₂O), 3.91-3.93 (d, ³J_{H-H}=4.5 Hz, 2H), 4.08-4.24 (m, 4H), 7.11-7.13 (d, ³J_{H-H}=7.9 Hz 4H, aromatic H), 7.44-7.47 (d, ³J_{H-H}=7.6 Hz4H, aromatic H), 8.29 (br, 6H) ppm.

### Example 4

Synthesis of Di-p-toluenesulfonic Acid Salts of Bis(L-phenylalanine)-Hexane 1,6-Diester Monomer. (1-PHE-6).

Synthesis of di-p-toluene sulfonic acid of bis(L-phenylalanine)-hexane 1,6-diester (1-PHE-6) was carried out using the method described in Example 1 above, but using 1,6-hexanediol (40.00 g, 0.34 mol, 1 eq.), L-phenylalanine (128.79 g, 0.78 mol, 2.25 eq.) and p-toluene sulfonic acid (154.74 g, 0.81 mol, 2.4 eq.)..¹H NMR (300 MHz, 303 K, DMSO-d₆): δ=1.05 (s, 4H), 1.38 (m, 4H), 2.29 (s, 6H), 2.50 (m, DMSO), 2.97-3.20 (m, 4H), 3.36 (s, H₂O), 3.99-4.03 (t, ³J_{H-H}=6.4 Hz, 4H), 4.27-4.31 (m, 2H), 7.11-7.14 (d, ³J_{H-H}=7.9 Hz, 4H), 7.21-7.35 (m, 10H), 7.48-7.51 (d, ³J_{H-H}=7.9 Hz, 4H), 8.44 (s, 6H) ppm.

### Example 5

Synthesis of Counterion-protected L-phenylalanine based diester monomer

Counterion protected L-phenylalanine based diester monomer (1-PHE-6) was synthesized using the route shown in Scheme 4, below, and reported in Stakleff, K. S.; Lin, F.; Smith Callahan, L. A.; Wade, M. B.; Esterle, A.; Miller, J.; Graham, M.; Becker, M. L. Acta Biomaterialia 2013, 9, 5132, the disclosure of which is encorporated herein by reference in its entirety. See also, U.S. Pat. Nos. 9,745,414, 9,988,492, 10,280,261, 10,414,864, 10,537,660, US Published Application No. 2019/0167838 and 2020/368164, International Patent Publication Nos. WO 2020/226622 and WO 2020/226646, the disclosures of which are also encorporated herein by reference in their entireties.

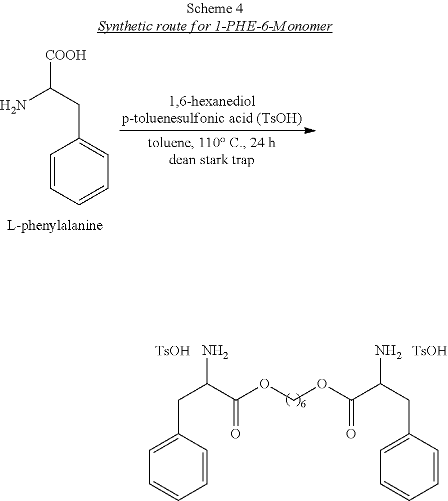 [SMILES: NC(Cc1ccccc1)C(=O)O ; "TsOH" ; NC(Cc1ccccc1)C(=O)OCOC(=O)C(N)Cc1ccccc1 ; "TsOH"]

Scheme 4 shows the condensation of the L phenylalanine with the diol (in this case 1,6 hexane diol) to form the acid protected 1-PHE-6 monomer. The TsOH acidifies the solution conditions preventing the amidation of the carboxylic acids. The protonated amino acid components were then heated at reflux at 110° C. in toluene to form the ester compounds. The Dean Stark trap was used to collect the water biproducts, increasing the yield of the reaction.

¹H NMR (300 MHz, DMSO-d₆): δ=8.45 (br, 6H, ⁺NH₃—), 7.00-7.53 (m, 18H, aromatic), 4.30 (t, 2H, ⁺NH₃CHCOO—), 4.00 (t, 4H, —COOCH₂CH₂—), 2.90-3.25 (m, 4H, —CHCH₂—Ar), 2.29 (s, 6H, CH₃Ar—), 1.25-1.50 (br, 4H, —COOCH₂CH₂CH₂—), 0.95-1.15 (br, 4H, —COOCH₂CH₂CH₂—). ¹³C NMR (75 MHz, DMSO-d₆): δ=169.1, 145.0, 138.2, 134.7, 129.4, 128.6, 128.3, 127.3, 125.6, 65.5, 53.4, 36.2, 27.7, 24.7, 20.8.

### Example 6

Synthesis of Di-p-toluenesulfonic Acid Salt of Bis-L-phenylalanine-octane-1,8-diester (1-PHE-8)

1,8-octanediol (10.00 g, 0.068 mol), L-phenylalanine (25.79 g, 0.156 mol), p-toluenesulfonic acid monohydrate (31.07 g, 0.163 mol) and toluene (200 mL) were mixed in a 500 mL round-bottom flask equipped with Dean-Stark trap and a magnetic stir bar. The system was heated to reflux for 20 h. After the reaction mixture was cooled to ambient temperature, the product was filtered and washed with diethyl ether. The solid product was dissolved in 3 L of hot water and decolored using activated carbon black (2.00 g) for 2-3 min. After hot filtration and cooling to room temperature, a white solid product was obtained by vacuum filtration. The product was then recrystallized with water for three times to yield 45.9 g (yield 86%) white powders as product. ¹H NMR (500 MHz, DMSO-d₆): 1.08-1.22 (m, 8H), 1.37-1.49 (m, 4H), 2.29 (s, 6H), 3.02 (dd, J=14.06, 7.95 Hz, 2H), 3.14 (dd, J=13.94, 5.87 Hz, 2H), 3.98-4.08 (m, 4H), 4.28 (dd, J=7.83, 6.11 Hz, 2H), 7.11 (dd, J=8.44, 0.61 Hz, 4H), 7.20-7.36 (m, 10H), 7.48 (d, J=7.83 Hz, 4H), 8.36 (br. s., 6H). ¹³C NMR (125 MHz, DMSO-d₆): 21.25, 25.49, 28.20, 28.86, 36.65, 53.83, 65.96, 125.99, 127.65, 128.65, 128.97, 129.76, 135.18, 138.56, 145.46, 169.47.

### Example 7

Synthesis of Poly(ester urea) Homopolymers and Copolymers.

The synthesis of all polymers was adapted from methods described previously (See, e.g., N. Z. Dreger, Z. Fan, Z. K. Zander, C. Tantisuwanno, M. C. Haines, M. Waggoner, T. Parsell, C. S. Sondergaard, M. Hiles, C. Premanandan, M. L. Becker, Amino acid-based Poly(ester urea) copolymer films for hernia-repair applications, Biomaterials, 182 (2018) 44-57 and J. Yu, F. Lin, P. Lin, Y. Gao, M. L. Becker, Phenylalanine-Based Poly(ester urea): Synthesis, Characterization, and in vitro Degradation, Macromolecules, 47 (2014) 121-129, the disclosures of which are incorporated herein by reference in their entirety), and is outlined in Scheme 5, below. Briefly, an interfacial polymerization of p-toluenesulfonic acid monomer salts was performed by dissolving the monomers with desired molar equivalents (1 eq. total) with sodium carbonate (3.1 eq.) in distilled water (0.25 M Na₂CO₃, 35° C.) in a 5 L three-neck round-bottom flask. The solution was equipped with an overhead mechanical stir rod and allowed to stir until the monomers were dissolved (solution was clear). The reaction was then placed in an ice bath and cooled to 0° C. In a 500 mL round bottom flask, triphosgene (0.4 eq.) was dissolved in chloroform and subsequently added to the reaction vessel slowly. The solution turned white upon addition of the chloroform mixture and was stirred for 24 hours. The product was then transferred to a separatory funnel. The reaction mixture was precipitated into hot water to remove chloroform and starting material impurities. The polymer was collected, frozen in liquid nitrogen, and then dried under reduced pressure to remove residual water. If impurities were present in NMR analysis, additional purification steps were performed by dissolving the polymer in acetone and re-precipitating the solution into water.

A general mechanism for synthesizing a counter-ion protected amino acid-based poly(ester urea) polymer or copolymer according to one or more embodiments of the present invention is shown in Scheme 5, below.

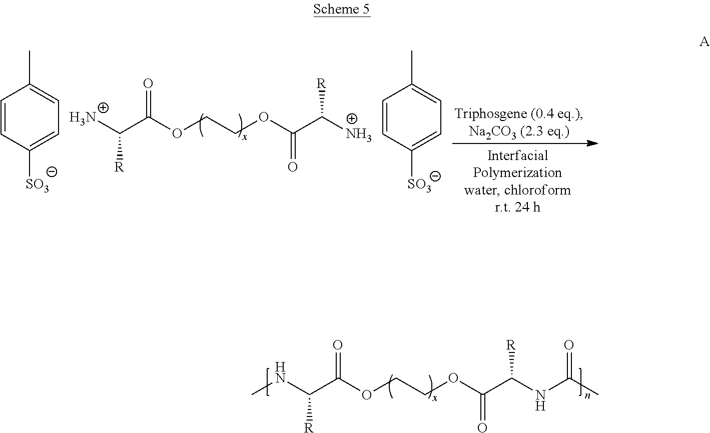

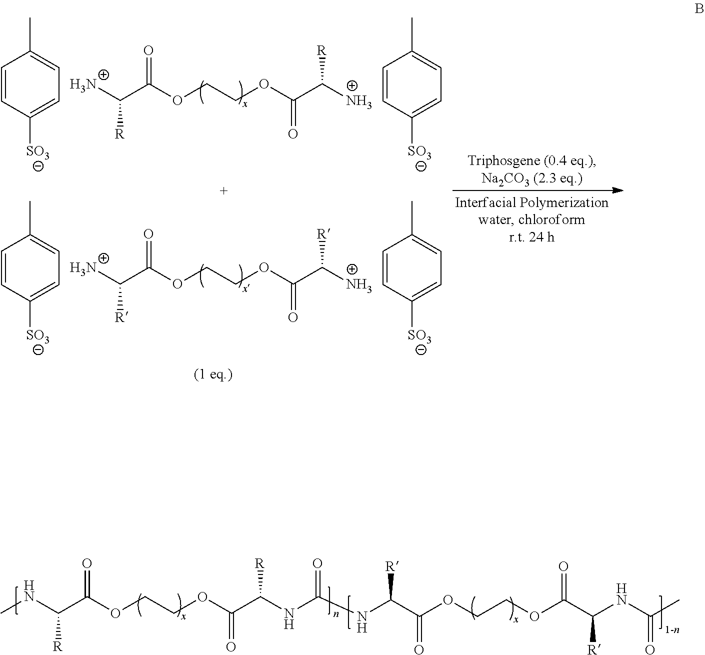

Scheme 5 shows a synthetic route used for the formation of poly(ester urea)s via interfacial polymerization of L-amino acid monomer salts. Homopolymers were synthesized with one composition (type) of monomer salt (1 eq.) dissolved in water and triphosgene (0.4 eq) in chloroform were added. An excess of sodium carbonate (2.3 eq.) was also added to neutralize the triphosgene. PEU copolymers were synthesized in a similar fashion, but with the addition of two compositions (types) of monomer salts (B).

The resulting polymers were analyzed by ¹H NMR as follows: Poly[(1-VAL-10)] (V10) ¹H NMR (300 MHz, 303 K, DMSO-d₆): δ=0.81-0.87 (m, 12H, —CH(CH₃)₂), 1.95-2.01 (m, 2H, —NHCH(CH(CH₃)₂)C(O)O—), 6.37-6.40 (d, J_{H-H}=8.8 Hz, 2H, —NHCH(CH(CH₃)₂)C(O)O—), 1.24, 1.53, 4.01-4.14 (all remaining diol protons). (M_{w}=217 kDa, Mₙ=143 kDa, Ðₘ=1.5, T_{g}=42° C., T_{d}=275° C.) (50-58% yield)

Poly[(1-VAL-8)_{0.70}-co-(1-PHE-6)_{0.30}]. (30P6V8). ¹H NMR (300 MHz, 303 K, DMSO-d₆): δ=0.81-0.87 (m, 12H, —CH(CH₃)₂), 1.93-1.99 (m, 2H, —NHCH(CH(CH₃)₂)C(O)O—), 2.90-2.92 (m, 4H, —NHCH(CH₂Ph)C(O)O—), 4.35-4.40 (m, 2H, —NHCH(CH₂Ph) C(O)O—), 6.36-6.39 (d, J_{H-H}=9.2 Hz, 2H, —NHCH(CH(CH₃)₂)C(O)O—), 6.49-6.52 (d, ³J_{H-H}=8.9 Hz, 2H, —C(O)NHC(CH₂Ph)HC(O)—), 7.16-7.25 (m, 10H, —C₆H₅), 1.18-1.25, 1.45-1.53, 3.97-4.03 (all remaining diol protons) ppm. (M_{w}=104 kDa, Mₙ=52 kDa, εₘ=2.0, T_{g}=45° C., T_{d}=263° C.) (81-90% yield) Poly[(1-VAL-8)₈₀-co-(1-PHE-6)₂₀]. (20P6V8). ¹H NMR (300 MHz, 303 K, DMSO-d₆): δ=0.82-0.85 (m, 12H, —CH(CH₃)₂), 1.95-2.02 (m, 2H, —NHCH(CH(CH₃)₂)C(O)O—), 2.89-2.97 (m, 4H, —NHCH(CH₂Ph)C(O)O—), 4.35-4.42 (m, 2H, —NHCH(CH₂Ph) C(O)O—), 6.37-6.40 (d, J_{H-H}=9.2 Hz, 2H, —NHCH(CH(CH₃)₂)C(O)O—), 6.50-6.53 (d, ³J_{H-H}=9.0 Hz, 2H, —C(O)NHC(CH₂Ph)HC(O)—), 7.15-7.30 (m, 10H, —C₆H₅), 1.19-1.26, 1.46-1.54, 3.97-4.06 (all remaining diol protons) ppm. (M_{w}=88 kDa, Mₙ=56 kDa, D_{M}=1.6, T_{g}=57° C., T_{d}=278° C.) (73-81% yield)

Poly[(1-VAL-8)₉₀-co-(1-PHE-6)₁₀]. (10P6V8). ¹H NMR (300 MHz, 303 K, DMSO-d₆): =0.82-0.87 (m, 12H, —CH(CH₃)₂), 1.97-2.00 (m, 2H, —NHCH(CH(CH₃)₂)C(O)O—), 2.89-2.97 (m, 4H, —NHCH(CH₂Ph)C(O)O—), 4.33-4.39 (m, 2H, —NHCH(CH₂Ph) C(O)O—), 6.37-6.40 (d, J_{H-H}=8.9 Hz, 2H, —NHCH(CH(CH₃)₂)C(O)O—), 6.50-6.53 (d, ³J_{H-H}=9.3 Hz, 2H, —C(O)NHC(CH₂Ph)HC(O)—), 7.15-7.29 (m, 10H, —C₆H₅), 1.18-1.26, 1.45-1.54, 3.96-4.03 (all remaining diol protons) ppm. (M_{w}=107 kDa, Mₙ=68 kDa, D_{M}=1.6, T_{g}=44° C., T_{d}=245° C.) (68-73% yield)

### Example 8

### Blade Coating PEU Films.

Polymer solutions of V₁₀, P6₁₀V8₉₀, P6₂₀V8₈₀, and P6₃₀V8₇₀ were prepared by dissolving 25-45% of polymer in acetone (w/w) to obtain variety in film thicknesses. Drug loadings were 20 or 40% etoricoxib by weight (drug/polymer). The solution was left for 24 hours in an incubator (37° C., 80 rpm) to afford a homogenous mixture. Once thoroughly mixed, solutions were fed into a well where a polyethylene terephthalate (PET) substrate and a Doctor blade (gap height of 0.5-1 mm) were stationed (See, FIG. 8A). Using an EC-100 Fixed Speed Drawdown Coater, the doctor blade was pushed at a constant velocity, drawing the solution cast film out onto the substrate as shown in (FIGS. 8A-C). The films were dried for a minimum of 24 hours under ambient conditions to allow for the film to set with minimal defects and dried further by freeze-drying and lyophilization to ensure that residual solvent had been removed prior to additional testing. (See FIG. 8D) Film thickness was measured using calipers at various locations across the film to ensure a uniform thickness (n=4).

### Example 9

### Drug Content Uniformity.

Samples were taken from different sections of the solution cast films formed in Example 8. The films were placed in a 50:50 mixture of ethanol/THF and sonicated in a water bath (45° C.). Samples were diluted with equal amounts of phosphate buffer to the organic mixture and etoricoxib content was quantified using an Agilent 1290 Infinity high-pressure liquid chromatograph system equipped with a UV-Visible (260 nm) detector (HPLC). Drug content from each sample was then averaged to give the amount of etoricoxib in each film. This value was then compared to a theoretical value (calculated by the weight of the film multiplied by the drug loading) and the accuracy of the theoretical method was considered. Theoretical accuracy was determined by the following equation:

$$Theoretical Accuracy ( \% ) = \frac{Theoretical amount of etoricoxib ( mg )}{Actual amount of etoricoxib ( mg )} \times 100$$

This process was also carried out for the films used for release after they were retired from the study (n=3) to get an accurate measure of the cumulative drug release (actual label claim, %).

### Example 10

### In Vitro Drug Release.

Drug release experiments were conducted on 4 cm² sections of the PEU films. The exact mass and thickness of each sample was measured using a laboratory balance and calipers respectively. The drug-loaded films were placed into magnetic baskets that were then placed into the vessels of an Agilent USP Apparatus 7 equipped with an auto-sampler (Merck & Co., Inc.). The vessels were filled with 10 mL of phosphate-buffered solution (PBS) and samples were taken at programmed time points (1 hr, 2 hr, 4 hr, 6 hr, 12 hr, and 1 to 7 or 1 to 14 days). The agitation rate of the vessels was 40 dips per minute. The entire media volume was replaced after each sample collection. The samples were analyzed using HPLC analysis. Once retired from the study, the remaining etoricoxib in the film was determined by the content uniformity methods described above in Example 9.

### Example 11

In Vivo Release Model of Etoricoxib from PEU Films

Formulations. The formulations were made of valine-based PEU homopolymer (V10) or phenylalanine/valine based PEU copolymers (P6_{N-1}V8_{N}) containing etoricoxib with 20% drug load using the methods described in Examples 1-8, above. The film formulations were fabricated in large sheets and cut to size for desired dose (resultant thickness of 150 μm-250 μm). The films were gamma irradiated for sterilization. Based on previous research, this type of radiation was not expected to cause any changes in polymer structure. Nevertheless, in vitro release on sterilized films was conducted to assure the absence of unusual or erratic release that might suggested chain scission. The target dose for etoricoxib was 9 mg in a 4 cm² film. The general properties of the amino acid-based PEU polymers used in these experiments are set forth in Table 15, below.

TABLE 15
Polymer characterization data

| | M_{w} | Mₙ | | T_{g} | T_{d} |
|---|---|---|---|---|---|
| Polymer | (kDa) | (kDa) | Ðₘ | (° C.) | (° C.) |
| P6₁₀V8₉₀ | 89 | 43 | 2.1 | 43 | 293 |
| P6₂₀V8₈₀ | 132 | 63 | 2.1 | 45 | 305 |
| P63₀V8₇₀ | 94 | 44 | 2.1 | 45 | 288 |
| V10 | 110 | 55 | 2.0 | 42 | 282 |

IV Dosing. Male Sprague-Dawley rats were prepared by Taconic Farms (Germantown, NY) with a dual cannula model having cannula implanted in the jugular vein and carotid artery. The animals (n=3) were fasted overnight with free access to water and were fed six hours after dosing. Etoricoxib was formulated as a solution in DMSO at 4 mg/ml concentration and administered at 2 mg/kg by bolus IV injection into the carotid artery cannula. Systemic blood samples were taken from the jugular vein cannula at 5 min, 15 min, 30 min, 1 hr, 2 hr, 4 hr, 6 hr, 8 hr, 12 hr, 16 hr and 20 hr. Plasma was harvested by centrifugation and stored at −20° C. until analyzed.

PO Dosing. Etoricoxib was dissolved in a small volume (1% of the total volume) of DMSO followed by suspension in 0.5% methyl cellulose (w/v) for administration by oral gavage. Male Sprague-Dawley rats weighing 260 to 310 g were prepared by Taconic Farms (Germantown, NY) with a cannula implanted in the right jugular vein of each animal for blood sampling. The animals (n=4 per dosage) were fasted overnight with free access to water and were fed six hours after dosing. Etoricoxib was administered at 5 mg/kg (5 mL/kg) by oral gavage. The animals were housed in individual cages during the 30-hr sampling period. Systemic plasma samples were taken at 15 min, 30 min, 1 hr, 2 hr, 4 hr, 6 hr, 8 hr, 12 hr, 14 hr, 16 hr, 24 hr and 30 hr. Plasma was harvested by centrifugation and stored at −20° C. until analyzed.

SO Dosing. Male Wister Han rats (300-380 g) were used for the in vivo release studies. Studies were conducted under a protocol approved by the Merck & Co., Inc., West Point IACUC. With the animals under short acting anesthesia, the surgical site (scapular region) was shaved and prepared. A hemostat was inserted into the incision. By opening and closing the jaws of the hemostat, a subcutaneous pocket was created where the film was inserted. Staples were used to seal the incision site. Systemic plasma samples were taken at 15 min, 30 min, 1 hr, 2 hr, 4 hr, 6 hr, 24 hr, 2, 3, 4, 7, 9, and 11 days post implantation. The plasma was separated by centrifugation (10 minutes at 2500 g) and kept frozen at −70° C. until analysis by LC-MS/MS. Upon completion of the study the animals were sacrificed and any remaining film and tissue surrounding the film were collected and frozen until analysis.

Pharmacokinetic Sample Analysis. For reference IV and PO samples, etoricoxib concentrations in rat plasma were determined by LC-MS/MS in the positive ion mode. Plasma samples (150 μL) were diluted to 1 mL with 1 M dibasic potassium phosphate and an internal standard was added. The samples were then extracted with methyl-t-butyl ether. After centrifugation, the aqueous phase was frozen in a dry ice/acetone bath and the organic phase decanted to clean tubes. The solvent was evaporated to dryness under nitrogen at 42° C. Residues were reconstituted in 150 μL of water:acetonitrile (70:30 v/v), mixed, and transferred to 96-well plates. After centrifugation of the plates, 20 μL aliquots were injected onto the LC column.

From the SQ dose, plasma sample analysis was conducted via LC/MS. The LC/MS method had a limit of quantitation (LOQ) of 0.004 μM for etoricoxib Tissues from etoricoxib films 10P6V8 and 30P6V8 were cryo-milled (Spex Sample prep 6870 Freezer/Mill) in two cycles of 4 min precool, 2 min cycle time with 11 cycles per seconds (cps), and 2 min cool time. The resultant powder was extracted with ACN to recover the etoricoxib.

Tissue from etoricoxib V10 films were enzymatically digested with 5 mL digestion buffer per gram of tissue. The digestion buffer was 5 mg/mL collagenase in HEPES buffer. Tubes containing tissue in digestion buffer were vortex mixed for 2 minutes then put at 37° C. overnight. To these tubes, 4 mm milling balls were added and were further mixed with ball mill (Geno Grinder 2000) for 10 min. The samples were further homogenized using a hand homogenizer with disposal tips (30 seconds on medium power). The homogenate was diluted 1:1 with acetonitrile and centrifuged a final time with an aliquot of the supernatant taken for analysis via LC/MS.

Pharmacokinetic analysis. Area under the curve (AUC₀-all), maximum plasma concentration (Cₘₐₓ), and time of Cₘₐₓ (Tₘₐₓ) were calculated using a linear trapezoidal, non-compartmental model of Phoenix 1.3. Deconvolution profiles were also calculated using IVIVC wizard in Phoenix 1.3. Means and percent coefficient of variationCV % were calculated using Excel in Microsoft Office 365 ProPlus. (See, FIGS. 8A-D)

Statistical Analysis. Statistical analysis between sample sets were done using Tukey one-way ANOVA to analyze differences between multiple sets with respect to drug-load, polymer composition, or film thickness on drug release at day 7 and diffusivity constants. Data are reported as means±standard error. A p<0.05 was considered statistically significant. (See, FIGS. 6A-C)

Release Kinetics. To more accurately understand the underlying mechanism of etoricoxib release from the films, the data was fit to the Higuchi model (See, e.g., J. Siepmann, N. A. Peppas, Higuchi equation: Derivation, applications, use and misuse, International Journal of Pharmaceutics, 418 (2011) 6-12, the disclosure of which is incorporated herein by reference in its entirety). This model allows for an approximate determination of diffusivity as well as a prediction for total release, which allows for a quantitative comparison between films. The results are shown in FIGS. 3G-I.

### Example 12

### Diabetic Neuropathic Pain Model and Histological Analysis.

Animals. Adult female CD1 mice (8 weeks old) were purchased from Charles River Laboratories. Mice were housed on a 12-hour light and dark cycle at 22±1° C. and had free access to food and water. Animals were randomly assigned, and two to five mice were housed in each cage. The animal experiment was conducted in accordance with the National Institutes of Health Guide for the Care and Use of Laboratory Animals and approved by the Institutional Animal Care and Use Committees of Duke University.

Diabetic Neuropathic Pain Model. Diabetic neuropathic pain was induced via systemic injection of streptozotocin (STZ). STZ was dissolved in saline solution (50 mg/ml) and administered via intraperitoneal route (75 mg/kg, IP). Neuropathic pain typically developed within 1-3 days after STZ injection. Film implantation was conducted 6 days after STZ injection under isoflurane anesthesia. To inhibit the pain conduction from a mouse hind paw, drug-containing films (40% w/w, ˜0.6 mg etoricoxib) were wrapped around the ipsilateral sciatic nerve. (See, FIG. 15A)

Behavioral analysis of pain. Von Frey testing was conducted to assess mechanical pain as we have shown previously. See, e.g., X. Luo, Y. Huh, S. Bang, Q. He, L. Zhang, M. Matsuda, R. R. Ji, Macrophage Toll-like Receptor 9 Contributes to Chemotherapy-Induced Neuropathic Pain in Male Mice, J Neurosci, 39 (2019) 6848-6864, the disclosure of which is incorporated herein by reference in its entirety. Animals were habituated in boxes on an elevated metal mesh floor for at least 2 days before collecting the baseline. A series of von Frey fibers with logarithmically increasing stiffness (0.02-2.56 gram, Stoelting) was applied to the plantar surface of the hind-paw, and paw withdrawal threshold was calculated using the up-down method. See, e.g., W. J. Dixon, Efficient analysis of experimental observations, Annu Rev Pharmacol Toxicol, 20 (1980) 441-462, the disclosure of which is incorporated herein by reference in its entirety. (See, FIGS. 16B-C).

Statistics for behavioral analysis. All data were expressed as the mean±SEM. Behavioral test data were analyzed by one way or two-way ANOVA, followed by Bonferroni's post-hoc test. P<0.05 was considered as statistically significant.

Histology and Inflammatory Response. Incisions were made on the thigh of the animal to expose the muscle. To produce trauma, three cuts were made to the surface of the thigh muscle. The polymer implants (V10, 40% etoricoxib and P6₃₀V8₇₀, 40% etoricoxib) were placed on top of the muscle under the skin and the wound was sutured to close. (See, FIG. 15A) Three controls were used, blank surgery, blank V10 film, and blank P6₃₀V8₇₀, for comparison of inflammatory response induced from both the implant and the drug. (See, FIGS. 15B-C)

The histopathologic response to the films were determined via histology of tissues harvested at day 14. Upon extraction, tissues were placed in tissue cassettes and fixed in 10% neutral buffered formalin solution. Samples were then processed routinely, embedded in paraffin wax, and sectioned at 5 μm. Following microtomy, sections were stained with haemotoxylin and eosin (H&E) and examined by a board-certified veterinary pathologist with experience in implant pathology. See FIGS. 17A-D.

In light of the foregoing, it should be appreciated that the present invention significantly advances the art by providing a drug-loaded amino acid-based poly(ester urea) film for controlled local release of non-opioid analgesic compounds that is structurally and functionally improved in a number of ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

1. A drug-loaded amino acid-based poly(ester urea) film for controlled local release of non-opioid analgesic compounds comprising:
an amino acid-based poly(ester urea) polymer or copolymer; and
a therapeutically effective amount of a non-opioid analgesic compound.

2. The drug-loaded amino acid-based poly(ester urea) film of claim 1 wherein said amino acid-based poly(ester urea) polymer or copolymer comprises a plurality of diester monomer units, said diester monomer units comprising the residues of two amino acids separated by from 6 to 12 carbon atoms.

3. (canceled)

4. The drug-loaded amino acid-based poly(ester urea) film of claim 1 wherein said amino acid-based poly(ester urea) polymer or copolymer is a polymer having the formula: 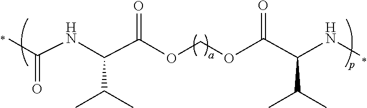
where a is an integer from 6 to 12, and p is an integer from 30 to 300.

5. The drug-loaded amino acid-based poly(ester urea) film of claim 1 wherein said amino acid-based poly(ester urea) polymer or copolymer is a copolymer comprising:
a plurality of first diester monomer units, said first diester monomer units comprising the residues of two amino acids separated by from 6 to 12 carbon atoms; and
a plurality of second diester monomer units, said second diester monomer units comprising the residues of two amino acids separated by from 6 to 12 carbon atoms;
wherein the amino acid residues in said first diester-monomer units are different from the amino acid residues in said second diester monomer units.

6. The drug-loaded amino acid-based poly(ester urea) film of claim 5 wherein said plurality of first diester monomer units comprise the residues of two valine molecules separated by from 6 to 12 carbon atoms.

7. (canceled)

8. (canceled)

9. The drug-loaded amino acid-based poly(ester urea) film of claim 5 wherein said plurality of second diester monomer units comprise the residues of two phenylalanine molecules separated by from 6 to 12 carbon atoms.

10. (canceled)

11. (canceled)

12. The drug-loaded amino acid-based poly(ester urea) film of claim 5 wherein said second diester monomer units comprise from about 10 mol % to about 50 mol % of said amino acid-based poly(ester urea) copolymer.

13. The drug-loaded amino acid-based poly(ester urea) film of claim 5 wherein said amino acid-based poly(ester urea) copolymer is the reaction product of a first ion-protected diester monomer, a second ion-protected diester monomer, and a urea forming compound.

14. (canceled)

15. The drug-loaded amino acid-based poly(ester urea) film of claim 5 wherein said amino acid-based poly(ester urea) copolymer has a formula selected from: 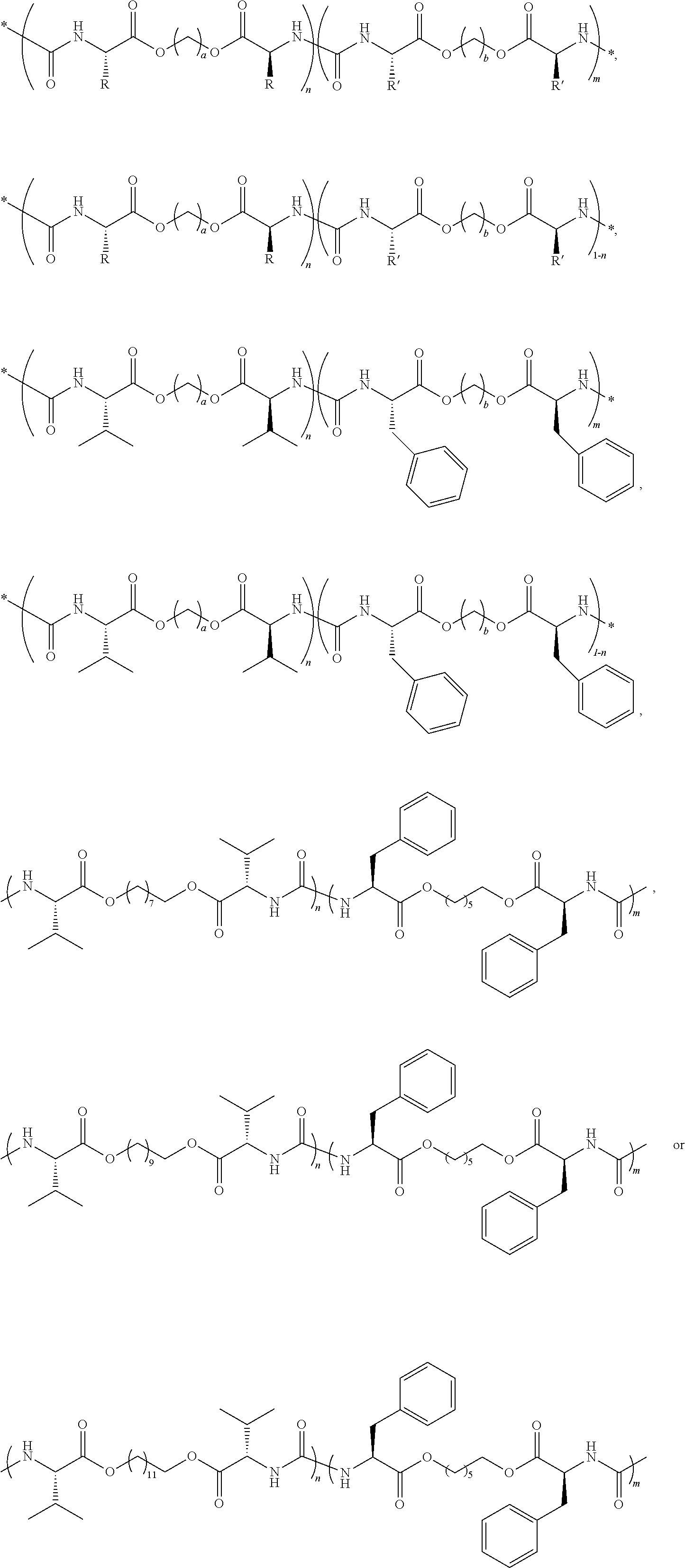
where a is an integer from 5 to 11; b is an integer from 5 to 11; R is —CH(CH₃)₂, —CH₃, —(CH₂)₃NHC(NH₂)C═NH, —CH₂CONH₂, —CH₂COOH, —CH₂SH, —(CH₂)₂COOH, —(CH)₂CONH₂, —NH₂, —CH₂C═CH—N═CH—NH, —CH(CH₃)CH₂CH₃, —CH₂CH(CH₃)₂, —(CH₂)₄NH₂, —(CH₂)₂SCH₃, —CH₂Ph, —CH₂OH, —CH(OH)CH₃, —CH₂—C═CH—NH-Ph, —CH₂ -Ph-OH, or —CH₂C₆H₄OCH₂C₆H₅; R′ is CH₂Ph; n is a mole fraction from 0.9 to 0.6, and in is a mole fraction from 0.1 to 0.4.

16. The drug-loaded amino acid-based poly(ester urea) film of claim 1 wherein said amino acid-based poly(ester urea) polymer or copolymer has a number average molecular weight of from about 10,000 Da to about 80,000 Da.

17. The drug-loaded amino acid-based poly(ester urea) film of claim 1 wherein said non-opioid analgesic compound is selected from the group consisting of etoricoxib, bupivicaine, ropivacaine, lidocaine and pharmaceutically acceptable salts thereof.

18. The drug-loaded amino acid-based poly(ester urea) film of claim 1 wherein said non-opioid analgesic compound is etoricoxib or a pharmaceutically acceptable salt thereof.

19. The drug-loaded amino acid-based poly(ester urea) film of claim 1 wherein the non-opioid analgesic compound is substantially homogeneously distributed throughout the amino acid-based poly(ester urea) polymer or copolymer.

20. The drug-loaded amino acid-based poly(ester urea) film of claim 1 comprising from about 1% to about 50%.

21. The drug-loaded amino acid-based poly(ester urea) film of claim 1 having a thickness of from about 50 μm to about 500 μm

22. The drug-loaded amino acid-based poly(ester urea) film of claim 1 having a thickness of from about 10 m to about 1000 μm.

23. The drug-loaded amino acid-based poly(ester urea) film of claim 1 wherein said film continuously releases the non-opioid analgesic compound for from about 1 to about 187 days.

24. The drug-loaded amino acid-based poly(ester urea) film of claim 1 wherein said film continuously releases the non-opioid analgesic compound for from about 3 to about 10 days.

25. The drug-loaded amino acid-based poly(ester urea) film of claim 1 wherein the non-opioid analgesic compound is released by diffusion.

26. The drug-loaded amino acid-based poly(ester urea) film of claim 18 having a Diffusivity Constant of from about 8.0×10⁻⁷ cm²/s to about 2.0×10⁻⁵ cm²/s.

27. An amino acid-based poly(ester urea) film for controlled local release of at least one of etoricoxib and bupivacaine comprising from about 50 to about 99 weight percent of an amino acid-based poly(ester urea) polymer or copolymer and from about 1 to about 50 weight percent etoricoxib, bupivacaine or a pharmaceutically acceptable salt thereof.

28. The amino acid-based poly(ester urea) film of claim 27, wherein the amino acid-based poly(ester urea) polymer or copolymer has the formula: 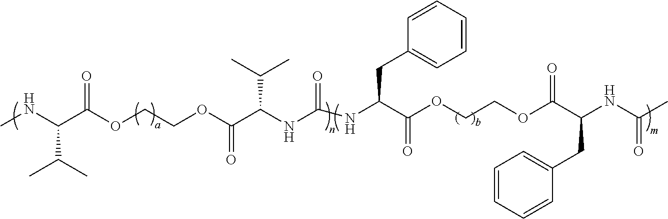
where a is an integer from 5 to 11; b is an integer from 5 to 11; n is a mole fraction from 0.90 to 0.60; and m is a mole fraction from −0.10 to 0.40.

29. The amino acid-based poly(ester urea) film of claim 27, wherein the amino acid-based poly(ester urea) polymer or copolymer has the formula: 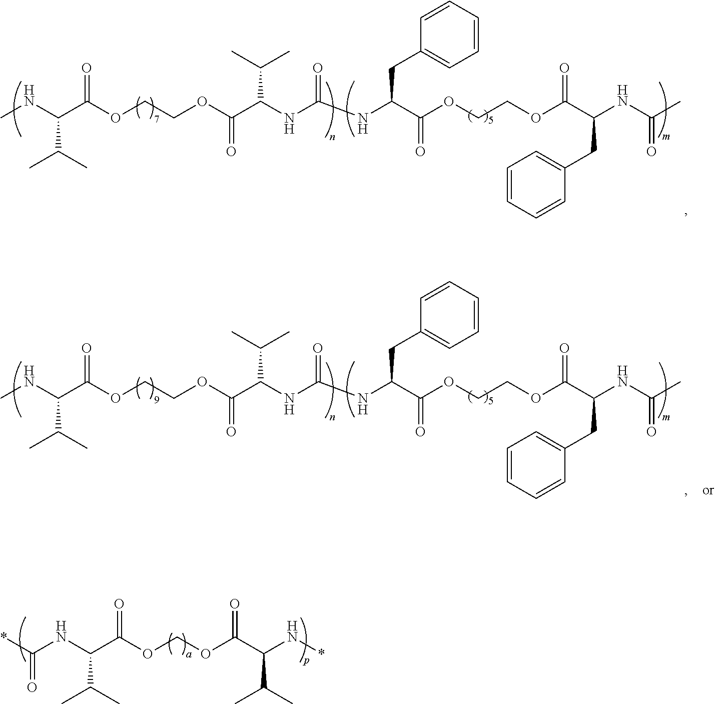
where n is a mole fraction from 0.90 to 0.70; m is a mole fraction from 0.10 to 0.30, and p is an integer from about 30 to about 300.

30. (canceled)

31. The amino acid-based poly(ester urea) film of claim 27,
comprising etoricoxib or a pharmaceutically acceptable salt thereof.

32. The amino acid-based poly(ester urea) film claim 27 comprising bupivacaine or a pharmaceutically acceptable salt thereof.

33. The amino acid-based poly(ester urea) film of claim 27, wherein the amino acid-based poly(ester urea) polymer or copolymer has the formula: 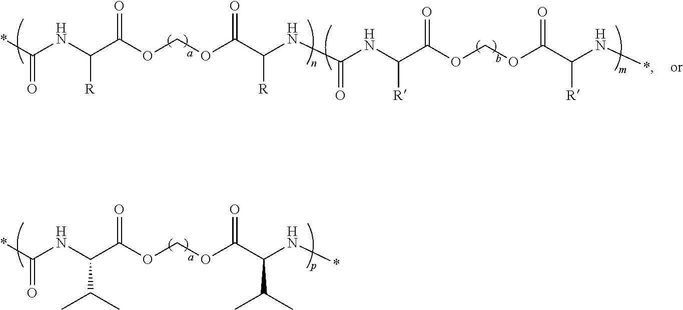
where R is —CH(CH₃)₂, —CH₃, —(CH₂)₃(NH₂)C═NH, —CH₂CON₂, —CH₂COOH, —CH₂SH, —(CH₂)₂COOH, —(CH₂)₂CONH₃, —NH₂, —CH₂C═CH—N═CH—NH, —CH(CH₃)(CH₂CH₃), —CH₂CH(CH₃)₂, —CH₂)₄NH₂, —CH₂)₂SCH₃, —CH₂PH, —CH₂OH, —CH(OH)CH₃, —CH₂—C═CH—NH-Ph, —CH₂-Ph-OH, or —CH₂C₆H₄OCH₂C₆H₅; R′ is CH₂Ph; a is an integer from 6 to 12; b is an integer from 6 to 12; n is a mole fraction from 0.90 to 0.60; and in is a mole fraction from 0.10 to 0.40 and p is an integer from about 30 to about 300.

34. (canceled)

35. The drug-loaded amino acid-based poly(ester urea) film for controlled local release of non-opioid analgesic compounds of claim 5 comprising:
an amino acid-based poly(ester urea) polymer or copolymer having the formula: 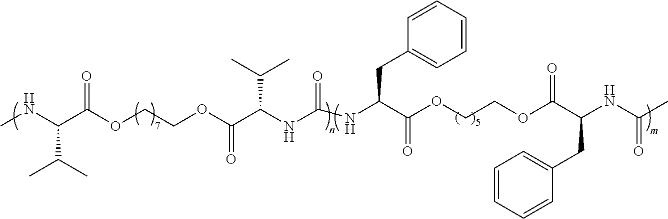
where n is a mole fraction from 0.9 to about 0.7 and m is a mole fraction from about 0.1 to about 0.3; and
from about 20 wt % to about 40 wt % of etoricoxib or pharmaceutically acceptable salts thereof.

36. A method for making the drug-loaded amino acid-based poly(ester urea) film of claim 1 comprising:
A) dispersing or dissolving an amino acid-based poly(ester urea) polymer or copolymer and a non-opioid analgesic compound in a solvent for at least one of said amino acid-based poly(ester urea) polymer or copolymer and said non-opioid analgesic compound;
B) mixing the amino acid-based poly(ester urea) polymer or copolymer/non-opioid analgesic compound dispersion or solution of Step A until the non-opioid analgesic compound is substantially homogeneously distributed throughout the amino acid-based poly(ester urea) polymer or copolymer;
C) solvent casting the substantially homogeneous amino acid-based poly(ester urea) polymer or copolymer and non-opioid analgesic compound composition of Step B onto a substrate to form a non-opioid analgesic compound loaded amino acid-based poly(ester urea) polymer or copolymer film;
D) allowing the non-opioid analgesic compound loaded amino acid-based poly(ester urea) polymer or copolymer film to dry under ambient conditions; and
E) removing any remaining solvent by lyophilization or vacuum drying.

37. The method of claim 36 wherein said amino acid-based poly(ester urea) polymer or copolymer has the formula: 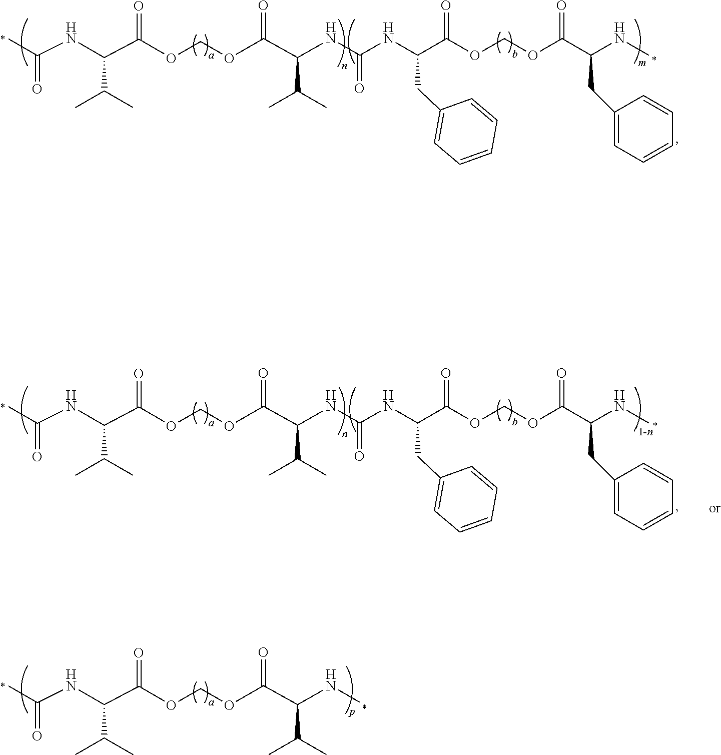
where a is an integer from 6 to 12; b is an integer from 6 to 12; n is a mole fraction from 0.90 to 0.60, m is a mole fraction from 4-0.10 to 0.40, and p is an integer from 30 to 300.

38. (canceled)

39. The method of claim 36 wherein the non-opioid analgesic compound is selected from the group consisting of etoricoxib, bupivacaine, ropivacaine, lidocaine and pharmaceutically acceptable salts thereof.

40. The method of claim 36 wherein said non-opioid analgesic compound is etoricoxib or a pharmaceutically acceptable salt thereof.

41. (canceled)

42. (canceled)

43. The method of claim 36 wherein the amino acid-based poly(ester urea) polymer or copolymer/non-opioid analgesic compound composition of Step B comprises from about 1% to about 50% of said non-opioid analgesic compound by weight.

44. The method of claim 36 wherein the step of mixing (step B) comprises mixing the composition of step B in an incubator at a temperature of from about 25° C. to about 45° C. for at least 24 hours.

45. (canceled)

46. (canceled)

47. (canceled)

48. The method of claim 36 wherein said step of allowing (step D) comprises allowing the non-opioid analgesic compound loaded amino acid-based poly(ester urea) polymer or copolymer film to dr under ambient conditions for at least 24 hours.

49. The method of claim 36 comprising:
A) dispersing or dissolving said amino acid-based poly(ester urea) polymer or copolymer having a formula selected from: 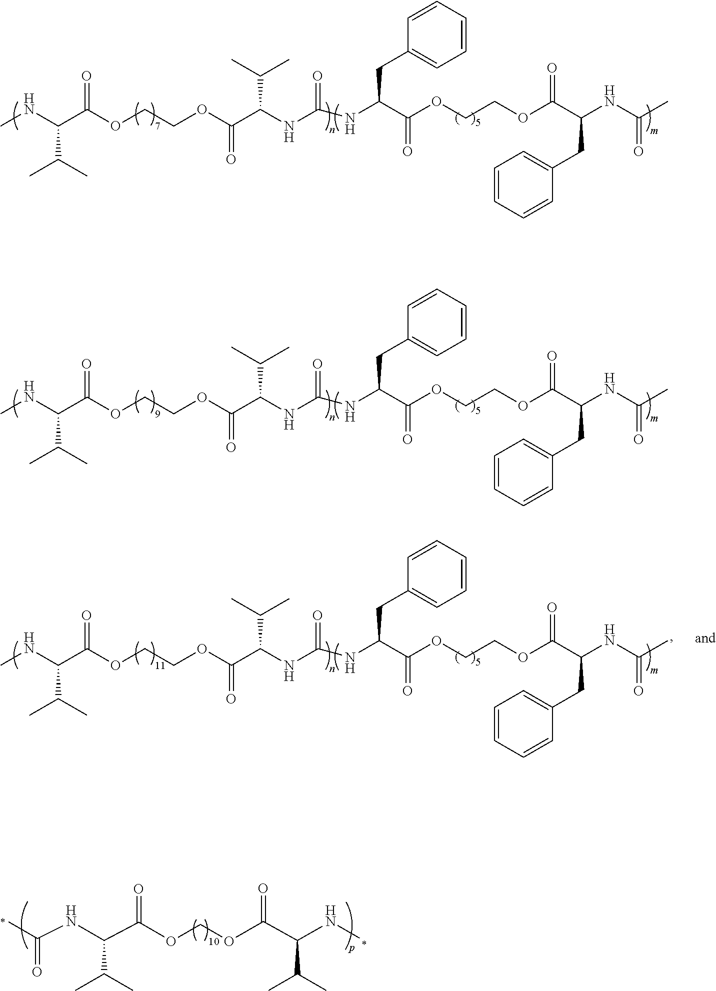
where n is a mole fraction from 0.9 to about 0.7 and in is a mole fraction from about 0.1 to about 0.3, and p is an integer from 30 to 300 and a non-opioid analgesic compound selected from the group consisting of etoricoxib, bupivacaine, ropivacaine, lidocaine and pharmaceutically acceptable salts thereof in a solvent selected from the group consisting of acetone, 1,4-dioxane, methylene chloride, ethyl acetate, dimethyl sulfoxide, chloroform, methyl tetrahydrofuran, tetrahydrofuran and combinations thereof, where said non-opioid analgesic compound is form 20% to 40% of the total weight of said amino acid-based poly(ester urea) polymer or copolymer and said non-opioid analgesic compound;
B) mixing the amino acid-based poly(ester urea) polymer or copolymer/non-opioid analgesic compound dispersion or solution of Step A in an incubator at a temperature of from about 25° C. to about 45° C. for at least 24 hours to afford a substantially homogenous mixture of said non-opioid analgesic compound and said amino acid-based poly(ester urea) polymer or copolymer;
C solvent casting the substantially homogeneous amino acid-based poly(ester urea) polymer or copolymer and non-opioid analgesic compound composition of Step B onto a removable PET substrate using a Doctor blade apparatus;
D) allowing the non-opioid analgesic compound loaded amino acid-based poly(ester urea) polymer or copolymer film to dry under ambient conditions for at least 24 hours to form a non-opioid analgesic compound loaded amino acid-based poly(ester urea) polymer or copolymer film; and
E) removing any remaining solvent in the non-opioid analgesic compound loaded amino acid-based poly(ester urea) polymer or copolymer film by lyophilization or vacuum drying.

50. A method for the localized treatment of a painful condition in a human or other mammal using the drug-loaded amino acid-based poly(ester urea) film of claim 1 comprising:
A) identifying an area of the body of said human or other mammal where said painful condition exists; and
B) preparing and inserting said drug-loaded amino acid-based poly(ester urea) film into the body of said human or other mammal at or in close proximity to said identified area, wherein said drug-loaded amino acid-based poly(ester urea) film is sized to deliver a therapeutically effective amount of a non-opioid analgesic compound for a predetermined time period.

51. (canceled)

52. (canceled)
